(19) United States
(12) Patent Application Publication
(10) Pub. No.: US 2025/0305064 A1
(43) Pub. Date: Oct. 2, 2025

(54) PHASED SEQUENCING INFORMATION FROM CIRCULATING TUMOR DNA

(71) Applicant: Flagship Pioneering Innovations VI, LLC, Cambridge, MA (US)

(72) Inventor: Anthony P. Shuber, Cambridge, MA (US)

(21) Appl. No.: 19/235,350

(22) Filed: Jun. 11, 2025

Related U.S. Application Data
(63) Continuation of application No. PCT/US23/83601, filed on Dec. 12, 2023.
(60) Provisional application No. 63/432,008, filed on Dec. 12, 2022.

(51) Int. Cl. C12Q 1/6886; C12Q 1/6874; G16B 30/10; G16B 40/20

(52) U.S. Cl. CPC C12Q 1/6886; C12Q 1/6874; G16B 30/10; G16B 40/20; C12Q 2600/154; C12Q 2600/156

(57) ABSTRACT

Disclosed herein are methods, non-transitory computer readable media, and systems for determining a signal informative for presence or absence of a cancer in a sample. Generally, the signal includes phased sequencing information of cell-free DNA in which methylation sequence information and/or mutation sequence information can be attributed to various sources (e.g., to a maternal chromosome or to a paternal chromosome). Individual-specific differences between the maternal and paternal chromosomes can be informative markers to create haplotype-specific sequence information (e.g., phase sequencing information) informative for presence or absence of cancer.

### CROSS-REFERENCE TO RELATED APPLICATIONS

This application claims the benefit of and priority to U.S. Provisional Patent Application No. 63/432,008 filed Dec. 12, 2022, the entire disclosure of which is hereby incorporated by reference in its entirety for all purposes.

### BACKGROUND

Conventional detection methods involve analyzing a wealth of information to determine presence of a disease in a patient. However, not all information may be relevant or informative. Including such information in the analysis can have a confounding effect and therefore, are detrimental towards the final predictive accuracy. Thus, there is a need to improve predictive accuracy by identifying more relevant signatures.

### SUMMARY

Disclosed herein are methods, non-transitory computer readable media, and systems for determining a signal informative for presence or absence of a cancer in a sample obtained from an individual. Generally, the signal includes phased sequencing information, also referred to herein as haplotype sequencing information, which represents sequencing information derived specifically from a particular source, examples of which include a maternal chromosome source and/or a paternal chromosome source. In various embodiments, the phased sequencing information includes methylation statuses for a plurality of genomic sites. Thus, the phased sequencing information may include methylation statuses of genomic sites from a common source (e.g., same maternal chromosome or same paternal chromosome). For example, cancer-related methylation at one genomic site may be coupled with methylation at a second genomic site on the same maternal or paternal chromosome. Detecting this coupling between two or more genomic sites provides disease diagnostic utility. Thus, methylation statuses of multiple genomic sites from a common source can be included in the signal informative for presence or absence of a cancer.

Disclosed herein is a method for determining a signal informative for presence or absence of a cancer in a sample obtained from an individual, the method comprising: obtaining or having obtained sequence reads of cell-free DNA from the sample; obtaining or having obtained long sequence reads of reference nucleic acids, wherein the long sequence reads of reference nucleic acids are at least 500 bases in length; attributing long sequence reads of reference nucleic acids to one of two or more different sources of the individual; and generating phased sequencing information of cell-free DNA by aligning the obtained sequence reads of cell-free DNA to the long sequence reads of reference nucleic acids. In various embodiments, the phased sequencing information of cell-free DNA comprises methylation sequence information of the cell-free DNA. In various embodiments, the methylation information of the cell-free DNA comprises methylation statuses for a plurality of genomic sites. In various embodiments, the methylation statuses for a plurality of genomic sites comprise coupled genomic sites representing two or more genomic sites with methylation patterns that originate from a common source. In various embodiments, generating phased sequencing information of cell-free DNA comprises: comparing methylation statuses of two or more genomic sites from a first source to methylation statuses of the two or more genomic sites from a second source. In various embodiments, the plurality of genomic sites comprise a plurality of CpG sites shown in any of Tables 1-4 or portions of the plurality of CpG sites shown in any of Tables 1-4.

In various embodiments, the phased sequencing information of cell-free DNA comprises mutation sequence information of the cell-free DNA. In various embodiments, the mutation sequence information of the cell-free DNA comprises a plurality of mutations present across the plurality of genomic sites. In various embodiments, the plurality of mutations present across the plurality of genomic sites comprise coupled genomic sites representing two or more mutated genomic sites originating from a common source. In various embodiments, the plurality of mutations comprise one or more of a single nucleotide polymorphism (SNP), single nucleotide variant (SNV), insertion, deletion, copy number variation (CNV), duplication, or translocation.

In various embodiments, the two or more different sources of the individual comprise a maternal chromosome source or a paternal chromosome source. In various embodiments, the long sequence reads of reference nucleic acids comprise at least 500 bases, at least 1000 bases, at least 2000 bases, at least 3000 bases, at least 4000 bases, at least 5000 bases, at least 6000 bases, at least 7000 bases, at least 8000 bases, at least 9000, at least 10,000 bases, at least 12,000 bases, at least 15,000 bases, at least 20,000 bases, at least 25,000 bases, at least 30,000 bases, at least 40,000 bases, at least 50,000 bases, at least 60,000 bases, at least 70,000 bases, at least 80,000 bases, at least 90,000 bases, or at least 100,000 bases. In various embodiments, the long sequence reads of reference nucleic acids comprise between 5,000 bases and 100,000 bases. In various embodiments, generating phased sequencing information of cell-free DNA does not include aligning the obtained sequence reads of cell-free DNA to a reference genome.

In various embodiments, the reference nucleic acids comprise genomic DNA from cells of the individual. In various embodiments, the cells of the individual comprise peripheral blood mononuclear cells (PBMCs) or polymorphonuclear cells. In various embodiments, the cell-free DNA is obtained from a blood sample, and wherein the reference nucleic acids are obtained from a tissue sample. In various embodiments, obtaining or having obtained sequence reads of cell-free DNA comprises performing an assay, wherein the assay comprises one or more of: a. sequencing of target nucleic acids via targeted sequencing, whole genome sequencing, or whole genome bisulfite sequencing; b. a nucleic acid amplification assay; and c. an assay that generates methylation information. In various embodiments, the nucleic acid amplification assay is a PCR assay. In various embodiments, the PCR assay comprises a real-time PCR assay, quantitative real-time PCR (qPCR) assay, digital PCR (dPCR) assay, allele-specific PCR assay, or reverse-transcription PCR assay. In various embodiments, obtaining or having obtained sequence reads of cell-free DNA comprises performing a target enrichment assay. In various embodiments, the target enrichment assay comprises hybrid capture. In various embodiments, performing the assay comprises: obtaining bisulfite converted target nucleic acids and/or reference nucleic acids; and selectively amplifying target regions of the bisulfite converted target nucleic acids and/or reference nucleic acids. In various embodiments, obtaining or having obtained long sequence reads of reference nucleic acids comprises performing nanopore sequencing of reference nucleic acids. In various embodiments, methods disclosed herein further comprise: generating the signal informative for presence or absence of a cancer using at least the phased sequencing information of cell-free DNA.

Additionally disclosed herein is a non-transitory computer readable medium comprising instructions that, when executed by a processor, cause the processor to: obtain sequence reads of cell-free DNA from the sample; obtain long sequence reads of reference nucleic acids, wherein the long sequence reads of reference nucleic acids are at least 500 bases in length; attribute long sequence reads of reference nucleic acids to one of two or more different sources of the individual; and generate phased sequencing information of cell-free DNA by aligning the obtained sequence reads of cell-free DNA to the long sequence reads of reference nucleic acids. In various embodiments, the phased sequencing information of cell-free DNA comprises methylation sequence information of the cell-free DNA. In various embodiments, the methylation information of the cell-free DNA comprises methylation statuses for a plurality of genomic sites. In various embodiments, the methylation statuses for a plurality of genomic sites comprise coupled genomic sites representing two or more methylated genomic sites originating from a common source. In various embodiments, generating phased sequencing information of cell-free DNA comprises: comparing methylation statuses of two or more genomic sites from a first source to methylation statuses of the two or more genomic sites from a second source. In various embodiments, the plurality of genomic sites comprise a plurality of CpG sites shown in any of Tables 1-4 or portions of the plurality of CpG sites shown in any of Tables 1-4.

In various embodiments, the phased sequencing information of cell-free DNA comprises mutation sequence information of the cell-free DNA. In various embodiments, the mutation sequence information of the cell-free DNA comprises a plurality of mutations present across the plurality of genomic sites. In various embodiments, the plurality of mutations present across the plurality of genomic sites comprise coupled genomic sites representing two or more mutated genomic sites originating from a common source. In various embodiments, the plurality of mutations comprise one or more of a single nucleotide polymorphism (SNP), single nucleotide variant (SNV), insertion, deletion, copy number variation (CNV), duplication, or translocation. In various embodiments, the two or more different sources of the individual comprise a maternal chromosome source or a paternal chromosome source. In various embodiments, the long sequence reads of reference nucleic acids comprise at least 500 bases, at least 1000 bases, at least 2000 bases, at least 3000 bases, at least 4000 bases, at least 5000 bases, at least 6000 bases, at least 7000 bases, at least 8000 bases, at least 9000, at least 10,000 bases, at least 12,000 bases, at least 15,000 bases, at least 20,000 bases, at least 25,000 bases, or at least 30,000 bases. In various embodiments, the long sequence reads of reference nucleic acids comprise between 5,000 bases and 100,000 bases.

In various embodiments, the instructions that cause the processor to generate phased sequencing information of cell-free DNA does not include instructions that cause the processor to align the obtained sequence reads of cell-free DNA to a reference genome. In various embodiments, the reference nucleic acids comprise genomic DNA from cells of the individual. In various embodiments, the cells of the individual comprise peripheral blood mononuclear cells (PBMCs) or polymorphonuclear cells. In various embodiments, the cell-free DNA is obtained from a blood sample, and wherein the reference nucleic acids are obtained from a tissue sample.

Additionally disclosed herein is a system comprising: a processor; a data storage comprising sequence reads of cell-free DNA from a sample obtained from an individual and long sequence reads of reference nucleic acids, wherein the long sequence reads of reference nucleic acids are at least 500 bases in length; and a non-transitory computer readable medium comprising instructions that, when executed by the processor, cause the processor to: attribute long sequence reads of reference nucleic acids to one of two or more different sources of the individual; and generate phased sequencing information of cell-free DNA by aligning the obtained sequence reads of cell-free DNA to the long sequence reads of reference nucleic acids. In various embodiments, the phased sequencing information of cell-free DNA comprises methylation sequence information of the cell-free DNA. In various embodiments, the methylation information of the cell-free DNA comprises methylation statuses of a plurality of genomic sites. In various embodiments, the methylation statuses for a plurality of genomic sites comprise coupled genomic sites representing two or more methylated genomic sites originating from a common source.

In various embodiments, generating phased sequencing information of cell-free DNA comprises: comparing methylation statuses of two or more genomic sites from a first source to methylation statuses of the two or more genomic sites from a second source. In various embodiments, the plurality of genomic sites comprise a plurality of CpG sites shown in any of Tables 1-4 or portions of the plurality of CpG sites shown in any of Tables 1-4. In various embodiments, the phased sequencing information of cell-free DNA comprises mutation sequence information of the cell-free DNA. In various embodiments, the mutation sequence information of the cell-free DNA comprises a plurality of mutations present across the plurality of genomic sites. In various embodiments, the plurality of mutations present across the plurality of genomic sites comprise coupled genomic sites representing two or more mutated genomic sites originating from a common source. In various embodiments, the plurality of mutations comprise one or more of a single nucleotide polymorphism (SNP), single nucleotide variant (SNV), insertion, deletion, copy number variation (CNV), duplication, or translocation.

In various embodiments, the two or more different sources of the individual comprise a maternal chromosome source or a paternal chromosome source. In various embodiments, the long sequence reads of reference nucleic acids comprise at least 500 bases, at least 1000 bases, at least 2000 bases, at least 3000 bases, at least 4000 bases, at least 5000 bases, at least 6000 bases, at least 7000 bases, at least 8000 bases, at least 9000, at least 10,000 bases, at least 12,000 bases, at least 15,000 bases, at least 20,000 bases, at least 25,000 bases, or at least 30,000 bases. In various embodiments, the long sequence reads of reference nucleic acids comprise between 5,000 bases and 100,000 bases.

In various embodiments, generating phased sequencing information of cell-free DNA does not include aligning the obtained sequence reads of cell-free DNA to a reference genome. In various embodiments, the reference nucleic acids comprise genomic DNA from cells of the individual. In various embodiments, the cells of the individual comprise peripheral blood mononuclear cells (PBMCs) or polymorphonuclear cells. In various embodiments, the cell-free DNA is obtained from a blood sample, and wherein the reference nucleic acids are obtained from a tissue sample.

### BRIEF DESCRIPTION OF THE DRAWINGS

The foregoing and other objects, features and advantages of the invention will become apparent from the following description of preferred embodiments, as illustrated in the accompanying drawings. Like referenced elements identify common features in the corresponding drawings. The drawings are not necessarily to scale, with emphasis instead being placed on illustrating the principles of the present invention, in which:

FIG. 1 shows an example flow diagram for determining a signal informative for presence or absence of a cancer in a sample obtained from an individual, in accordance with an embodiment.

FIG. 2A depicts an example conversion of nucleic acids, in accordance with an embodiment.

FIG. 2B shows the results of nitrite conversion on select nucleotides, in accordance with a second embodiment. Figure adapted from Li et al. (2022) Genome Biology 23:122.

FIG. 3 illustrates an example computer for implementing methods in accordance with FIG. 1.

### DETAILED DESCRIPTION

### Definitions

Terms used in the claims and specification are defined as set forth below unless otherwise specified.

The terms “subject,” “patient,” and “individual” are used interchangeably and encompass a cell, tissue, or organism, human or non-human, male or female.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art. Examples of an aliquot of body fluid include amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour. In particular embodiments, the sample is a liquid biopsy sample, such as a blood sample.

The term “obtaining sequence information” encompasses obtaining information that is determined from at least one sample. Obtaining sequence information encompasses obtaining a sample and processing the sample and/or performing an assay on the sample to experimentally determine the sequence information. The phrase also encompasses receiving the information, e.g., from a third party that has processed the sample and/or performed an assay on the sample to experimentally determine the sequence information.

The phrase “target nucleic acids” refers to nucleic acids of an individual that contain at least signatures that may be informative for determining presence or absence of the cancer. The target nucleic acids may further include baseline biological signatures of the individual that are not informative or less informative. In various embodiments, target nucleic acids may be nucleic acids derived from a diseased cell that is associated with the cancer. For example, target nucleic acids may be cell-free nucleic acids originating from cancer cells (also referred to as circulating tumor DNA). Target nucleic acids can be any of DNA, cDNA, or RNA. In particular embodiments, target nucleic acids include DNA.

The phrase “reference nucleic acids” refers to nucleic acids from genomic DNA of cells of the individual. In various embodiments, the cells include peripheral blood mononuclear cells (PBMCs) or polymorphonuclear cells. Reference nucleic acids can be any of DNA, cDNA, or RNA. In particular embodiments, reference nucleic acids include DNA.

It must be noted that, as used in the specification, the singular forms “a,” “an” and “the” include plural referents unless the context clearly dictates otherwise.

### Exemplary Methods

Disclosed herein are methods for using at least phased sequencing information (e.g., sequencing information derived exclusively from a source, examples of which include either the maternal or paternal chromosomes (i.e., haplotype information)) to generate a signal informative for determining presence or absence of a cancer. The phased sequencing information may include mutation sequence information (e.g., mutations that originate from a common source (e.g., a maternal chromosome or a paternal chromosome)) and/or methylation sequencing information (e.g., methylation statuses of genomic sites that originate from a common source (e.g., a maternal chromosome or a paternal chromosome). Generally, phased sequencing information can reveal additional patterns that can be informative for determining presence or absence of cancer. For example, additional patterns can manifest as coupling between two or more genomic sites from a common source. Coupled genomic sites can refer to two or more genomic sites from a common source in which each genomic site has an alteration (e.g., methylated status or a mutation). Furthermore, genomic sites from a first source may have alterations that differ from genomic sites from a second source. For example, genomic sites from a maternal chromosome may each be methylated, whereas the same genomic sites from a paternal chromosome may not be methylated. These individual-specific differences between the maternal and paternal chromosomes could be used as markers to create haplotype-specific sequence information useful for determining presence or absence of a cancer.

In various embodiments, one or more samples are obtained from an individual. In various embodiments, a sample obtained from the individual is a liquid biopsy sample. In various embodiments, the liquid biopsy sample includes cell-free DNA (cfDNA) fragments. In particular embodiments, the liquid biopsy sample includes one or more cells in the sample, wherein the one or more cells include reference nucleic acids, such as genomic DNA. In various embodiments, two different samples are taken, in which a first sample includes cfDNA fragments and a second sample includes one or more cells that include reference nucleic acids, such as genomic DNA.

In various embodiments, samples may be processed to extract the target nucleic acids and reference nucleic acids. In various embodiments, samples can undergo cellular disruption methods (e.g., to obtain genomic DNA) involving chemical methods or mechanical methods. Example chemical methods include osmotic shock, enzymatic digestion, detergents, or alkali treatment. Example mechanical methods include homogenization, ultrasonication or cavitation, pressure cell, or ball mill. In various embodiments, samples can undergo removal of membrane lipids or proteins or nucleic acid purification. Example chemical methods for removing membrane lipids or proteins and methods for nucleic acid purification include guanidine thiocyanate (GuSCN)-phenol-chloroform extraction, alkaline extraction, cesium chloride gradient centrifugation with ethidium bromide, Chelex® extraction, or cetyltrimethylammonium bromide extraction. Example physical methods for removing membrane lipids or proteins and methods for nucleic acid purification include solid-phase extraction methods using any of silica matrices, glass particles, diatomaceous earth, magnetic beads, anion exchange material, or cellulose matrix. Further details of nucleic acid extraction methods are described in Ali et al, Current Nucleic Acid Extraction Methods and Their Implications to Point-of-Care Diagnostics, Biomed Res. Int. 2017; 2017:9306564, which is hereby incorporated by reference in its entirety.

Methods disclosed herein involve performing an assay to generate sequence information for target nucleic acids and/or sequence information for reference nucleic acids. In various embodiments, performing an assay comprises performing any of: a. sequencing of target nucleic acids via targeted sequencing, whole genome sequencing, or whole genome bisulfite sequencing; b. a nucleic acid amplification assay; and c. an assay that generates methylation information. Generally, sequence information for target nucleic acids may include sequence reads of the target nucleic acids. In particular embodiments, sequence information for target nucleic acids includes sequence reads of cell-free DNA from a sample obtained from an individual. Sequence information for reference nucleic acids may include sequence reads of the reference nucleic acids. In various embodiments, the sequence reads of the reference nucleic acids are long sequence reads (e.g., longer than length of sequence reads of cell-free DNA). In various embodiments, the long sequence reads of reference nucleic acids refer to sequence reads of at least 500 bases, at least 1000 bases, at least 2000 bases, at least 3000 bases, at least 4000 bases, at least 5000 bases, at least 6000 bases, at least 7000 bases, at least 8000 bases, at least 9000, at least 10,000 bases, at least 12,000 bases, at least 15,000 bases, at least 20,000 bases, at least 25,000 bases, at least 30,000 bases, at least 40,000 bases, at least 50,000 bases, at least 60,000 bases, at least 70,000 bases, at least 80,000 bases, at least 90,000 bases, or at least 100,000 bases. In particular embodiments, the long sequence reads of reference nucleic acids refer to sequence reads of between 5,000 and 100,000 bases, between 10,000 and 80,000 bases, between 20,000 and 70,000 bases, between 30,000 and 60,000 bases, or between 40,000 and 50,000 bases.

In various embodiments, sequence information of target nucleic acids and/or sequence information of reference nucleic acids refer to statuses for a plurality of genomic sites. Sequence information of target nucleic acids refers to epigenetic statuses (e.g., methylation statuses) across a plurality of genomic sites in the target nucleic acids. In particular embodiments, sequence information of the target nucleic acids includes 2 or more, 3 or more, 4 or more, 5 or more, 6 or more, 7 or more, 8 or more, 9 or more, 10 or more, 15 or more, 20 or more, 25 or more, 30 or more, 40 or more, 50 or more, 100 or more, 200 or more, 300 or more, 400 or more, 500 or more, 750 or more, 1000 or more, 2000 or more, 3000 or more, 4000 or more, 5000 or more, 6000 or more, 7000 or more, 8000 or more, 9000 or more, 10000 or more, 11000 or more, 12000 or more, 13000 or more, 14000 or more, 15000 or more, 16000 or more, 17000 or more, 18000 or more, 19000 or more, or 20000 or more genomic sites. In various embodiments, the plurality of genomic sites are previously identified and selected. For example, the plurality of genomic sites may be one or more CpG sites whose differential methylation are informative for determining whether an individual has a cancer. A CpG site is portion of a genome that has cytosine and guanine separated by only one phosphate group and is often denoted as “5′-C phosphate-G-3′”, or “CpG” for short. Regions with a high frequency of CpG sites are commonly referred to as “CG islands” or “CGIs”. It has been found that certain CGIs and certain features of certain CGIs in tumor cells tend to be different from the same CGIs or features of the CGIs in healthy cells. Herein, such CGIs and features of the genome are referred to herein as “cancer informative CGIs.” Cancer informative CGI can be a “CGI identifier” or reference number to allow referencing CGIs during data processing by their respective unique CGI identifiers. Example CGIs include, but are not limited to, the CGIs shown in the accompanying tables (any of Tables 1-4) which lists, for each CGI, its respective location in the human genome. Additional example CGIs are disclosed in WO2018209361, Table 1 of U.S. Patent Publication 2020/0109456A1, and Tables 2 and 3 of WO2022/133315, which are hereby incorporated by reference in its entirety.

In various embodiments, performing an assay to generate sequence information for a plurality of genomic sites includes the steps of processing nucleic acids of a sample, enriching the processed nucleic acids for pre-selected genomic sequences (e.g., pre-selected informative CGIs), amplifying the genomic sequences to generate amplicons, and quantifying the amplicons including the genomic sequences (e.g., via sequencing such as next generation sequencing or via quantitative methods such as an ELISA, quantitative PCR, allele-specific PCR, or DNA or RNA-based assay). In various embodiments, performing an assay to generate sequence information for a plurality of genomic sites involves a subset of the previously mentioned steps. For example, enriching the processed nucleic acids can be omitted. Therefore, performing an assay may include processing nucleic acids of a sample, amplifying the pre-selected genomic sequences, and quantifying the amplicons including the genomic sequences.

In various embodiments, performing an assay involves processing target nucleic acids and/or reference nucleic acids. In various embodiments, processing nucleic acids includes treating the nucleic acids to capture methylation modifications, e.g., using bisulfite conversion. Bisulfite conversion enables highly efficient conversion of unmethylated cytosines to uracils of DNA from samples such as whole blood or plasma, cultured cells, tissue samples, genomic DNA, and formalin-fixed, paraffin-embedded (FFPE) tissues. Bisulfite conversion can be performed using commercially available technologies, such as Zymo Gold available from Zymo Research (Irvine, CA) or EpiTect Fast available from Qiagen (Germantown, MD). Other techniques include but are not limited to enzymatic methods.

In various embodiments, performing the assay includes enriching for specific sequences in the target nucleic acids and/or reference nucleic acids. In various embodiments, the specific sequences refer to sequences of pre-selected CGIs. In various embodiments, enrichment of pre-selected CGIs can be accomplished via hybrid capture. Examples of such hybrid capture probe sets include the KAPA HyperPrep Kit and SeqCAP Epi Enrichment System from Roche Diagnostics (Pleasanton, CA). For example, hybrid capture probe sets can be designed to hybridize with particular sequences of the target nucleic acids and/or reference nucleic acids, thereby capturing and enriching the particular sequences.

In various embodiments, performing the assay includes performing nucleic acid amplification to amplify the particular sequences of the target nucleic acids and/or reference nucleic acids. Examples of such assays include, but are not limited to performing PCR assays, Real-time PCR assays, Quantitative real-time PCR (qPCR) assays, digital PCR (dPCR), Allele-specific PCR assays, Reverse-transcription PCR assays and reporter assays. For example, given the processed nucleic acids (e.g., bisulfite converted nucleic acids) that are enriched for pre-selected sequences, a PCR assay is performed to amplify the pre-selected sequences to generate amplicons. Here, PCR primers are added to initiate the amplification. In various embodiments, the PCR primers are whole genome primers that enable whole genome amplification. In various embodiments, the PCR primers are gene-specific primers that result in amplification of sequences of specific genes. In various embodiments, the PCR primers are allele-specific primers. For example, allele specific primers can target a genomic sequence corresponding to a pre-selected CGI, such that performing nucleic acid amplification results in amplification of the sequence of the pre-selected CGI.

In various embodiments, performing the assay includes quantifying the nucleic acids including the pre-selected sequences (e.g., informative CGIs). In some embodiments, quantifying the nucleic acids to generate sequence information comprises performing any of real-time PCR assay, quantitative real-time PCR (qPCR) assay, digital PCR (dPCR) assay, allele-specific PCR assay, or reverse-transcription PCR assay. Therefore, the number of methylated, hypermethylated, unmethylated, or partially methylated pre-selected sequences are quantified.

In various embodiments, performing the assay comprises sequencing the target nucleic acids and/or reference nucleic acids. In various embodiments, sequencing comprises performing next generation sequencing methods to generate sequence reads from the target nucleic acids and/or reference nucleic acids. As described herein, sequence reads from reference nucleic acids may be long sequence reads (e.g., greater than 500 bases in length). Generally, long sequence reads include an average read length that is longer than sequence reads obtained through standard sequencing methods. In various embodiments, the long sequence reads of reference nucleic acids refer to sequence reads of at least 500 bases, at least 1 kilobase, at least 2 kilobases (kb), at least 3 kb, at least 4 kb, at least 5 kb, at least 6 kb, at least 7 kb, at least 8 kb, at least 9 kb, at least 10 kb, at least 12 kb, at least 15 kb, at least 20 kb, at least 25 kb, at least 30 kb, at least 40 kb, at least 50 kb, at least 60 kb, at least 70 kb, at least 80 kb, at least 90 kb, at least 100 kb, at least 200 kb, at least 300 kb, at least 400 kb, at least 500 kb, at least 600 kb, at least 700 kb, at least 800 kb, at least 900 kb, at least 1000 kb, at least 1500 kb, or at least 2000 kb. In particular embodiments, the long sequence reads of reference nucleic acids refer to sequence reads of between 5 kb and 100 kb, between 10 kb and 80 kb, between 20 kb and 70 kb, between 30 kb and 60 kb, or between 40 kb and 50 kb. In particular embodiments, long sequence reads of reference nucleic acids refer to sequence reads of greater than about 8 kb, greater than about 9 kb or greater than about 10 kb. In particular embodiments, long sequence reads of reference nucleic acids refer to sequence reads between about 10 kb and about 100 kb, or between about 10 kb and about 2 MB. In various embodiments, generating long sequence reads of reference nucleic acids involves performing nanopore sequencing. Methods for long-read sequencing are known in the art and such methods can be performed using, for example, an Oxford Nanopore instrument (e.g., PromethION™) or Pacific Biosciences Single-Molecule Real-Time (SMRT) sequencing technology.

In various embodiments, performing the assay includes generating phased sequencing information for target nucleic acids and/or reference nucleic acids. As used herein, “phased sequencing information,” also referred to herein as “haplotype sequencing information,” refers to sequencing information derived specifically from a particular source. For example, phased sequencing information or haplotype sequencing information can refer to sequencing information derived from either the maternal or paternal chromosome. Generally, phased sequencing information of target nucleic acids may be useful for determining presence or absence of a cancer because signals originating from the same source (e.g., maternal or paternal chromosome) may provide additional information in comparison to other approaches that merely analyze signals irrespective of the source.

In various embodiments, the phased sequencing information comprises mutation sequence information of the cell-free DNA. For example, mutation sequence information can include one or more mutations present across a plurality of genomic sites. In particular embodiments, the mutation sequence information includes one or more mutations that originate from a common source (e.g., a maternal chromosome or a paternal chromosome). Here, two or more genomic sites derived from a common source that have a particular pattern of mutations (e.g., each having a mutation, some pattern of mutated/non-mutated, or all non-mutated) can be referred to as coupled genomic sites. In various embodiments, a mutation can be any of a single nucleotide polymorphism (SNP), single nucleotide variant (SNV), insertion, deletion, copy number variation (CNV), duplication, or translocation.

In various embodiments, the phased sequencing information comprises methylation sequence information of the cell-free DNA. Methylation sequence information can include methylation statuses across a plurality of genomic sites. In particular embodiments, the methylation sequence information includes methylation statuses of genomic sites from a common source (e.g., a maternal chromosome or a paternal chromosome). As a specific example, methylation at a first genomic site may be coupled with methylation at a second genomic site on the same maternal or paternal chromosome. Two or more genomic sites with a particular methylation pattern (e.g., all methylated, partially methylated, or non-methylated) that originate from the same maternal or paternal chromosome is referred to herein as coupled methylation sites. Example coupled methylation sites may be two or more CGIs disclosed herein (e.g., two or more CGIs disclosed in any of Tables 1-4 or portions of CGIs disclosed in any of Tables 1-4). In various embodiments, two or more genomic sites of coupled methylation sites may be separated by tens, hundreds, or even thousands of bases. Thus, coupled methylation sites include two or more genomic sites from a common source and need not be limited to genomic sites that are close in proximity (e.g., adjacent CpG sites). In various embodiments, coupled methylation sites include 3 or more, 4 or more, 5 or more, 6 or more, 7 or more, 8 or more, 9 or more, 10 or more, 15 or more, 20 or more, 25 or more, 30 or more, 35 or more, 40 or more, 45 or more, 50 or more, 60 or more, 70 or more, 80 or more, 90 or more, 100 or more, 200 or more, 300 or more, 400 or more, 500 or more, 600 or more, 700 or more, 800 or more, 900 or more, or 1000 or more methylation sites from a common source. Thus, detecting these coupled methylation sites may provide disease diagnostic utility.

In various embodiments, generating phased sequencing information for target nucleic acids comprises aligning sequence reads of target nucleic acids to long sequence reads of reference nucleic acids derived from different sources (e.g., either the maternal or paternal chromosome). Long sequence reads of reference nucleic acids originating from different sources can be distinguished due to sequence differences present in the long sequence reads. For example, given a particular chromosome, long sequence reads derived from a maternal chromosome would have sequence differences in comparison to long sequence reads derived from a paternal chromosome. Here, sequence differences can refer to mutations that are present in long sequence reads from one source, but not present in long sequence reads from the second source, and vice versa. Thus, the presence or absence of certain mutations can be useful for distinguishing whether a long sequence read originated from a first source or a second source. Altogether, by comparing sequences of long sequence reads, a first set of long sequence reads with a set of common sequences can be attributed to a first source (e.g., a maternal chromosome) whereas a second set of long sequence reads with a different set of common sequences can be attributed to a second source (e.g., a paternal chromosome). In various embodiments, the different sets of long sequence reads need not specifically be attributed to a maternal chromosome and a paternal chromosome; rather, it is sufficient to distinguish different sets of long sequence reads from a first source and a second source. These long sequence reads from a first source or a second source have sufficiently different sequences to enable phasing of the target nucleic acids (e.g., to determine sources from which target nucleic acids were derived from).

By aligning sequence reads of target nucleic acids to long sequence reads of reference nucleic acids, the long sequence reads of reference nucleic acids serve as digital guides to phase e.g., determine the source of target nucleic acids. For example, target nucleic acids from a first common source (e.g., from a maternal chromosome) can be categorized together based on sequence similarities between the target nucleic acids and the long sequence reads of reference nucleic acids from the first source. Additionally, target nucleic acids from a second common source (e.g., from a paternal chromosome) can be categorized together based on sequence similarities between the target nucleic acids and the long sequence reads of reference nucleic acids from the second source. In contrast to using the standard human genome to align sequence reads of target nucleic acids, using long reads of reference nucleic acids would enable alignment of reference nucleic acids to sequences of the maternal or paternal chromosome Individual-specific differences between target nucleic acids deriving from the maternal and paternal chromosomes could be used as markers to create haplotype-specific sequence information that is informative for determining presence or absence of a cancer.

In various embodiments, phased sequencing information includes phased methylation sequencing information of cfDNA, where at least a first set of the phased methylation sequencing information of cfDNA originates from a first source and at least a second set of the phased methylation sequencing information of cfDNA originates from a second source. In various embodiments, methods for generating phased sequencing information can further include comparing the first set of the phased methylation sequencing information of cfDNA from the first source to the second set of the phased methylation sequencing information of cfDNA from the second source. In particular embodiments, generating phased sequencing information further includes comparing methylation statuses of two or more genomic sites from a first source to methylation statuses of the same two or more genomic sites from a second source. Differences in methylation statuses of genomic sites from the first source and the second source can be valuable for inclusion in the signal informative for determining presence or absence of a cancer. For example if multiple genomic sites from a first source are methylated but the same genomic sites from a second source are unmethylated, this may be an informative signal for presence or absence of a cancer.

In various embodiments, the phased sequencing information can be used to generate the signal informative for determining the presence or absence of cancer. In various embodiments, the signal is the phased sequencing information. In various embodiments, the signal includes information in addition to the phased sequencing information. For example, the signal can include non-phased sequencing information, such as methylation statuses or mutations across a plurality of genomic locations.

In various embodiments, a machine learning model is deployed to analyze the signal informative for determining the presence or absence of cancer. In various embodiments, the signal includes the phased sequencing information which includes coupled genomic sites or coupled CGIs from a first source and/or a second source. Therefore, trained machine learning models analyze the signal, including phased sequencing information, to output a cancer prediction as to whether the individual has cancer. In particular embodiments, the machine learning model analyzes the signal, which includes differences between epigenetic statuses (e.g., methylation statuses) of phased sequencing information of different sources (e.g., methylation statuses of genomic sites derived from different sources, such as a maternal or paternal chromosome) of target nucleic acids. Therefore, trained machine learning models analyze the signal across the genomic sites in the phased sequencing information to output a cancer prediction as to whether the individual has cancer.

### Example Flow Diagram for Determining a Signal Informative for Presence or Absence of a Cancer

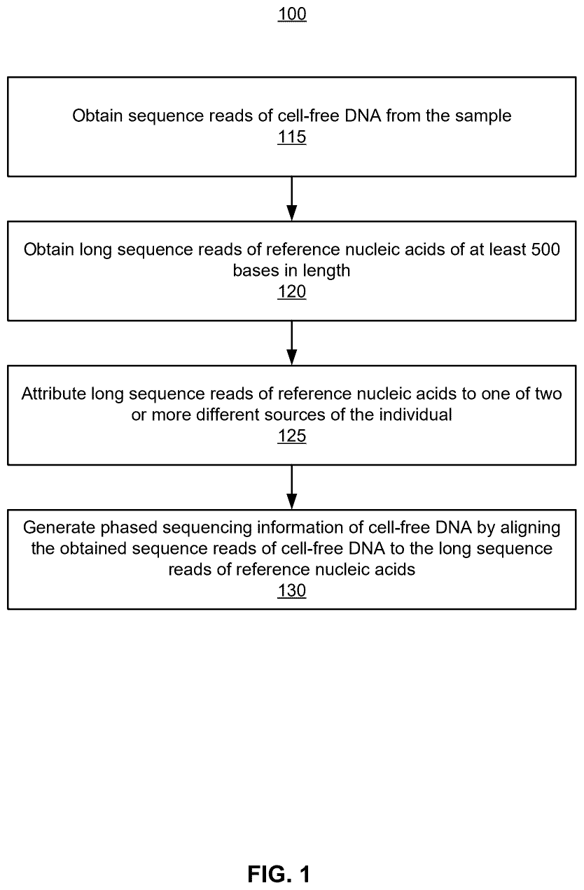

Reference is now made to FIG. 1, which depicts an example flow diagram for determining a signal informative for presence or absence of a cancer in a sample obtained from an individual, in accordance with an embodiment.

Step 115 involves obtaining or having obtained sequence reads of cell-free DNA from a sample.

Step 120 involves obtaining or having obtained long sequence reads of reference nucleic acids, wherein the long sequence reads of reference nucleic acids are at least 500 bases in length.

Step 125 involves attributing long sequence reads of reference nucleic acids to one of two or more different sources of the individual. In various embodiments, the two or more different sources refer to at least a maternal chromosome source and a paternal chromosome source.

Step 130 involves generating phased sequencing information of cell-free DNA by aligning the obtained sequence reads of cell-free DNA to the long sequence reads of reference nucleic acids.

### Longitudinal Monitoring

In various embodiments, methods disclosed herein involve longitudinal monitoring of individual subjects. Performing longitudinal monitoring for individual subjects can be useful for e.g., guiding therapeutic selection and/or administration. In various embodiments, longitudinal monitoring of a subject can include performing the methods described herein, including the methods shown in FIG. 1, two or more times across two or more timepoints.

In various embodiments, performing longitudinal monitoring comprises obtaining samples from a subject and generating predictions (e.g., cancer predictions, such as presence/absence of cancer) across at least two timepoints. In various embodiments, performing longitudinal monitoring comprises obtaining samples from a subject and generating predictions across at least three timepoints. In various embodiments, performing longitudinal monitoring comprises obtaining samples from a subject and generating predictions across at least four timepoints. In various embodiments, performing longitudinal monitoring comprises obtaining samples from a subject and generating predictions across at least five timepoints, at least six timepoints, at least seven timepoints, at least eight timepoints, at least nine timepoints, at least ten timepoints, at least eleven timepoints, at least twelve timepoints, at least thirteen timepoints, at least fourteen timepoints, at least fifteen timepoints, at least sixteen timepoints, at least seventeen timepoints, at least eighteen timepoints, at least nineteen timepoints, or at least twenty timepoints. In various embodiments, the time between any two timepoints can be between 1 day and 12 months, between 5 days and 8 months, between 10 days and 6 months, between 15 days and 4 months, between 20 days and 3 months, between 30 days and 2 months. In various embodiments, the time between any two timepoints can be between 1 days and 10 days, between 10 days and 20 days, between 20 days and 30 days, between 30 days and 40 days, between 40 days and 50 days, or between 50 days and 60 days. In various embodiments, the time between any two timepoints can be between 1 day and 100 days, between 5 day and 80 days, between 10 days and 70 days, between 15 days and 60 days, between 20 days and 50 days, between 25 days and 40 days, or between 30 days and 35 days. In various embodiments, the time between any two timepoints can be between 1 days and 10 days, between 10 days and 20 days, between 20 days and 30 days, between 30 days and 40 days, between 40 days and 50 days, or between 50 days and 60 days. In various embodiments, the time between any two timepoints can be between 1 month and 2 months.

In particular embodiments, methods for longitudinal monitoring involve obtaining a sample from the subject at a first timepoint (e.g., an initial timepoint) and generating a cancer prediction for the sample obtained at the first timepoint. In various embodiments, the first timepoint may refer to a timepoint prior to which the subject receives a therapeutic, such as a cancer therapeutic. Thus, the predicted cancer score for from the sample obtained at the first timepoint may represent a baseline cancer score prior to any therapeutic treatment. In various embodiments, the first timepoint may refer to a timepoint immediately after the subject receives a therapeutic, such as a cancer therapeutic. In this context, “immediately after” the subject receives a therapeutic can refer to a timeframe within 1 day after the subject receives the therapeutic. In various embodiments, “immediately after” refers to a timeframe within 12 hours, within 8 hours, within 6 hours, within 4 hours, within 3 hours, within 2 hours, within 1 hour, within 30 minutes, within 15 minutes, within 10 minutes, within 5 minutes, or within 1 minute of the subject receiving the therapeutic.

In particular embodiments, methods for longitudinal monitoring further involve obtaining one or more subsequent samples from the subject after the first timepoint (e.g., at a second timepoint, at a third timepoint, at a fourth timepoint, etc.) and generating cancer predictions for the one or more subsequent samples. As an example, the cancer predictions from the one or more subsequent samples can be indicative of the progression of the tumor within the subject after the first timepoint. In various embodiments, the one or more subsequent samples are obtained from the subject after the subject has received a therapeutic, such as a cancer therapeutic. Thus, the cancer prediction of the one or more subsequent samples can be reflective of the progression of the tumor within the subject in response to the provided therapeutic.

In various embodiments, longitudinal monitoring is useful for predicting a prognosis for a subject. In various embodiments, based on the longitudinal monitoring of a subject, the subject can be classified in group associated with a particular outcome. For example, the subject can be classified in one of likely to survive or unlikely to survive. As another example, the subject can be classified in one of a responder to a therapeutic or a non-responder to a therapeutic. As another example, the subject can be classified in one of a full responder to a therapeutic, partial responder to a therapeutic, or non-responder to a therapeutic. In various embodiments, the subject can be classified in one of a favorable outcome (examples of which include likely to survive or responder to a therapeutic) or unfavorable outcome (examples of which include unlikely to survive or non-responder to a therapeutic). Thus, a therapeutic can be selected and/or administered to subjects that are classified as a responder to the therapeutic. Additionally, the therapeutic can be withheld from subjects that are classified as a non-responder to the therapeutic.

### Example Nucleic Acids and Methods for Converting Nucleic Acids

In various embodiments, methods disclosed herein involve obtaining or having obtained nucleic acids, such as converted nucleic acids. In various embodiments, converting nucleic acids includes treating the nucleic acids to capture methylation modifications. In various embodiments, converting nucleic acids involves converting one or more unmethylated nucleotides (e.g., cytosines) to another nucleotide (a “converted nucleotide”, as used herein), e.g., using chemical or enzymatic means. In certain embodiments, one or more unmethylated cytosines are converted to a nucleotide that pairs with adenine (e.g., the unmethylated cytosine may be converted to uracil). In certain embodiments, one or more unmethylated adenines are converted to a base that pairs with cytosine (e.g., the unmethylated adenine may be converted to inosine (I)). In certain embodiments, one or more methylated cytosines (e.g., a 5-methylcytosine (5 mC)) is converted to a thymine, which pairs with adenine. In certain embodiments, methylated cytosines are protected from conversion (e.g., deamination) during the conversion step.

After a nucleic acid has been treated to convert unmethylated, or, in some cases, methylated nucleotides, into another nucleotide, the nucleic acid may be amplified. During amplification, the converted nucleotide pairs with its complementary nucleotide, and in the next round of amplification, the complementary nucleotide pairs with a replacement nucleotide. For example, following the conversion of an unmethylated cytosine to a uracil, the nucleic acid may be amplified such that an adenine pairs with the uracil in the first round of replication, and in the second round of replication, the adenine pairs with a thymine. Accordingly, the thymine replaces the uracil in the original nucleic acid sequence, and is referred to herein as a “replacement nucleotide”.

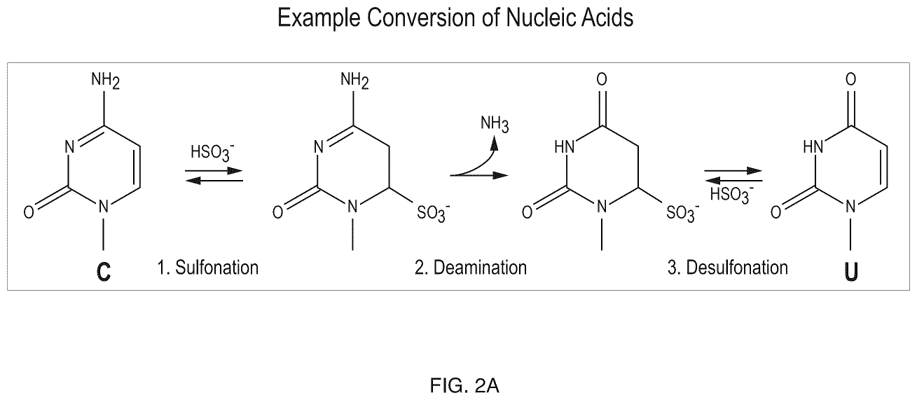

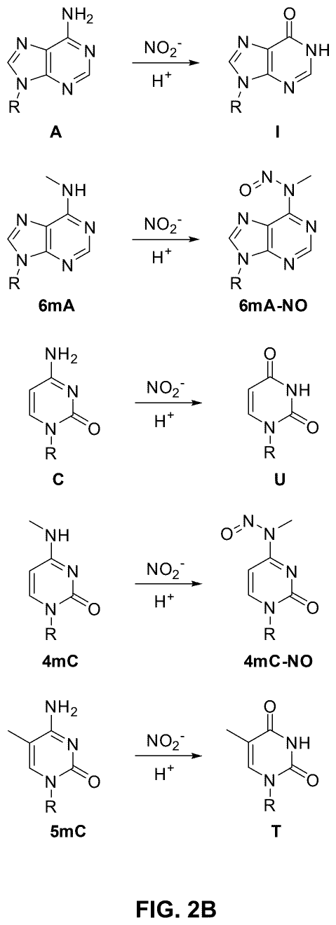

In certain aspects, conversion of the nucleic acids involves selectively deaminating nucleotides. FIG. 2A depicts an example conversion of nucleic acids, in accordance with an embodiment. Selective deamination refers to a process in which unmethylated cytosine residues are selectively deaminated over methylated cytosine (5-methylcytosine) residues. In certain embodiments, deamination of cytosine forms uracil, effectively inducing a C to T point mutation to allow for detection of methylated cytosines. Methods of deaminating cytosine are known in the art, and include chemical conversion (e.g., bisulfite conversion) and enzymatic conversion. In certain embodiments, the enzymatic conversion comprises subjecting the nucleic acid to TET2, which oxidizes methylated cytosines, thereby protecting them, and subsequent exposure to APOBEC, which converts unprotected (i.e., unmethylated) cytosines to uracils.

In some embodiments, the conversion, for example, bisulfite conversion or enzymatic conversion, uses commercially available kits. Bisulfite conversion can be performed using commercially available technologies, such as EZ DNA Methylation-Gold, EZ DNAMethylation-Direct or an EZ DNAMethylation-Lighting kit (Zymo Research Corp (Irvine, California)) or EpiTect Fast available from Qiagen (Germantown, MD). In another example a kit such as APOBECSeq (NEBiolabs) or OneStep qMethyl-PCR Kit (Zymo Research Corp (Irvine, California)) is used.

### Source of Nucleic Acids

Nucleic acids used in the methods described herein can be derived from any source, such as a sample taken from the environment or from a subject (e.g., a human subject). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis. A biological sample can take any of a variety of forms, such as a liquid biopsy (e.g., blood, urine, stool, saliva, or mucous), or a tissue biopsy, or other solid biopsy. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. A biological sample can include any tissue or material derived from a living or dead subject. A biological sample can be a cell-free sample. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc.

The nucleic acid can be of any composition form, such as deoxyribonucleic acid (DNA, e.g., complementary DNA (cDNA), genomic DNA (gDNA) and the like), and/or DNA analogs (e.g., containing base analogs, sugar analogs and/or a non-native backbone and the like), and/or ribonucleic acid (RNA) and or RNA analogs, all of which can be in single- or double-stranded form. In certain embodiments, single-stranded nucleic acids can be made double stranded prior to cutting with an enzyme. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures. Nucleic acids can comprise protein (e.g., histones, DNA binding proteins, and the like). Nucleic acids analyzed by processes described herein can be substantially isolated and are not substantially associated with protein or other molecules. Nucleic acids can also include derivatives, variants and analogs of DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides can include deoxyadenosine, deoxycytidine, deoxyguanosine and deoxythymidine. A nucleic acid may be prepared using a nucleic acid obtained from a subject as a template.

In certain embodiments, the nucleic acid is a cell-free nucleic acid, which can be found in bodily fluids such as blood, whole blood, plasma, serum, urine, cerebrospinal fluid, fecal, saliva, sweat, sweat, tears, pleural fluid, pericardial fluid, or peritoneal fluid of a subject. In certain embodiments, a plasma sample can be used directly in the methods disclosed herein (for example, in the cutting step), without prior purification or isolation of nucleic acids in the plasma. Cell-free nucleic acids originate from one or more healthy cells and/or from one or more cancer cells, or from non-human sources such bacteria, fungi, viruses. Examples of the cell-free nucleic acids include but are not limited to cell-free DNA (“cfDNA”), including mitochondrial DNA or genomic DNA, and cell-free RNA. In certain embodiments herein, instruments for assessing the quality of the cell-free nucleic acids, such as the TapeStation System from Agilent Technologies (Santa Clara, CA) can be used. Concentrating low-abundance cfDNA can be accomplished, for example using a Qubit Fluorometer from Thermofisher Scientific (Waltham, MA).

In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free).

A methylated nucleic acid is a nucleic acid having a modification in which a hydrogen atom on the pyrimidine ring of a cytosine base is converted to a methyl group, forming 5-methylcytosine. Methylation can occur at dinucleotides of cytosine and guanine referred to herein as “CpG sites”, which can be a target for enrichment. Methylation of cytosine can occur in cytosines in other sequence contexts, for example, 5′-CHG-3′ and 5′-CHH-3′, where H is adenine, cytosine or thymine. Cytosine methylation can also be in the form of 5-hydroxymethylcytosine. Methylation of DNA can include methylation of non-cytosine nucleotides, such as N⁶-methyladenine (6 mA). Anomalous cfDNA methylation can be identified as hypermethylation or hypomethylation, both of which may be indicative of cancer status. As is well known in the art, DNA methylation anomalies (compared to healthy controls) can cause different effects, which may contribute to cancer.

In certain embodiments, the nucleic acid comprises a CpG site (i.e., cytosine and guanine separated by only one phosphate group). In certain embodiments, the nucleic acid comprises a CpG island (also referred to as a “CG islands” or “CGI”) or a portion thereof, which is the target for enrichment. Because certain CGIs and certain features of certain CGIs in tumor cells tend to be different from the same CGIs or features of the CGIs in healthy cells, detection of such CGIs can be informative of a health condition. In certain embodiments, the CGI is a “cancer informative CGIs”, which is defined and described in more detail below. In certain embodiments, the CpG is an “informative CpG”, e.g., a “cancer informative CGI”. Such CGIs may have methylation patterns in tumor cells that are different from the methylation patterns in healthy cells. Accordingly, detection of a cancer informative CGI can be informative regarding a subject's risk of developing cancer or can be indicative that the subject has cancer. Exemplary cancer informative CGIs, which can be target sequences as described herein, are identified in, e.g., Table 1 of U.S. Patent Publication 2020/0109456A1, Tables 2 and 3 of WO2022/133315, and Tables 1-4 provided herein.

In certain aspects, the nucleic acids of the invention have been treated to convert one or more unmethylated nucleotides (e.g., cytosines) to another nucleotide (a “converted nucleotide”, as used herein, such as a uracil), for example, prior to amplification. In certain embodiments, one or more unmethylated cytosines are converted to a nucleotide that pairs with adenine (e.g., the unmethylated cytosine may be converted to uracil). In certain embodiments, one or more unmethylated adenines are converted to a base that pairs with cytosine (e.g., the unmethylated adenine may be converted to inosine (I)). In certain embodiments, one or more methylated cytosines (e.g., a 5-methylcytosine (5 mC)) is converted to a thymine, which pairs with adenine. In certain embodiments, methylated cytosines are protected from conversion (e.g., deamination) during the conversion step.

After a nucleic acid has been treated to convert unmethylated, or, in some cases, methylated nucleotides, into another nucleotide, the nucleic acid may be amplified. During amplification, the converted nucleotide pairs with its complementary nucleotide, and in the next round of amplification, the complementary nucleotide pairs with a replacement nucleotide. For example, following the conversion of an unmethylated cytosine to a uracil, the nucleic acid may be amplified such that an adenine pairs with the uracil in the first round of replication, and in the second round of replication, the adenine pairs with a thymine. Accordingly, the thymine replaces the uracil in the original nucleic acid sequence, and is referred to herein as a “replacement nucleotide”.

### Bisulfite Conversion

Bisulfite conversion is performed on DNA by denaturation using high heat, preferential deamination (at an acidic pH) of unmethylated cytosines, which are then converted to uracil by desulfonation (at an alkaline pH). Methylated cytosines remain unchanged on the single-stranded DNA (ssDNA) product.

In some embodiments the methods include treatment of the sample with bisulfite (e.g., sodium bisulfite, potassium bisulfite, ammonium bisulfite, magnesium bisulfite, sodium metabisulfite, potassium metabisulfite, ammonium metabisulfite, magnesium metabisulfite and the like). Unmethylated cytosine is converted to uracil through a three-step process during sodium bisulfite modification. As shown in FIG. 2A, the steps are sulphonation to convert cytosine to cytosine sulphonate, deamination to convert cytosine sulphonate to uracil sulphonate and alkali desulphonation to convert uracil sulphonate to uracil. Conversion on methylated cytosine is much slower and is not observed at significant levels in a 4-16 hour reaction. (See Clark et al., Nucleic Acids Res., 22(15):2990-7 (1994).) If the cytosine is methylated it will remain a methylated cytosine. If the cytosine is unmethylated it will be converted to uracil. When the modified strand is copied, for example, through extension of a locus specific primer, a random or degenerate primer or a primer to an adaptor, a G will be incorporated in the interrogation position (opposite the C being interrogated) if the C was methylated and an A will be incorporated in the interrogation position if the C was unmethylated and converted to U. When the double stranded extension product is amplified those Cs that were converted to Us and resulted in incorporation of A in the extended primer will be replaced by Ts during amplification. Those Cs that were not converted (i.e., the methylated Cs) and resulted in the incorporation of G will be replaced by unmethylated Cs during amplification.

### Enzymatic Conversion

In certain embodiments, the enzymatic treatment with a cytidine deaminase enzyme is used to convert cytosine to uracil. Enzymatic conversion can include an oxidation step, in which Tet methylcytosine dioxygenase 2 (TET2) catalyzes the oxidation of 5 mC to 5 hmC to protect methylated cytosines from conversion by subsequent exposure to a cytidine deaminase. Other protection steps known in the art can be used in addition to or in place of oxidation by TET2. After the oxidation step, the nucleic acid is treated with the cytidine deaminase to convert one or more unmethylated cytosines to uracils. As with bisulfite conversion, when the modified strand is copied, a G will be incorporated in the interrogation position (opposite the C being interrogated) if the C was methylated and an A will be incorporated in the interrogation position if the C was unmethylated. When the double stranded extension product is amplified those Cs that were converted to Us and resulted in incorporation of A in the extended primer will be replaced by Ts during amplification. Those Cs that were not modified and resulted in the incorporation of G will remain as C.

In certain embodiments the cytidine deaminase may be APOBEC. In certain embodiments the cytidine deaminase includes activation induced cytidine deaminase (AID) and apolipoprotein B mRNA editing enzymes, catalytic polypeptide-like (APOBEC). In certain embodiments, the APOBEC enzyme is selected from the human APOBEC family consisting of: APOBEC-1 (Apo1), APOBEC-2 (Apo2), AID, APOBEC-3A, -3B, -3C, -3DE, -3F, -3G, -3H and APOBEC-4 (Apo4). In certain embodiments, the APOBEC enzyme is APOBEC-seq.

### Nitrite Conversion

In certain embodiments, nitrite treatment is used to deaminate adenine and cytosine. As shown in FIG. 2B, deamination of an A results in conversion to an inosine (I), which is read by a polymerase as a G, whereas deamination of a methylated A (N⁶-methyladenine (6 mA)) results in a nitrosylated 6 mA (6 mA-NO), which causes the base to be read by a polymerase as an A. Deamination of a C results in conversion to a uracil, which is read by a polymerase as a T, whereas deamination of a N⁴-methylcytosine (4 mC) to 4 mC-NO or a 5-methylcytosine (5 mC) to a T causes the base to be read by a polymerase as a C or a T, respectively. For 5 mC bases, the C to T ratio at the 5 mC position is about 40% higher than other cytosine positions, allowing 5 mC to be differentiated from C. (See, Li et al. (2022) Genome Biology 23:122.)

### Computer Implementation

Methods disclosed herein, including the methods of determining a signal informative for presence or absence of a cancer in a sample obtained from an individual, are, in some embodiments, performed on one or more computers. In particular embodiments, the method of determining a signal informative for presence or absence of a cancer in a sample obtained from an individual is performed on one or more computers.

In various embodiments, the method of determining a signal informative for presence or absence of a cancer in a sample obtained from an individual can be implemented in hardware or software, or a combination of both. In one embodiment, a machine-readable storage medium is provided, the medium comprising a data storage material encoded with machine readable data which, when using a machine programmed with instructions for using said data, is capable of displaying data and/or results. Methods disclosed herein can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

In some embodiments, methods disclosed herein, including methods for determining a signal informative for presence or absence of a cancer in a sample obtained from an individual, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

### Example Computer

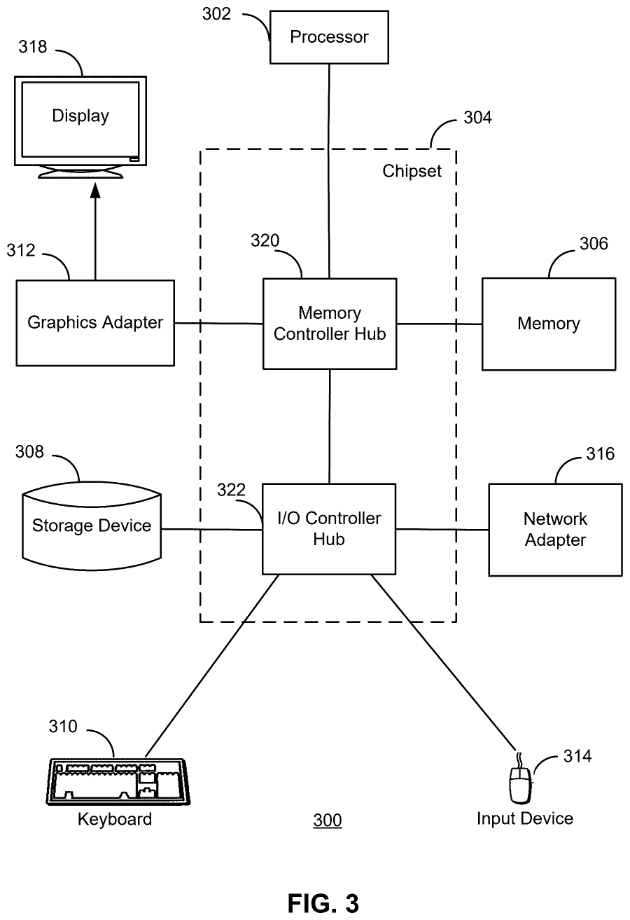

FIG. 3 illustrates an example computer for implementing methods in accordance with FIG. 1. The computer 300 includes at least one processor 302 coupled to a chipset 304. The chipset 304 includes a memory controller hub 320 and an input/output (I/O) controller hub 322. A memory 306 and a graphics adapter 312 are coupled to the memory controller hub 320, and a display 318 is coupled to the graphics adapter 312. A storage device 308, an input device 314, and network adapter 316 are coupled to the I/O controller hub 322. Other embodiments of the computer 300 have different architectures.

The storage device 308 is a non-transitory computer-readable storage medium such as a hard drive, compact disk read-only memory (CD-ROM), DVD, or a solid-state memory device. The memory 306 holds instructions and data used by the processor 302. The input interface 314 is a touch-screen interface, a mouse, track ball, or other type of pointing device, a keyboard, or some combination thereof, and is used to input data into the computer 300. In some embodiments, the computer 300 may be configured to receive input (e.g., commands) from the input interface 314 via gestures from the user. The graphics adapter 312 displays images and other information on the display 318. The network adapter 316 couples the computer 300 to one or more computer networks.

The computer 300 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 308, loaded into the memory 306, and executed by the processor 302. A module can be implemented as computer program code processed by the processing system(s) of one or more computers. Computer program code includes computer-executable instructions and/or computer-interpreted instructions, such as program modules, which instructions are processed by a processing system of a computer. Generally, such instructions define routines, programs, objects, components, data structures, and so on, that, when processed by a processing system, instruct the processing system to perform operations on data or configure the processor or computer to implement various components or data structures in computer storage. A data structure is defined in a computer program and specifies how data is organized in computer storage, such as in a memory device or a storage device, so that the data can accessed, manipulated, and stored by a processing system of a computer.

The types of computers 300 for performing methods disclosed herein can vary depending upon the embodiment and the processing power required by the entity. For example, methods can be performed on a single computer 300 or multiple computers 300 communicating with each other through a network such as in a server farm. The computers 300 can lack some of the components described above, such as graphics adapters 312, and displays 318.

TABLE 1
List of CGIs

| Reference | Pos (hg19 coordinates) |
|---|---|
| 1 | chr13: 108518334-108518633 |
| 2 | chr6: 137242315-137245442 |
| 3 | chr2: 177016416-177016632 |
| 4 | chr5: 2738953-2741237 |
| 5 | chr4: 111553079-111554210 |
| 6 | chr15: 96909815-96910030 |
| 7 | chr6: 42072032-42072701 |
| 8 | chr10: 123922850-123923542 |
| 9 | chr16: 86612188-86613821 |
| 10 | chr19: 47151768-47153125 |
| 11 | chr1: 110610265-110613303 |
| 12 | chr5: 3594467-3603054 |
| 13 | chr9: 126773246-126780953 |
| 14 | chr3: 138656627-138659107 |
| 15 | chr4: 4859632-4860191 |
| 16 | chr10: 118895963-118898037 |
| 17 | chr7: 103086344-103086840 |
| 18 | chr19: 407011-409511 |
| 19 | chr10: 22764708-22767050 |
| 20 | chr16: 86549069-86550512 |
| 21 | chr9: 96713326-96718186 |
| 22 | chr8: 139508795-139509774 |
| 23 | chr2: 73143055-73148260 |
| 24 | chr8: 26721642-26724566 |
| 25 | chr9: 129386112-129389231 |
| 26 | chr12: 49483601-49484255 |
| 27 | chr16: 54325040-54325703 |
| 28 | chr8: 72468560-72469561 |
| 29 | chr18: 70533965-70536871 |
| 30 | chr9: 98111364-98112362 |
| 31 | chr1: 50882997-50883426 |
| 32 | chr10: 88122924-88127364 |
| 33 | chr11: 31839363-31839813 |
| 34 | chr10: 101290025-101290338 |
| 35 | chr6: 41528266-41528900 |
| 36 | chr16: 51183699-51188763 |
| 37 | chr5: 140346105-140346931 |
| 38 | chr9: 23820691-23822135 |
| 39 | chr20: 690575-691099 |
| 40 | chr1: 177133392-177133846 |
| 41 | chr5: 45695394-45696510 |
| 42 | chr2: 45395869-45398186 |
| 43 | chr20: 48184193-48184833 |
| 44 | chr6: 6002471-6005125 |
| 45 | chr14: 101192851-101193499 |
| 46 | chr8: 4848968-4852635 |
| 47 | chr8: 53851701-53854426 |
| 48 | chr12: 186863-187610 |
| 49 | chr5: 54519054-54519628 |
| 50 | chr6: 108485671-108490539 |
| 51 | chr3: 157815581-157816095 |
| 52 | chr11: 626728-628037 |
| 53 | chr2: 177012371-177012675 |
| 54 | chr17: 59531723-59535254 |
| 55 | chr16: 55364823-55365483 |
| 56 | chr8: 99960497-99961438 |
| 57 | chr7: 42267546-42267823 |
| 58 | chr17: 14202632-14203258 |
| 59 | chr10: 102891010-102891794 |
| 60 | chr5: 174158680-174159729 |
| 61 | chr14: 33402094-33404079 |
| 62 | chr2: 177036254-177037213 |
| 63 | chr10: 106399567-106402812 |
| 64 | chr6: 166579973-166583423 |
| 65 | chr11: 123066517-123066986 |
| 66 | chr11: 44327240-44327932 |
| 67 | chr14: 95237622-95238211 |
| 68 | chr9: 102590742-102591303 |
| 69 | chr15: 76630029-76630970 |
| 70 | chr4: 24801109-24801902 |
| 71 | chr8: 97169731-97170432 |
| 72 | chr3: 6902823-6903516 |
| 73 | chr22: 48884884-48887043 |
| 74 | chr15: 45408573-45409528 |
| 75 | chr9: 100610696-100611517 |
| 76 | chr4: 174448333-174448845 |
| 77 | chr16: 20084707-20085305 |
| 78 | chr4: 174439812-174440249 |
| 79 | chr6: 10381558-10382354 |
| 80 | chr15: 35046443-35047480 |
| 81 | chr10: 119494493-119494991 |
| 82 | chr5: 72676120-72678421 |
| 83 | chr11: 44325657-44326517 |
| 84 | chr17: 46670522-46671458 |
| 85 | chr14: 92789494-92790712 |
| 86 | chr4: 174459200-174460054 |
| 87 | chr2: 80549578-80549798 |
| 88 | chr7: 153748407-153750444 |
| 89 | chr6: 1389139-1391393 |
| 90 | chr16: 49314037-49316543 |
| 91 | chr2: 105459127-105461770 |
| 92 | chr21: 38079941-38081833 |
| 93 | chr4: 174427891-174428192 |
| 94 | chr14: 60973772-60974123 |
| 95 | chr8: 99985733-99986983 |
| 96 | chr2: 63281034-63281347 |
| 97 | chr12: 101109863-101111622 |
| 98 | chr1: 119549144-119551320 |
| 99 | chr5: 38257825-38259136 |
| 100 | chr5: 54522302-54523533 |
| 101 | chr1: 165324191-165326328 |
| 102 | chr15: 33602816-33604003 |
| 103 | chr10: 118030732-118034230 |
| 104 | chr2: 45240372-45241579 |
| 105 | chr4: 174430386-174430861 |
| 106 | chr6: 50810642-50810994 |
| 107 | chr5: 122430676-122431443 |
| 108 | chr10: 109674196-109674964 |
| 109 | chr8: 97172634-97173880 |
| 110 | chr8: 11536767-11538961 |
| 111 | chr5: 180486154-180486892 |
| 112 | chr2: 38301276-38304518 |
| 113 | chr10: 1778784-1780018 |
| 114 | chr12: 54424610-54425173 |
| 115 | chr17: 46669434-46669811 |
| 116 | chr11: 8190226-8190671 |
| 117 | chr8: 25900562-25905842 |
| 118 | chr12: 81102034-81102716 |
| 119 | chr7: 27199661-27200960 |
| 120 | chr10: 119311204-119312104 |
| 121 | chr12: 130387609-130389139 |
| 122 | chr7: 155258827-155261403 |
| 123 | chr6: 117591533-117592279 |
| 124 | chr10: 111216604-111217083 |
| 125 | chr1: 29585897-29586598 |
| 126 | chr2: 144694666-144695180 |
| 127 | chr12: 48397889-48398731 |
| 128 | chr5: 2748368-2757024 |
| 129 | chr12: 114845861-114847650 |
| 130 | chr2: 80529677-80530846 |
| 131 | chr5: 1874907-1879032 |
| 132 | chr6: 100905952-100906686 |
| 133 | chr15: 96904722-96905050 |
| 134 | chr5: 134374385-134376751 |
| 135 | chr2: 66652691-66654218 |
| 136 | chr12: 54440642-54441543 |
| 137 | chr6: 108495654-108495986 |
| 138 | chr17: 70112824-70114271 |
| 139 | chr3: 87841796-87842563 |
| 140 | chr7: 96650221-96651551 |
| 141 | chr4: 110222970-110224257 |
| 142 | chr6: 78172231-78174088 |
| 143 | chr7: 155164557-155167854 |
| 144 | chr12: 113900750-113906442 |
| 145 | chr9: 112081402-112082905 |
| 146 | chr12: 114886354-114886579 |
| 147 | chr5: 3590644-3592000 |
| 148 | chr2: 119592602-119593845 |
| 149 | chr20: 21485932-21496714 |
| 150 | chr18: 11148307-11149936 |
| 151 | chr17: 46824785-46825372 |
| 152 | chr10: 100992156-100992687 |
| 153 | chr14: 36986362-36990576 |
| 154 | chr18: 55094825-55096310 |
| 155 | chr15: 96895306-96895729 |
| 156 | chr17: 36717727-36718593 |
| 157 | chr2: 223183013-223185468 |
| 158 | chr7: 30721372-30722445 |
| 159 | chr1: 53527572-53528974 |
| 160 | chr18: 56939624-56941540 |
| 161 | chr5: 175085004-175085756 |
| 162 | chr10: 50817601-50820356 |
| 163 | chr14: 60975732-60978180 |
| 164 | chr15: 89920793-89922768 |
| 165 | chr9: 122131086-122132214 |
| 166 | chr1: 217311467-217311773 |
| 167 | chr 14: 38724254-38725537 |
| 168 | chr14: 61103978-61104663 |
| 169 | chr18: 73167402-73167920 |
| 170 | chr1: 50880916-50881516 |
| 171 | chr2: 241758141-241760783 |
| 172 | chr11: 31825743-31826967 |
| 173 | chr7: 27260101-27260467 |
| 174 | chr20: 41817475-41819212 |
| 175 | chr3: 238391-240140 |
| 176 | chr7: 121950249-121950927 |
| 177 | chr5: 72526203-72526497 |
| 178 | chr15: 96903311-96903711 |
| 179 | chr10: 26504383-26507434 |
| 180 | chr6: 100915602-100915883 |
| 181 | chr1: 18962842-18963481 |
| 182 | chr3: 127794369-127796136 |
| 183 | chr7: 27203915-27206462 |
| 184 | chr8: 25899335-25899692 |
| 185 | chr12: 114838312-114838889 |
| 186 | chr6: 38682949-38683265 |
| 187 | chr11: 31841315-31842003 |
| 188 | chr4: 174451828-174452962 |
| 189 | chr9: 129372737-129378106 |
| 190 | chr2: 176964062-176965509 |
| 191 | chr2: 176931575-176932663 |
| 192 | chr12: 114833911-114834210 |
| 193 | chr11: 79148358-79152200 |
| 194 | chr2: 177024501-177025692 |
| 195 | chr5: 172672311-172672971 |
| 196 | chr7: 27291119-27292197 |
| 197 | chr1: 180198119-180204975 |
| 198 | chr14: 37126786-37128274 |
| 199 | chr2: 200333687-200334172 |
| 200 | chr14: 58331676-58333121 |
| 201 | chr3: 147131066-147131333 |
| 202 | chr13: 109147798-109149019 |
| 203 | chr14: 48143433-48145589 |
| 204 | chr6: 100905444-100905697 |
| 205 | chr17: 14200579-14200996 |
| 206 | chr6: 1379693-1380014 |
| 207 | chr1: 34642382-34643024 |
| 208 | chr2: 119599059-119599299 |
| 209 | chr2: 119613031-119615565 |
| 210 | chr4: 85413997-85414874 |
| 211 | chr9: 17906419-17907488 |
| 212 | chr12: 29302034-29302954 |
| 213 | chr20: 10200088-10200384 |
| 214 | chr8: 57358126-57359415 |
| 215 | chr10: 63212495-63213009 |
| 216 | chr2: 176936246-176936809 |
| 217 | chr11: 20618197-20619920 |
| 218 | chr18: 19744936-19752363 |
| 219 | chr14: 29234889-29235908 |
| 220 | chr17: 46673532-46674181 |
| 221 | chr4: 144620822-144622218 |
| 222 | chr16: 82660651-82661813 |
| 223 | chr3: 192125821-192127994 |
| 224 | chr2: 119599458-119600966 |
| 225 | chr22: 44257942-44258612 |
| 226 | chr19: 13616752-13617267 |
| 227 | chr3: 147138916-147139564 |
| 228 | chr9: 969529-973276 |
| 229 | chr18: 55103154-55108853 |
| 230 | chr4: 174422024-174422443 |
| 231 | chr4: 57521621-57522703 |
| 232 | chr15: 79724099-79725643 |
| 233 | chr14: 37135513-37136348 |
| 234 | chr10: 23480697-23482455 |
| 235 | chr2: 45169505-45171884 |
| 236 | chr18: 30349690-30352302 |
| 237 | chr6: 99291327-99291737 |
| 238 | chr9: 21970913-21971190 |
| 239 | chr4: 107146-107898 |
| 240 | chr12: 117798076-117799448 |
| 241 | chr2: 219736132-219736592 |
| 242 | chr10: 118892161-118892639 |
| 243 | chr11: 27743472-27744564 |
| 244 | chr12: 65218245-65219143 |
| 245 | chr12: 75601081-75601752 |
| 246 | chr7: 54612324-54612558 |
| 247 | chr6: 100912071-100913337 |
| 248 | chr10: 102905714-102906693 |
| 249 | chr8: 87081653-87082046 |
| 250 | chr6: 50818180-50818431 |
| 251 | chr1: 91189139-91189400 |
| 252 | chr2: 118981769-118982466 |
| 253 | chr10: 50602989-50606783 |
| 254 | chr17: 59528979-59530266 |
| 255 | chr4: 147559205-147561901 |
| 256 | chr1: 4713989-4716555 |
| 257 | chr13: 102568425-102569495 |
| 258 | chr16: 6068914-6070401 |
| 259 | chr22: 29709281-29712013 |
| 260 | chr10: 100993820-100994188 |
| 261 | chr6: 391188-393790 |
| 262 | chr2: 176977284-176977540 |
| 263 | chr4: 4868440-4869173 |
| 264 | chr6: 137809342-137810204 |
| 265 | chr12: 54321301-54321721 |
| 266 | chr2: 105468851-105473488 |
| 267 | chr8: 55366180-55367628 |
| 268 | chr12: 72665683-72667551 |
| 269 | chr4: 54966163-54968063 |
| 270 | chr5: 134366913-134367438 |
| 271 | chr1: 226075150-226075680 |
| 272 | chr20: 17206528-17206952 |
| 273 | chr4: 172733734-172735118 |
| 274 | chr18: 55019707-55021605 |
| 275 | chr2: 162279835-162280709 |
| 276 | chr6: 1381743-1385211 |
| 277 | chr7: 103968783-103969959 |
| 278 | chr6: 150358872-150359394 |
| 279 | chr2: 119914126-119916663 |
| 280 | chr7: 27278945-27279469 |
| 281 | chr12: 114851957-114852360 |
| 282 | chr16: 24267040-24267527 |
| 283 | chr6: 7229877-7230865 |
| 284 | chr2: 45227644-45228783 |
| 285 | chr4: 174450046-174451469 |
| 286 | chr4: 154712073-154712706 |
| 287 | chr3: 22413492-22414365 |
| 288 | chr20: 21694472-21695344 |
| 289 | chr6: 1378445-1379318 |
| 290 | chr8: 70981873-70984888 |
| 291 | chr12: 53107912-53108471 |
| 292 | chr10: 102996034-102996646 |
| 293 | chr3: 157821232-157821604 |
| 294 | chr4: 111554965-111555504 |
| 295 | chr13: 58206526-58208930 |
| 296 | chr10: 22634000-22634862 |
| 297 | chr9: 22005887-22006229 |
| 298 | chr5: 159399004-159399928 |
| 299 | chr2: 31805293-31806403 |
| 300 | chr6: 100903491-100903713 |
| 301 | chr5: 77268350-77268787 |
| 302 | chr14: 85997468-85998637 |
| 303 | chr5: 92923487-92924497 |
| 304 | chr11: 64480199-64481344 |
| 305 | chr13: 28366549-28368505 |
| 306 | chr5: 77805753-77806313 |
| 307 | chr9: 79633326-79636030 |
| 308 | chr4: 93226348-93227007 |
| 309 | chr2: 223170486-223171140 |
| 310 | chr1: 91172102-91172771 |
| 311 | chr1: 1181756-1182470 |
| 312 | chr8: 65281903-65283043 |
| 313 | chr10: 94825546-94826320 |
| 314 | chr6: 108491033-108491410 |
| 315 | chr21: 38076762-38077685 |
| 316 | chr1: 91183240-91184540 |
| 317 | chr3: 147136903-147137328 |
| 318 | chr15: 96911511-96911808 |
| 319 | chr14: 57274607-57276840 |
| 320 | chr13: 112726281-112728419 |
| 321 | chr2: 171672310-171675447 |
| 322 | chr8: 11559596-11562956 |
| 323 | chr10: 48438411-48439320 |
| 324 | chr18: 59000683-59001692 |
| 325 | chr15: 91642908-91643702 |
| 326 | chr5: 3592391-3592644 |
| 327 | chr19: 56988313-56989741 |
| 328 | chr6: 26614013-26614851 |
| 329 | chr11: 27742059-27742273 |
| 330 | chr3: 147113608-147114479 |
| 331 | chr14: 57264638-57265561 |
| 332 | chr7: 155302253-155303158 |
| 333 | chr11: 31848487-31848776 |
| 334 | chr16: 54970301-54972846 |
| 335 | chr19: 30715549-30715753 |
| 336 | chr9: 96710811-96711717 |
| 337 | chr18: 77557780-77558948 |
| 338 | chr20: 21686199-21687689 |
| 339 | chr11: 31847132-31847958 |
| 340 | chr16: 86530747-86532994 |
| 341 | chr1: 203044722-203045390 |
| 342 | chr15: 53096014-53096482 |
| 343 | chr7: 97361132-97363018 |
| 344 | chr14: 29236835-29237832 |
| 345 | chr13: 79182859-79183880 |
| 346 | chr11: 69517840-69519929 |
| 347 | chr1: 231296559-231297345 |
| 348 | chr19: 8675333-8675699 |
| 349 | chr1: 63795363-63796140 |
| 350 | chr4: 90228714-90229010 |
| 351 | chr3: 62362610-62363082 |
| 352 | chr19: 5827754-5828405 |
| 353 | chr10: 125732220-125732843 |
| 354 | chr9: 136293566-136294160 |
| 355 | chr1: 63782394-63790471 |
| 356 | chr4: 4867386-4867673 |
| 357 | chr9: 133534534-133542394 |
| 358 | chr15: 100913438-100914022 |
| 359 | chr10: 101279941-101280382 |
| 360 | chr13: 53419897-53422872 |
| 361 | chr1: 77747314-77748224 |
| 362 | chr14: 36974548-36975425 |
| 363 | chr12: 57618769-57619402 |
| 364 | chr7: 49813008-49815752 |
| 365 | chr4: 188916605-188916876 |
| 366 | chr11: 31831620-31839038 |
| 367 | chr8: 132052203-132054749 |
| 368 | chr2: 237071794-237078762 |
| 369 | chr20: 39994545-39995810 |
| 370 | chr11: 132812662-132813075 |
| 371 | chr5: 170735169-170739863 |
| 372 | chr1: 221051966-221053673 |
| 373 | chr5: 72529099-72529976 |
| 374 | chr14: 36973169-36973740 |
| 375 | chr4: 158141404-158141836 |
| 376 | chr14: 103655241-103655928 |
| 377 | chr1: 65731411-65731849 |
| 378 | chr1: 38218190-38218977 |
| 379 | chr3: 128719865-128721245 |
| 380 | chr15: 33009530-33011696 |
| 381 | chr2: 162275161-162275596 |
| 382 | chr7: 155241323-155243757 |
| 383 | chr19: 46001830-46002686 |
| 384 | chr6: 137814355-137815202 |
| 385 | chr7: 70596228-70598382 |
| 386 | chr15: 96959341-96960531 |
| 387 | chr16: 66612749-66613412 |
| 388 | chr6: 110299365-110301267 |
| 389 | chr15: 27215951-27216856 |
| 390 | chr11: 88241710-88242562 |
| 391 | chr2: 124782252-124783255 |
| 392 | chr17: 70111979-70112308 |
| 393 | chr2: 63283936-63284147 |
| 394 | chr17: 46800945-46801288 |
| 395 | chr6: 1393049-1394170 |
| 396 | chr3: 137489594-137491004 |
| 397 | chr15: 60296135-60298520 |
| 398 | chr12: 106979429-106981086 |
| 399 | chr12: 54360374-54360660 |
| 400 | chr 14: 36991594-36992488 |
| 401 | chr4: 156129168-156130209 |
| 402 | chr4: 54975387-54976202 |
| 403 | chr3: 137482964-137484454 |
| 404 | chr10: 118893527-118894432 |
| 405 | chr18: 76737005-76741244 |
| 406 | chr10: 110671724-110672326 |
| 407 | chr5: 71014917-71015715 |
| 408 | chr6: 50787286-50788091 |
| 409 | chr19: 3868586-3869217 |
| 410 | chr4: 5894071-5895116 |
| 411 | chr11: 131780328-131781532 |
| 412 | chr6: 101846766-101847135 |
| 413 | chr11: 71952112-71952528 |
| 414 | chr5: 172663616-172664584 |
| 415 | chr9: 23822412-23822667 |
| 416 | chr4: 5891981-5892365 |
| 417 | chr1: 217310749-217311178 |
| 418 | chr10: 108923780-108924805 |
| 419 | chr6: 100038655-100039477 |
| 420 | chr7: 121945345-121946235 |
| 421 | chr3: 147126988-147128999 |
| 422 | chr7: 121956543-121957341 |
| 423 | chr4: 156680095-156681386 |
| 424 | chr4: 85404986-85405252 |
| 425 | chr1: 221064889-221065600 |
| 426 | chr17: 73749618-73750178 |
| 427 | chr8: 55370170-55372525 |
| 428 | chr6: 70992040-70992912 |
| 429 | chr16: 55513220-55513526 |
| 430 | chr6: 106433984-106434459 |
| 431 | chr14: 29254365-29255069 |
| 432 | chr6: 33655966-33656238 |
| 433 | chr9: 19788215-19789288 |
| 434 | chr11: 115630398-115631117 |
| 435 | chr1: 34628783-34630976 |
| 436 | chr14: 101923575-101925995 |
| 437 | chr17: 72855621-72858012 |
| 438 | chr2: 223162946-223163912 |
| 439 | chr4: 85417659-85420799 |
| 440 | chr1: 156390403-156391581 |
| 441 | chr3: 147130342-147130577 |
| 442 | chr2: 119602616-119604486 |
| 443 | chr9: 120175253-120177496 |
| 444 | chr4: 174443365-174443948 |
| 445 | chr5: 145724294-145724551 |
| 446 | chr11: 32454874-32457311 |
| 447 | chr2: 176949511-176949795 |
| 448 | chr1: 18436551-18437673 |
| 449 | chr3: 26665950-26666164 |
| 450 | chr3: 170303044-170303249 |
| 451 | chr2: 223176493-223177515 |
| 452 | chr2: 182321761-182323029 |
| 453 | chr18: 44789742-44790678 |
| 454 | chr17: 46796234-46797292 |
| 455 | chr18: 44772992-44775577 |
| 456 | chr8: 101117922-101118693 |
| 457 | chr7: 27134097-27134303 |
| 458 | chr10: 102507482-102509646 |
| 459 | chr19: 39754973-39756540 |
| 460 | chr7: 26415746-26416891 |
| 461 | chr14: 37116188-37117628 |
| 462 | chr4: 174421347-174421559 |
| 463 | chr6: 85472702-85474132 |
| 464 | chr20: 22557517-22559240 |
| 465 | chr6: 117198089-117198705 |
| 466 | chr10: 71331926-71333392 |
| 467 | chr19: 36334994-36335321 |
| 468 | chr4: 46995128-46995872 |
| 469 | chr9: 135455164-135458586 |
| 470 | chr8: 65290108-65290946 |
| 471 | chr10: 94828102-94829040 |
| 472 | chr1: 116380359-116382364 |
| 473 | chr15: 47476369-47477499 |
| 474 | chr3: 147115764-147116421 |
| 475 | chr17: 59485573-59485780 |
| 476 | chr10: 23983366-23984978 |
| 477 | chr2: 176949993-176950336 |
| 478 | chr9: 137967110-137967727 |
| 479 | chr2: 176957054-176958279 |
| 480 | chr11: 119293320-119293943 |
| 481 | chr11: 132813562-132814395 |
| 482 | chr2: 237068071-237068834 |
| 483 | chr10: 27547668-27548402 |
| 484 | chr4: 4866438-4866813 |
| 485 | chr21: 19617098-19617874 |
| 486 | chr1: 91185156-91185577 |
| 487 | chr19: 15292399-15292632 |
| 488 | chr1: 145075483-145075845 |
| 489 | chr2: 19560963-19561650 |
| 490 | chr14: 57260878-57262123 |
| 491 | chr8: 55378928-55380186 |
| 492 | chr6: 99290279-99290771 |
| 493 | chr19: 13124959-13125259 |
| 494 | chr15: 27112030-27113479 |
| 495 | chr8: 145925410-145926101 |
| 496 | chr11: 124629723-124629926 |
| 497 | chr4: 109093038-109094546 |
| 498 | chr3: 62356773-62357315 |
| 499 | chr14: 37131181-37132785 |
| 500 | chr10: 124905634-124906161 |
| 501 | chr7: 35296921-35298218 |
| 502 | chr19: 36248979-36249307 |
| 503 | chr12: 15475318-15475901 |
| 504 | chr5: 87985470-87985810 |
| 505 | chr12: 54423427-54423712 |
| 506 | chr7: 96653467-96654199 |
| 507 | chr2: 45155195-45157049 |
| 508 | chr15: 96896928-96897301 |
| 509 | chr12: 58004982-58005351 |
| 510 | chr2: 176933131-176933449 |
| 511 | chr2: 176962179-176962487 |
| 512 | chr20: 25063838-25065525 |
| 513 | chr12: 5153012-5154346 |
| 514 | chr3: 154146347-154146965 |
| 515 | chr1: 165323486-165323811 |
| 516 | chr21: 38065179-38066185 |
| 517 | chr10: 119000435-119001530 |
| 518 | chr12: 45444202-45445386 |
| 519 | chr4: 158143296-158144053 |
| 520 | chr5: 76932317-76933523 |
| 521 | chr5: 172659049-172660277 |
| 522 | chr2: 223168653-223169008 |
| 523 | chr1: 248020330-248021252 |
| 524 | chr18: 904578-909574 |
| 525 | chr12: 127940451-127940907 |
| 526 | chr9: 135461934-135462909 |
| 527 | chr17: 48041282-48043064 |
| 528 | chr4: 94755786-94756310 |
| 529 | chr10: 130338695-130338994 |
| 530 | chr2: 119616133-119616826 |
| 531 | chr2: 177042751-177043444 |
| 532 | chr2: 105478600-105479188 |
| 533 | chr5: 172670829-172671824 |
| 534 | chr2: 176952695-176953297 |
| 535 | chr13: 28549839-28550246 |
| 536 | chr13: 112720564-112723582 |
| 537 | chr6: 100895773-100896062 |
| 538 | chr7: 136553854-136556194 |
| 539 | chr6: 127441553-127441760 |
| 540 | chr1: 119526782-119527192 |
| 541 | chr12: 49484920-49485178 |
| 542 | chr9: 23850910-23851522 |
| 543 | chr2: 220299483-220300243 |
| 544 | chr5: 1881924-1887743 |
| 545 | chr8: 57360585-57360815 |
| 546 | chr18: 74961556-74963822 |
| 547 | chr5: 172660720-172661133 |
| 548 | chr17: 75277317-75278172 |
| 549 | chr10: 99789614-99791320 |
| 550 | chr2: 176944087-176948446 |
| 551 | chr4: 154709512-154710827 |
| 552 | chr5: 140798757-140799359 |
| 553 | chr3: 44063314-44063837 |
| 554 | chr15: 79574830-79575211 |
| 555 | chr2: 223161531-223161919 |
| 556 | chr6: 134210639-134211218 |
| 557 | chr10: 102899177-102899489 |
| 558 | chr13: 79181944-79182222 |
| 559 | chr7: 71800757-71802768 |
| 560 | chr3: 186078710-186080111 |
| 561 | chr1: 24229115-24229537 |
| 562 | chr16: 48844551-48845264 |
| 563 | chr7: 113724924-113727795 |
| 564 | chr22: 44726724-44727590 |
| 565 | chr4: 15779998-15780729 |
| 566 | chr4: 41869174-41869459 |
| 567 | chr1: 38941919-38942404 |
| 568 | chr2: 176971706-176972305 |
| 569 | chr2: 119607378-119607910 |
| 570 | chr5: 76934581-76935296 |
| 571 | chr12: 103696090-103696418 |
| 572 | chr5: 63255044-63255407 |
| 573 | chr1: 221067447-221068185 |
| 574 | chr2: 119611296-119611881 |
| 575 | chr10: 124907283-124911035 |
| 576 | chr12: 114878143-114879155 |
| 577 | chr12: 49371690-49375550 |
| 578 | chr17: 36719544-36719938 |
| 579 | chr17: 46696553-46696926 |
| 580 | chr3: 147142181-147142391 |
| 581 | chr8: 9762661-9764748 |
| 582 | chr14: 74706188-74708192 |
| 583 | chr3: 12837992-12838359 |
| 584 | chr20: 37352130-37357372 |
| 585 | chr10: 8077829-8078378 |
| 586 | chr4: 4864456-4864834 |
| 587 | chr4: 13524062-13526083 |
| 588 | chr1: 66258440-66258918 |
| 589 | chr11: 17740789-17743779 |
| 590 | chr12: 106975195-106975714 |
| 591 | chr9: 91792662-91793611 |
| 592 | chr1: 149333785-149334111 |
| 593 | chr3: 170303532-170303768 |
| 594 | chr5: 72594147-72595808 |
| 595 | chr5: 145725286-145725852 |
| 596 | chr10: 23462224-23463889 |
| 597 | chr20: 21689758-21690048 |
| 598 | chr15: 53080458-53083699 |
| 599 | chr2: 154727906-154728271 |
| 600 | chr5: 170743178-170744107 |
| 601 | chr10: 102899822-102900263 |
| 602 | chr5: 134368578-134370466 |
| 603 | chr2: 66808568-66809404 |
| 604 | chr7: 96651963-96652246 |
| 605 | chr1: 91190489-91192804 |
| 606 | chr17: 75368688-75370506 |
| 607 | chr4: 185939222-185942747 |
| 608 | chr7: 43152020-43153340 |
| 609 | chr13: 84453664-84453897 |
| 610 | chr2: 176956504-176956707 |
| 611 | chr7: 87563342-87564571 |
| 612 | chr20: 17208550-17208756 |
| 613 | chr22: 19746924-19747141 |
| 614 | chr2: 223159725-223160487 |
| 615 | chr12: 131200509-131200726 |
| 616 | chr18: 44336183-44337110 |
| 617 | chr2: 63285949-63287097 |
| 618 | chr4: 13526553-13526770 |
| 619 | chr15: 89949373-89951130 |
| 620 | chr19: 55815940-55816277 |
| 621 | chr17: 50235175-50236466 |
| 622 | chr19: 58545115-58545897 |
| 623 | chr12: 113592203-113592620 |
| 624 | chr12: 115109503-115110061 |
| 625 | chr4: 164264821-164265772 |
| 626 | chr1: 2772126-2772665 |
| 627 | chr3: 71834068-71834653 |
| 628 | chr12: 5018585-5021171 |
| 629 | chr15: 74419870-74423044 |
| 630 | chr3: 147108511-147111703 |
| 631 | chr5: 88185224-88185589 |
| 632 | chr12: 54354529-54355491 |
| 633 | chr10: 101290625-101291178 |
| 634 | chr8: 11557852-11558252 |
| 635 | chr8: 105478672-105479340 |
| 636 | chr11: 20181200-20182325 |
| 637 | chr19: 54483021-54483572 |
| 638 | chr13: 112707804-112708696 |
| 639 | chr16: 22824616-22826459 |
| 640 | chr4: 66536065-66536674 |
| 641 | chr4: 154713537-154714240 |
| 642 | chr7: 12151220-12151559 |
| 643 | chr12: 119212110-119212393 |
| 644 | chr17: 14201726-14202052 |
| 645 | chr20: 21376358-21378245 |
| 646 | chr13: 36045931-36046143 |
| 647 | chr15: 60287107-60287663 |
| 648 | chr9: 100613938-100614622 |
| 649 | chr10: 102475276-102475579 |
| 650 | chr7: 121940006-121940648 |
| 651 | chr5: 37834671-37835128 |
| 652 | chr1: 197887088-197887791 |
| 653 | chr12: 99139386-99139769 |
| 654 | chr6: 1619093-1621094 |
| 655 | chr12: 113917394-113918107 |
| 656 | chr14: 24044886-24046760 |
| 657 | chr5: 77253832-77254049 |
| 658 | chr4: 85403830-85404524 |
| 659 | chr6: 166666837-166667541 |
| 660 | chr18: 77547965-77549038 |
| 661 | chr2: 219848919-219850541 |
| 662 | chr17: 7832532-7833164 |
| 663 | chr5: 134363092-134365146 |
| 664 | chr10: 103043990-103044480 |
| 665 | chr8: 97171805-97172022 |
| 666 | chr20: 57089460-57090237 |
| 667 | chr12: 114840853-114841063 |
| 668 | chr4: 66535193-66535620 |
| 669 | chr8: 85096759-85097247 |
| 670 | chr6: 10881846-10882051 |
| 671 | chr13: 28498226-28499046 |
| 672 | chr1: 161695637-161697298 |
| 673 | chr11: 2890388-2891337 |
| 674 | chr17: 5000369-5001205 |
| 675 | chr13: 27334226-27335205 |
| 676 | chr10: 22623350-22625875 |
| 677 | chr2: 157185557-157186355 |
| 678 | chr7: 20370003-20371504 |
| 679 | chr4: 961347-962155 |
| 680 | chr12: 49485766-49485977 |
| 681 | chr3: 62356119-62356378 |
| 682 | chr11: 14995128-14995908 |
| 683 | chr12: 53359192-53359507 |
| 684 | chr16: 51168266-51169110 |
| 685 | chr14: 57278709-57279116 |
| 686 | chr6: 37616722-37617179 |
| 687 | chr18: 11750953-11752756 |
| 688 | chr19: 45260352-45261809 |
| 689 | chr1: 119531991-119532196 |
| 690 | chr19: 36523391-36523887 |
| 691 | chr12: 52652018-52652743 |
| 692 | chr8: 49468683-49468959 |
| 693 | chr8: 9760750-9761643 |
| 694 | chr7: 19146923-19147308 |
| 695 | chr13: 32889533-32889900 |
| 696 | chr5: 140797162-140797701 |
| 697 | chr21: 42218489-42219222 |
| 698 | chr19: 54411376-54411968 |
| 699 | chr3: 62354291-62355012 |
| 700 | chr12: 113590806-113591304 |
| 701 | chr1: 225865068-225865328 |
| 702 | chr7: 130790358-130792773 |
| 703 | chr15: 53076187-53077926 |
| 704 | chr1: 214158726-214159080 |
| 705 | chr12: 3308812-3310270 |
| 706 | chr1: 39044059-39044561 |
| 707 | chr10: 119312766-119313563 |
| 708 | chr12: 65514878-65515863 |
| 709 | chr12: 54366815-54369103 |
| 710 | chr12: 114885105-114885418 |
| 711 | chr16: 2228190-2230946 |
| 712 | chr11: 68622722-68623252 |
| 713 | chr2: 25499763-25500429 |
| 714 | chr5: 172661486-172662228 |
| 715 | chr17: 46691520-46692097 |
| 716 | chr12: 75602991-75603344 |
| 717 | chr2: 80531367-80531719 |
| 718 | chr5: 158478378-158478630 |
| 719 | chr2: 177017266-177017489 |
| 720 | chr2: 63282514-63283122 |
| 721 | chr7: 155595692-155599414 |
| 722 | chr5: 172665306-172666072 |
| 723 | chr12: 114843022-114843610 |
| 724 | chr13: 112758598-112760491 |
| 725 | chr4: 4858389-4858893 |
| 726 | chr16: 55365814-55366022 |
| 727 | chr9: 96108466-96108992 |
| 728 | chr12: 3475010-3475654 |
| 729 | chr9: 86152353-86153777 |
| 730 | chr6: 10384965-10385492 |
| 731 | chr22: 31500396-31501239 |
| 732 | chr5: 179228283-179229003 |
| 733 | chr6: 137816474-137817223 |
| 734 | chr2: 106681982-106682403 |
| 735 | chr14: 95239375-95239679 |
| 736 | chr7: 154001964-154002281 |
| 737 | chr1: 1476093-1476669 |
| 738 | chr15: 89904822-89906050 |
| 739 | chr11: 89224416-89224718 |
| 740 | chr9: 100615234-100617510 |
| 741 | chr3: 172165372-172166738 |
| 742 | chr1: 202678881-202679769 |
| 743 | chr14: 37053134-37053690 |
| 744 | chr4: 41875445-41875794 |
| 745 | chr2: 162273294-162273725 |
| 746 | chr1: 181287300-181287873 |
| 747 | chr13: 79181327-79181614 |
| 748 | chr8: 145103285-145108027 |
| 749 | chr22: 42305617-42307254 |
| 750 | chr8: 102505512-102506430 |
| 751 | chr17: 74533281-74534566 |
| 752 | chr1: 214156000-214156851 |
| 753 | chr20: 2780978-2781497 |
| 754 | chr4: 4861227-4862241 |
| 755 | chr19: 13215244-13215543 |
| 756 | chr7: 121943867-121944538 |
| 757 | chr17: 71948478-71949255 |
| 758 | chr2: 127413696-127414171 |
| 759 | chr1: 113286332-113287172 |
| 760 | chr1: 47009575-47010132 |
| 761 | chr16: 62069121-62070634 |
| 762 | chr16: 3013651-3015131 |
| 763 | chr18: 76732970-76734765 |
| 764 | chr4: 155664819-155665833 |
| 765 | chr6: 72298274-72298528 |
| 766 | chr15: 89147660-89149198 |
| 767 | chr17: 33775294-33775794 |
| 768 | chr18: 44337510-44338100 |
| 769 | chr10: 8076002-8077261 |
| 770 | chr13: 112717125-112717421 |
| 771 | chr15: 89914363-89915061 |
| 772 | chr1: 228785986-228786204 |
| 773 | chr1: 156358050-156358252 |
| 774 | chr7: 751712-752150 |
| 775 | chr3: 137489051-137489409 |
| 776 | chr17: 7905927-7907445 |
| 777 | chr18: 35144907-35147628 |
| 778 | chr3: 9177691-9178189 |
| 779 | chr6: 10390888-10391098 |
| 780 | chr14: 37052537-37052838 |
| 781 | chr1: 47909712-47911020 |
| 782 | chr13: 93879245-93880877 |
| 783 | chr1: 50893468-50893745 |
| 784 | chr7: 27282086-27283136 |
| 785 | chr4: 147558231-147558583 |
| 786 | chr19: 13124569-13124788 |
| 787 | chr17: 46619087-46619314 |
| 788 | chr3: 44596535-44597018 |
| 789 | chr14: 24803678-24804353 |
| 790 | chr2: 3286324-3286530 |
| 791 | chr12: 14134626-14135242 |
| 792 | chr12: 114881649-114881937 |
| 793 | chr20: 22548967-22549720 |
| 794 | chr8: 37822486-37824008 |
| 795 | chr13: 100641334-100642188 |
| 796 | chr4: 206377-206892 |
| 797 | chr3: 11034446-11035384 |
| 798 | chr7: 152622343-152623305 |
| 799 | chr10: 22629360-22630328 |
| 800 | chr4: 140201064-140201449 |
| 801 | chr19: 46318490-46319266 |
| 802 | chr3: 121902742-121903645 |
| 803 | chr9: 77112712-77113583 |
| 804 | chr2: 114256775-114258043 |
| 805 | chr10: 15761423-15762101 |
| 806 | chr1: 115880167-115881332 |
| 807 | chr6: 50791110-50791573 |
| 808 | chr6: 55039170-55039392 |
| 809 | chr2: 176980765-176981423 |
| 810 | chr8: 86350765-86351196 |
| 811 | chr8: 24812946-24814299 |
| 812 | chr7: 19184818-19185033 |
| 813 | chr5: 76936126-76936984 |
| 814 | chr5: 87980878-87981272 |
| 815 | chr9: 77111778-77112042 |
| 816 | chr11: 20622720-20623399 |
| 817 | chr1: 50882433-50882660 |
| 818 | chr17: 35291899-35300875 |
| 819 | chr17: 46675044-46675589 |
| 820 | chr20: 5296266-5297798 |
| 821 | chr7: 156871054-156871297 |
| 822 | chr4: 681313-681514 |
| 823 | chr2: 177039551-177039951 |
| 824 | chr17: 46695325-46695553 |
| 825 | chr1: 41283840-41284591 |
| 826 | chr9: 16726859-16727273 |
| 827 | chr1: 65991001-65991811 |
| 828 | chr1: 181452706-181453073 |
| 829 | chr8: 120428398-120429178 |
| 830 | chr3: 32863174-32863415 |
| 831 | chr4: 134069162-134070442 |
| 832 | chr12: 123754049-123754373 |
| 833 | chr5: 63256548-63257886 |
| 834 | chr5: 1879689-1879928 |
| 835 | chr10: 118899247-118900329 |
| 836 | chr20: 2731063-2731395 |
| 837 | chr5: 134385967-134386370 |
| 838 | chr2: 177014948-177015214 |
| 839 | chr1: 67218079-67218293 |
| 840 | chr11: 65408344-65408631 |
| 841 | chr7: 156801418-156801632 |
| 842 | chr18: 54788959-54789194 |
| 843 | chr2: 220173870-220174283 |
| 844 | chr2: 220173021-220173271 |
| 845 | chr12: 113908887-113910681 |
| 846 | chr6: 100897080-100897621 |
| 847 | chr1: 155290606-155291001 |
| 848 | chr2: 130763483-130763764 |
| 849 | chr12: 129337870-129338653 |
| 850 | chr21: 34395128-34400245 |
| 851 | chr12: 52115410-52115679 |
| 852 | chr3: 126113547-126113967 |
| 853 | chr16: 3220438-3221356 |
| 854 | chr1: 119543056-119543454 |
| 855 | chr14: 62279476-62280019 |
| 856 | chr11: 636906-640628 |
| 857 | chr10: 102893660-102895059 |
| 858 | chr3: 3840513-3842772 |
| 859 | chr1: 119529819-119530712 |
| 860 | chr9: 32782936-32783625 |
| 861 | chr19: 1064897-1065191 |
| 862 | chr5: 54527319-54527760 |
| 863 | chr7: 156795355-156799394 |
| 864 | chr1: 155147185-155147444 |
| 865 | chr9: 37002489-37002957 |
| 866 | chr11: 69831571-69832484 |
| 867 | chr2: 128421719-128422182 |
| 868 | chr22: 38476836-38478839 |
| 869 | chr19: 54412710-54413087 |
| 870 | chr9: 123656750-123656972 |
| 871 | chr7: 129422997-129423355 |
| 872 | chr19: 36336275-36337138 |
| 873 | chr2: 50574045-50574817 |
| 874 | chr10: 102975969-102978096 |
| 875 | chr6: 5996185-5996486 |
| 876 | chr3: 26664104-26664796 |
| 877 | chr7: 155170623-155170939 |
| 878 | chr8: 65286067-65286659 |
| 879 | chr14: 37125219-37125661 |
| 880 | chr11: 65816404-65816665 |
| 881 | chr6: 41908745-41909711 |
| 882 | chr17: 46620367-46621373 |
| 883 | chr2: 142887724-142888553 |
| 884 | chr1: 221050448-221050864 |
| 885 | chr12: 106974412-106974951 |
| 886 | chr14: 57278068-57278287 |
| 887 | chr1: 67773329-67773767 |
| 888 | chr17: 40936445-40936668 |
| 889 | chr20: 2729997-2730797 |
| 890 | chr12: 113013099-113013529 |
| 891 | chr7: 155244046-155244357 |
| 892 | chr1: 214153214-214153668 |
| 893 | chr1: 156863415-156863711 |
| 894 | chr1: 114695136-114696672 |
| 895 | chr14: 85996494-85996958 |
| 896 | chr7: 100823307-100823701 |
| 897 | chr20: 52789252-52790986 |
| 898 | chr5: 178421225-178422337 |
| 899 | chr11: 36397926-36399398 |
| 900 | chr13: 36052553-36053119 |
| 901 | chr14: 57283967-57284558 |
| 902 | chr4: 25090106-25090510 |
| 903 | chr2: 5831187-5831413 |
| 904 | chr6: 117869097-117869530 |
| 905 | chr19: 58094739-58095764 |
| 906 | chr4: 85422929-85423190 |
| 907 | chr13: 100547172-100547431 |
| 908 | chr8: 68864584-68864946 |
| 909 | chr16: 49311413-49312308 |
| 910 | chr7: 19184221-19184686 |
| 911 | chr2: 19562749-19562965 |
| 912 | chr19: 54481412-54481955 |
| 913 | chr10: 124901907-124902617 |
| 914 | chr3: 62357639-62359774 |
| 915 | chr11: 31827696-31827921 |
| 916 | chr17: 43037166-43037740 |
| 917 | chr7: 37955622-37956555 |
| 918 | chr6: 106429111-106429772 |
| 919 | chr6: 50682334-50683214 |
| 920 | chr5: 76923887-76924502 |
| 921 | chr6: 168841818-168843100 |
| 922 | chr7: 19145872-19146256 |
| 923 | chr20: 32856659-32857248 |
| 924 | chr17: 79859808-79860963 |
| 925 | chr7: 95225503-95226194 |
| 926 | chr14: 105167663-105168129 |
| 927 | chr17: 14248391-14248721 |
| 928 | chr16: 84002269-84002860 |
| 929 | chr9: 104499849-104501076 |
| 930 | chr17: 46604362-46604881 |
| 931 | chr2: 87015974-87018182 |
| 932 | chr14: 36990873-36991209 |
| 933 | chr5: 52777788-52777996 |
| 934 | chr19: 35633847-35634629 |
| 935 | chr1: 221055492-221055800 |
| 936 | chr1: 146551476-146551764 |
| 937 | chr13: 100642774-100643094 |
| 938 | chr14: 85999532-86000478 |
| 939 | chr13: 36049570-36050159 |
| 940 | chr2: 119606038-119606313 |
| 941 | chr11: 123065426-123066184 |
| 942 | chr3: 172167526-172167866 |
| 943 | chr4: 41882450-41882964 |
| 944 | chr8: 142528185-142529029 |
| 945 | chr9: 79637814-79638169 |
| 946 | chr3: 19189688-19190100 |
| 947 | chr4: 122301567-122302290 |
| 948 | chr10: 130339526-130339777 |
| 949 | chr9: 35846310-35846638 |
| 950 | chr15: 53097561-53098476 |
| 951 | chr2: 157184389-157184632 |
| 952 | chr5: 145718289-145720095 |
| 953 | chr11: 105481126-105481422 |
| 954 | chr5: 170741603-170742751 |
| 955 | chr3: 62355315-62355534 |
| 956 | chr1: 38219702-38220012 |
| 957 | chr4: 41881177-41881418 |
| 958 | chr13: 112715359-112716234 |
| 959 | chr17: 1880789-1881116 |
| 960 | chr18: 56887091-56887665 |
| 961 | chr6: 10390038-10390565 |
| 962 | chr11: 69516931-69517218 |
| 963 | chr19: 39737689-39739288 |
| 964 | chr3: 157812053-157812764 |
| 965 | chr14: 37049333-37051726 |
| 966 | chr7: 156409023-156409294 |
| 967 | chr11: 46366876-46367101 |
| 968 | chr5: 50685453-50686148 |
| 969 | chr4: 41883492-41884570 |
| 970 | chr13: 112709884-112712665 |
| 971 | chr22: 44287497-44288061 |
| 972 | chr22: 46440393-46441019 |
| 973 | chr8: 23562475-23565175 |
| 974 | chr2: 207506774-207507422 |
| 975 | chr4: 169799086-169799625 |
| 976 | chr3: 133393118-133393657 |
| 977 | chr8: 41424341-41425300 |
| 978 | chr4: 100870377-100871994 |
| 979 | chr4: 107956555-107957453 |
| 980 | chr17: 79314962-79320653 |
| 981 | chr2: 30453566-30455655 |
| 982 | chr1: 18956895-18959829 |
| 983 | chr12: 41086522-41087102 |
| 984 | chr22: 42685894-42686095 |
| 985 | chr6: 100914946-100915245 |
| 986 | chr1: 46951168-46951792 |
| 987 | chr4: 41749184-41749811 |
| 988 | chr11: 128419198-128419513 |
| 989 | chr2: 171671598-171671804 |
| 990 | chr1: 170630456-170630851 |
| 991 | chr20: 44657463-44659243 |
| 992 | chr9: 139096665-139096993 |
| 993 | chr7: 155174128-155175248 |
| 994 | chr 14: 36993488-36994488 |
| 995 | chr3: 138654837-138655363 |
| 996 | chr4: 5709985-5710495 |
| 997 | chr15: 23157794-23158624 |
| 998 | chr20: 9496471-9496893 |
| 999 | chr4: 174437914-174438346 |
| 1000 | chr5: 140305712-140307193 |
| 1001 | chr15: 79576059-79576270 |
| 1002 | chr14: 38678245-38680937 |
| 1003 | chr10: 102473206-102474026 |
| 1004 | chr17: 59486727-59487132 |
| 1005 | chr3: 64253533-64253819 |
| 1006 | chr10: 102484200-102484476 |
| 1007 | chr7: 27198182-27198514 |
| 1008 | chr2: 97192977-97193383 |
| 1009 | chr9: 77113709-77113927 |
| 1010 | chr6: 154360586-154361008 |
| 1011 | chr11: 44324875-44325087 |
| 1012 | chr2: 182521221-182521927 |
| 1013 | chr7: 124404700-124406189 |
| 1014 | chr2: 132182327-132183101 |
| 1015 | chr7: 101005899-101007443 |
| 1016 | chr7: 149744402-149746469 |
| 1017 | chr8: 50822270-50822860 |
| 1018 | chr7: 27227520-27229043 |
| 1019 | chr6: 134212690-134213098 |
| 1020 | chr13: 36044844-36045481 |
| 1021 | chr11: 132934059-132934291 |
| 1022 | chr16: 51189800-51190260 |
| 1023 | chr1: 155145342-155145938 |
| 1024 | chr4: 682724-683079 |
| 1025 | chr5: 92939795-92940216 |
| 1026 | chr10: 134597357-134602649 |
| 1027 | chr1: 200009807-200010036 |
| 1028 | chr19: 12666243-12666682 |
| 1029 | chr9: 97401286-97402067 |
| 1030 | chr2: 107103833-107104053 |
| 1031 | chr15: 89910521-89912177 |
| 1032 | chr5: 140789094-140789762 |
| 1033 | chr2: 114033359-114033617 |
| 1034 | chr17: 12568667-12569335 |
| 1035 | chr11: 68622108-68622339 |
| 1036 | chr1: 160340604-160340843 |
| 1037 | chr7: 103085710-103086132 |
| 1038 | chr15: 76628998-76629207 |
| 1039 | chr20: 10198135-10198984 |
| 1040 | chr20: 44660342-44660948 |
| 1041 | chr17: 35290403-35290663 |
| 1042 | chr17: 933026-933236 |
| 1043 | chr4: 128544031-128544903 |
| 1044 | chr1: 50881884-50882103 |
| 1045 | chr10: 125425495-125426642 |
| 1046 | chr17: 46801784-46802071 |
| 1047 | chr1: 25255527-25259005 |
| 1048 | chr3: 32861141-32861429 |
| 1049 | chr17: 70116274-70119998 |
| 1050 | chr10: 75407413-75407706 |
| 1051 | chr2: 467849-468659 |
| 1052 | chr11: 132952538-132953307 |
| 1053 | chr3: 6904133-6904641 |
| 1054 | chr10: 120353692-120355821 |
| 1055 | chr7: 20830567-20830817 |
| 1056 | chr11: 71950815-71951408 |
| 1057 | chr14: 95240083-95240341 |
| 1058 | chr19: 5829048-5829474 |
| 1059 | chr20: 9495253-9495597 |
| 1060 | chr9: 112083333-112083549 |
| 1061 | chr15: 96873408-96877721 |
| 1062 | chr16: 67208067-67208678 |
| 1063 | chr1: 175568376-175568808 |
| 1064 | chr6: 5999149-5999787 |
| 1065 | chr3: 129693127-129694841 |
| 1066 | chr6: 10383525-10384114 |
| 1067 | chr11: 636435-636668 |
| 1068 | chr1: 181451311-181452049 |
| 1069 | chr9: 135464586-135466240 |
| 1070 | chr15: 60289325-60289533 |
| 1071 | chr16: 49309123-49309353 |
| 1072 | chr1: 243646394-243646888 |
| 1073 | chr12: 54071053-54071265 |
| 1074 | chr1: 91176404-91176701 |
| 1075 | chr5: 140864527-140864748 |
| 1076 | chr4: 47034427-47034940 |
| 1077 | chr10: 102489343-102491011 |
| 1078 | chr10: 102419147-102419668 |
| 1079 | chr12: 81471569-81472119 |
| 1080 | chr6: 50813314-50813699 |
| 1081 | chr5: 158526133-158526431 |
| 1082 | chr1: 119543821-119544339 |
| 1083 | chr5: 77140542-77140914 |
| 1084 | chr8: 23567180-23567678 |
| 1085 | chr1: 41831976-41832542 |
| 1086 | chr2: 139537692-139538650 |
| 1087 | chr7: 100075303-100075551 |
| 1088 | chr2: 176969217-176969895 |
| 1089 | chr7: 27284639-27286237 |
| 1090 | chr5: 31193952-31194419 |
| 1091 | chr6: 37616393-37616621 |
| 1092 | chr19: 1748167-1750243 |
| 1093 | chr10: 101281181-101282116 |
| 1094 | chr21: 31311386-31312106 |
| 1095 | chr2: 176973427-176973718 |
| 1096 | chr15: 96900142-96900644 |
| 1097 | chr7: 158936507-158938492 |
| 1098 | chr3: 63263989-63264205 |
| 1099 | chr16: 71459781-71460338 |
| 1100 | chr7: 155601175-155603235 |
| 1101 | chr12: 54447744-54448091 |
| 1102 | chr12: 53491572-53491955 |
| 1103 | chr10: 16561604-16563822 |
| 1104 | chr11: 133994709-133995090 |
| 1105 | chr2: 137522460-137523696 |
| 1106 | chr17: 12877270-12877773 |
| 1107 | chr8: 98289604-98290404 |
| 1108 | chr4: 185937242-185937750 |
| 1109 | chr3: 185911344-185912228 |
| 1110 | chr12: 54378696-54380102 |
| 1111 | chr1: 221060850-221061071 |
| 1112 | chr12: 63543636-63544967 |
| 1113 | chr6: 6006689-6007043 |
| 1114 | chr19: 51169659-51172023 |
| 1115 | chr1: 1474962-1475220 |
| 1116 | chr14: 54418677-54418881 |
| 1117 | chr6: 108497595-108497996 |
| 1118 | chr17: 37764092-37764304 |
| 1119 | chr4: 109092578-109092839 |
| 1120 | chr1: 91182097-91182364 |
| 1121 | chr13: 112760865-112761113 |
| 1122 | chr12: 122018170-122018457 |
| 1123 | chr7: 142494563-142495248 |
| 1124 | chr13: 58203586-58204322 |
| 1125 | chr1: 92945907-92952609 |
| 1126 | chr12: 106977388-106977713 |
| 1127 | chr5: 76925445-76926875 |
| 1128 | chr16: 3190765-3191389 |
| 1129 | chr1: 12123488-12124148 |
| 1130 | chr17: 48545570-48546900 |
| 1131 | chr12: 113916433-113916717 |
| 1132 | chr4: 41747508-41747944 |
| 1133 | chr19: 46916587-46916862 |
| 1134 | chr15: 49254984-49255564 |
| 1135 | chr19: 8674332-8674764 |
| 1136 | chr2: 223167205-223167560 |
| 1137 | chr17: 1173535-1174733 |
| 1138 | chr3: 75955759-75956308 |
| 1139 | chr5: 115697134-115697589 |
| 1140 | chr8: 21644908-21647845 |
| 1141 | chr5: 59189046-59189894 |
| 1142 | chr12: 54338761-54339168 |
| 1143 | chr16: 31053479-31053800 |
| 1144 | chr1: 50892437-50893243 |
| 1145 | chr17: 40935964-40936180 |
| 1146 | chr19: 44203558-44203987 |
| 1147 | chr4: 81109887-81110460 |
| 1148 | chr1: 2979275-2980758 |
| 1149 | chr16: 49872449-49872926 |
| 1150 | chr1: 200008392-200009047 |
| 1151 | chr16: 49316997-49317263 |
| 1152 | chr2: 114034594-114036041 |
| 1153 | chr2: 105480197-105480760 |
| 1154 | chr18: 44777632-44778084 |
| 1155 | chr19: 13213450-13213821 |
| 1156 | chr17: 6616422-6617471 |
| 1157 | chr14: 36977518-36977996 |
| 1158 | chr1: 214160798-214161034 |
| 1159 | chr1: 91182509-91182857 |
| 1160 | chr10: 130508443-130508658 |
| 1161 | chr2: 154728944-154729328 |
| 1162 | chr15: 89952271-89953061 |
| 1163 | chr18: 55102427-55102708 |
| 1164 | chr22: 31198491-31199033 |
| 1165 | chr10: 50821487-50821688 |
| 1166 | chr7: 100076454-100076785 |
| 1167 | chr18: 13641584-13642415 |
| 1168 | chr18: 13868532-13869026 |
| 1169 | chr6: 168841438-168841699 |
| 1170 | chr1: 61515875-61516831 |
| 1171 | chr7: 32110063-32110910 |
| 1172 | chr7: 56355508-56355798 |
| 1173 | chr19: 12767749-12767980 |
| 1174 | chr19: 19371675-19372393 |
| 1175 | chr14: 69256676-69257036 |
| 1176 | chr17: 75447477-75447821 |
| 1177 | chr14: 24801680-24802153 |
| 1178 | chr5: 148033472-148034080 |
| 1179 | chr10: 125650820-125651373 |
| 1180 | chr11: 43568921-43569854 |
| 1181 | chr22: 37212769-37213467 |
| 1182 | chr2: 162283581-162284677 |
| 1183 | chr8: 130995921-130996149 |
| 1184 | chr11: 70508328-70508617 |
| 1185 | chr16: 88943427-88943669 |
| 1186 | chr19: 42891311-42891646 |
| 1187 | chr15: 53079220-53079579 |
| 1188 | chr17: 46690390-46691055 |
| 1189 | chr4: 41880224-41880500 |
| 1190 | chr1: 156105707-156106171 |
| 1191 | chr6: 5997027-5997414 |
| 1192 | chr1: 18964180-18964401 |
| 1193 | chr14: 36983440-36983738 |
| 1194 | chr12: 54445876-54446113 |
| 1195 | chr5: 87968635-87968907 |
| 1196 | chr1: 29587087-29587412 |
| 1197 | chr11: 60718428-60718888 |
| 1198 | chr2: 66672431-66673636 |
| 1199 | chr4: 81119095-81119391 |
| 1200 | chr10: 76573195-76573507 |
| 1201 | chr22: 42322043-42322909 |
| 1202 | chr19: 45898879-45900315 |
| 1203 | chr14: 95826675-95826941 |
| 1204 | chr17: 48194634-48195085 |
| 1205 | chr19: 49669275-49669552 |
| 1206 | chr15: 96897596-96898046 |
| 1207 | chr19: 40314926-40315144 |
| 1208 | chr9: 120507227-120507642 |
| 1209 | chr5: 145722467-145722925 |
| 1210 | chr3: 19188246-19188772 |
| 1211 | chr5: 140787447-140788044 |
| 1212 | chr19: 50881418-50881664 |
| 1213 | chr10: 102896342-102896665 |
| 1214 | chr7: 53286851-53287192 |
| 1215 | chr15: 89903446-89903720 |
| 1216 | chr10: 23461300-23461610 |
| 1217 | chr2: 127783081-127783311 |
| 1218 | chr11: 72532612-72533774 |
| 1219 | chr2: 119605200-119605620 |
| 1220 | chr18: 12254147-12255089 |
| 1221 | chr7: 100817759-100817975 |
| 1222 | chr14: 77736733-77737772 |
| 1223 | chr12: 127212279-127212529 |
| 1224 | chr2: 119606569-119606826 |
| 1225 | chr1: 155264318-155265536 |
| 1226 | chr12: 131199824-131200157 |
| 1227 | chr1: 91300979-91301891 |
| 1228 | chr6: 100909210-100909444 |
| 1229 | chr6: 4079052-4079443 |
| 1230 | chr2: 233251361-233253414 |
| 1231 | chr4: 960505-960836 |
| 1232 | chr19: 21769189-21769786 |
| 1233 | chr10: 102279162-102279730 |
| 1234 | chr12: 127210778-127211651 |
| 1235 | chr12: 54069625-54070177 |
| 1236 | chr15: 53087211-53087488 |
| 1237 | chr13: 28365545-28365785 |
| 1238 | chr12: 113913615-113914322 |
| 1239 | chr14: 51338712-51339146 |
| 1240 | chr7: 155604725-155605095 |
| 1241 | chr3: 62364017-62364316 |
| 1242 | chr6: 6008857-6009299 |
| 1243 | chr3: 46618307-46618669 |
| 1244 | chr17: 33776553-33776888 |
| 1245 | chr12: 58158855-58160000 |
| 1246 | chr2: 219857682-219858917 |
| 1247 | chr19: 44278273-44278777 |
| 1248 | chr10: 101282725-101282934 |
| 1249 | chr20: 2539133-2539877 |
| 1250 | chr12: 58003880-58004249 |
| 1251 | chr16: 51147490-51147944 |
| 1252 | chr1: 179544720-179545307 |
| 1253 | chr2: 71787430-71787897 |
| 1254 | chr10: 129534410-129537366 |
| 1255 | chr6: 42145847-42146053 |
| 1256 | chr14: 24802927-24803159 |
| 1257 | chr22: 29707479-29707797 |
| 1258 | chr9: 132459587-132460017 |
| 1259 | chr 17: 40937258-40937480 |
| 1260 | chr4: 151504011-151505085 |
| 1261 | chr1: 18967251-18968119 |
| 1262 | chr19: 56598038-56600296 |
| 1263 | chr19: 35633409-35633697 |
| 1264 | chr2: 171678546-171680358 |
| 1265 | chr6: 134638797-134639021 |
| 1266 | chr1: 36549554-36549965 |
| 1267 | chr19: 12833104-12833574 |
| 1268 | chr3: 137487429-137488021 |
| 1269 | chr9: 139715663-139716441 |
| 1270 | chr6: 37617863-37618147 |
| 1271 | chr17: 32484007-32484280 |
| 1272 | chr7: 156409577-156409865 |
| 1273 | chr5: 11384681-11385521 |
| 1274 | chr8: 102504478-102504841 |
| 1275 | chr20: 33296514-33298242 |
| 1276 | chr20: 57415135-57417153 |
| 1277 | chr10: 71331449-71331691 |
| 1278 | chr3: 75667777-75669067 |
| 1279 | chr16: 67571252-67572728 |
| 1280 | chr19: 36500169-36500530 |
| 1281 | chr2: 154729613-154729918 |
| 1282 | chr12: 48399168-48399372 |
| 1283 | chr4: 41867385-41867586 |
| 1284 | chr17: 46800533-46800746 |
| 1285 | chr20: 44685771-44687610 |
| 1286 | chr19: 10406934-10407342 |
| 1287 | chr6: 108496715-108497320 |
| 1288 | chr5: 158523906-158524598 |
| 1289 | chr9: 124413512-124414193 |
| 1290 | chr20: 57427691-57427995 |
| 1291 | chr16: 10912159-10912719 |
| 1292 | chr7: 149389654-149389976 |
| 1293 | chr1: 173638662-173639045 |
| 1294 | chr19: 55597977-55598887 |
| 1295 | chr14: 62279037-62279339 |
| 1296 | chr3: 13114627-13115245 |
| 1297 | chr2: 3750828-3751927 |
| 1298 | chr4: 85402764-85403175 |
| 1299 | chr17: 74017769-74018658 |
| 1300 | chr5: 54523676-54523901 |
| 1301 | chr7: 89747892-89749036 |
| 1302 | chr18: 72916107-72917233 |
| 1303 | chr9: 136294738-136295236 |
| 1304 | chr1: 201252452-201253648 |
| 1305 | chr5: 146888750-146889840 |
| 1306 | chr14: 52734207-52735486 |
| 1307 | chr13: 20875518-20876214 |
| 1308 | chr18: 77560088-77560292 |
| 1309 | chr2: 102803672-102804556 |
| 1310 | chr2: 176982107-176982402 |
| 1311 | chr17: 6679205-6679710 |
| 1312 | chr19: 10463626-10464378 |
| 1313 | chr5: 140810494-140812617 |
| 1314 | chr11: 46299544-46300216 |
| 1315 | chr11: 64136814-64138187 |
| 1316 | chr6: 6007387-6007797 |
| 1317 | chr17: 37321482-37322099 |
| 1318 | chr10: 94455524-94455896 |
| 1319 | chr13: 51417371-51418149 |
| 1320 | chr8: 11565217-11567212 |
| 1321 | chr1: 226127112-226127695 |
| 1322 | chr2: 3287874-3288228 |
| 1323 | chr6: 10882926-10883149 |
| 1324 | chr22: 19746155-19746369 |
| 1325 | chr3: 12838471-12838782 |
| 1326 | chr9: 36739534-36739782 |
| 1327 | chr9: 134429866-134430491 |
| 1328 | chr11: 70672834-70673055 |
| 1329 | chr14: 24641053-24642220 |
| 1330 | chr7: 27283408-27283614 |
| 1331 | chr12: 49182421-49182658 |
| 1332 | chr1: 44031286-44031853 |
| 1333 | chr1: 114696886-114697185 |
| 1334 | chr15: 89901914-89902785 |
| 1335 | chr11: 65352231-65353134 |
| 1336 | chr7: 72838383-72838815 |
| 1337 | chr22: 38379093-38379964 |
| 1338 | chr4: 155663809-155664315 |
| 1339 | chr9: 100619984-100620192 |
| 1340 | chr7: 143582125-143582610 |
| 1341 | chr7: 23287221-23287508 |
| 1342 | chr11: 64815040-64815722 |
| 1343 | chr2: 87088816-87089037 |
| 1344 | chr20: 57426729-57427047 |
| 1345 | chr10: 43428167-43429460 |
| 1346 | chr10: 121577529-121578385 |
| 1347 | chr4: 190939801-190940591 |
| 1348 | chr6: 100037323-100037544 |
| 1349 | chr19: 12880574-12880888 |
| 1350 | chr2: 171670110-171670549 |
| 1351 | chr7: 124404174-124404432 |
| 1352 | chr7: 97840559-97840845 |
| 1353 | chr19: 50879606-50880094 |
| 1354 | chr1: 113265573-113265787 |
| 1355 | chr19: 2424005-2427983 |
| 1356 | chr3: 127633993-127634588 |
| 1357 | chr10: 50817095-50817309 |
| 1358 | chr2: 171676552-171676980 |
| 1359 | chr1: 86621278-86622871 |
| 1360 | chr1: 164545540-164545917 |
| 1361 | chr22: 19967279-19967808 |
| 1362 | chr11: 67350928-67351953 |
| 1363 | chr20: 36226617-36226841 |
| 1364 | chr19: 14089570-14089796 |
| 1365 | chr19: 38700333-38700577 |
| 1366 | chr1: 18435566-18435904 |
| 1367 | chr8: 21905461-21905757 |
| 1368 | chr2: 176950595-176950846 |
| 1369 | chr17: 75251958-75252180 |
| 1370 | chr15: 37390175-37390380 |
| 1371 | chr9: 98113447-98113662 |
| 1372 | chr1: 40235767-40237190 |
| 1373 | chr8: 144811237-144811446 |
| 1374 | chr8: 99984584-99985072 |
| 1375 | chr7: 152621916-152622149 |
| 1376 | chr1: 40769186-40769871 |
| 1377 | chr19: 2428349-2428731 |
| 1378 | chr17: 15820620-15821325 |
| 1379 | chr22: 25081850-25082112 |
| 1380 | chr1: 19203874-19204234 |
| 1381 | chr20: 61703526-61704022 |
| 1382 | chr2: 237080188-237080432 |
| 1383 | chr1: 156338758-156339251 |
| 1384 | chr1: 149332993-149333389 |
| 1385 | chr22: 50496441-50497393 |
| 1386 | chr7: 27146069-27146600 |
| 1387 | chr13: 100547633-100548911 |
| 1388 | chr4: 190939007-190939274 |
| 1389 | chr7: 73894815-73895110 |
| 1390 | chr19: 35632356-35632572 |
| 1391 | chr16: 67918679-67918909 |
| 1392 | chr2: 108602824-108603467 |
| 1393 | chr2: 238864315-238865170 |
| 1394 | chr8: 144808221-144810978 |
| 1395 | chr8: 145101631-145101834 |
| 1396 | chr12: 132905449-132906206 |
| 1397 | chr6: 99275763-99276038 |
| 1398 | chr5: 140800760-140801072 |
| 1399 | chr17: 75242871-75243613 |
| 1400 | chr17: 41278134-41278460 |
| 1401 | chr12: 122016170-122017693 |
| 1402 | chr10: 131264948-131265710 |
| 1403 | chr17: 46631800-46632212 |
| 1404 | chr14: 105167277-105167501 |
| 1405 | chr10: 23982382-23982589 |
| 1406 | chr19: 50931270-50931638 |
| 1407 | chr3: 27771638-27771942 |
| 1408 | chr18: 74799144-74800038 |
| 1409 | chr1: 21616380-21617101 |
| 1410 | chr1: 147782066-147782473 |
| 1411 | chr7: 6590563-6590957 |
| 1412 | chr7: 97839862-97840222 |
| 1413 | chr12: 113914440-113914657 |
| 1414 | chr19: 7933263-7934898 |
| 1415 | chr20: 22559553-22560001 |
| 1416 | chr15: 53086629-53086858 |
| 1417 | chr10: 94180315-94180754 |
| 1418 | chr5: 140052059-140053381 |
| 1419 | chr10: 101287162-101287920 |
| 1420 | chr14: 38677154-38677787 |
| 1421 | chr22: 39262338-39263211 |
| 1422 | chr18: 74153239-74155073 |
| 1423 | chr15: 59157045-59157594 |
| 1424 | chr4: 963804-964115 |
| 1425 | chr11: 624780-625053 |
| 1426 | chr7: 1362811-1363643 |
| 1427 | chr19: 36246328-36247982 |
| 1428 | chr5: 54528095-54528404 |
| 1429 | chr12: 54359658-54359906 |
| 1430 | chr2: 127782613-127782829 |
| 1431 | chr19: 406131-406611 |
| 1432 | chr17: 46697413-46697701 |
| 1433 | chr18: 43608140-43608510 |
| 1434 | chr16: 23724270-23724775 |
| 1435 | chr18: 55922987-55924068 |
| 1436 | chr15: 60291879-60292167 |
| 1437 | chr14: 92788913-92789204 |
| 1438 | chr19: 1108394-1109610 |
| 1439 | chr11: 124628367-124629590 |
| 1440 | chr1: 32052471-32052771 |
| 1441 | chr19: 11594372-11594987 |
| 1442 | chr19: 870774-871318 |
| 1443 | chr2: 54086776-54087266 |
| 1444 | chr2: 241459632-241460047 |
| 1445 | chr7: 127990926-127992616 |
| 1446 | chr1: 208132327-208133117 |
| 1447 | chr7: 90893567-90896683 |
| 1448 | chr1: 41284847-41285149 |
| 1449 | chr11: 32452144-32452708 |
| 1450 | chr5: 77146998-77147785 |
| 1451 | chr19: 45901452-45901688 |
| 1452 | chr7: 6661875-6662695 |
| 1453 | chr6: 161188084-161188639 |
| 1454 | chr17: 934417-935088 |
| 1455 | chr11: 65409636-65410127 |
| 1456 | chr17: 19883325-19883610 |
| 1457 | chr18: 77549524-77550299 |
| 1458 | chr1: 38461584-38461988 |
| 1459 | chr19: 10464666-10464927 |
| 1460 | chr17: 70120139-70120442 |
| 1461 | chr7: 27147589-27148389 |
| 1462 | chr2: 31806545-31806782 |
| 1463 | chr11: 119292689-119292891 |
| 1464 | chr19: 18979351-18981200 |
| 1465 | chr6: 42879279-42879623 |
| 1466 | chr12: 130908777-130909191 |
| 1467 | chr17: 46629553-46629816 |
| 1468 | chr1: 202162958-202163390 |
| 1469 | chr17: 21367114-21367592 |
| 1470 | chr16: 84001805-84002011 |
| 1471 | chr1: 221057463-221057757 |
| 1472 | chr17: 27899511-27900067 |
| 1473 | chr15: 40268581-40269061 |
| 1474 | chr22: 37465056-37465331 |
| 1475 | chr17: 77805866-77809046 |
| 1476 | chr19: 13198699-13198999 |
| 1477 | chr3: 184056419-184056671 |
| 1478 | chr22: 37911979-37912258 |
| 1479 | chr19: 19368708-19369681 |
| 1480 | chr11: 64135815-64136381 |
| 1481 | chr18: 77552401-77552603 |
| 1482 | chr19: 58554354-58554587 |
| 1483 | chr20: 57414595-57414896 |
| 1484 | chr4: 190938106-190938848 |
| 1485 | chr5: 172110282-172111166 |
| 1486 | chr16: 68480864-68482822 |
| 1487 | chr9: 139395020-139395287 |
| 1488 | chr12: 113515164-113515970 |
| 1489 | chr1: 221054554-221054888 |
| 1490 | chr8: 144990270-145002135 |
| 1491 | chr9: 131154346-131155923 |
| 1492 | chr6: 150335525-150336278 |
| 1493 | chr9: 115824684-115825033 |
| 1494 | chr12: 54519768-54520457 |
| 1495 | chr6: 35479872-35480154 |
| 1496 | chr19: 3870788-3871043 |
| 1497 | chr19: 48965002-48965792 |
| 1498 | chr6: 35479388-35479678 |
| 1499 | chr12: 52408381-52408675 |
| 1500 | chr1: 221068782-221069159 |
| 1501 | chr6: 46655262-46656738 |
| 1502 | chr3: 55508336-55508708 |
| 1503 | chr1: 39980365-39981768 |
| 1504 | chr16: 3067521-3068358 |
| 1505 | chr1: 1473107-1473342 |
| 1506 | chr10: 105362549-105362827 |
| 1507 | chr17: 46698880-46699083 |
| 1508 | chr2: 198029068-198029438 |
| 1509 | chr20: 17209418-17209622 |
| 1510 | chr12: 49183049-49183282 |
| 1511 | chr16: 58030214-58031633 |
| 1512 | chr10: 94820026-94823252 |
| 1513 | chr11: 725596-726870 |
| 1514 | chr6: 170732119-170732442 |
| 1515 | chr12: 120835586-120835927 |
| 1516 | chr20: 36012595-36013439 |
| 1517 | chr8: 143545445-143546178 |
| 1518 | chr6: 27228100-27228364 |
| 1519 | chr21: 32624144-32624382 |
| 1520 | chr9: 95477296-95477708 |
| 1521 | chr10: 105420685-105421076 |
| 1522 | chr1: 1470604-1471450 |
| 1523 | chr1: 146552328-146552577 |
| 1524 | chr19: 33625467-33625805 |
| 1525 | chr11: 64478843-64479598 |
| 1526 | chr20: 57428308-57428516 |
| 1527 | chr7: 27182613-27185562 |
| 1528 | chr19: 51815157-51815458 |
| 1529 | chr17: 46607804-46608390 |
| 1530 | chr12: 52408860-52409121 |
| 1531 | chr19: 10405924-10406398 |
| 1532 | chr11: 14993452-14993661 |
| 1533 | chr19: 13135317-13136169 |
| 1534 | chr7: 750788-751237 |
| 1535 | chr1: 53742297-53742845 |
| 1536 | chr1: 200010625-200010832 |
| 1537 | chr5: 139138875-139139242 |
| 1538 | chr17: 45949676-45949885 |
| 1539 | chr3: 128722283-128723036 |
| 1540 | chr15: 89312719-89313183 |
| 1541 | chr9: 135039673-135039978 |
| 1542 | chr19: 12831793-12832225 |
| 1543 | chr20: 51589707-51590020 |
| 1544 | chr20: 3145121-3145746 |
| 1545 | chr8: 65710990-65711722 |
| 1546 | chr11: 128694084-128694688 |
| 1547 | chr2: 20870006-20871280 |
| 1548 | chr19: 18977466-18977833 |
| 1549 | chr3: 49947621-49948430 |
| 1550 | chr6: 30139718-30140263 |
| 1551 | chr12: 104697348-104697984 |
| 1552 | chr10: 105361784-105362188 |
| 1553 | chr6: 29894140-29895117 |
| 1554 | chr4: 187219320-187219745 |
| 1555 | chr15: 67073306-67073943 |
| 1556 | chr2: 220412341-220412678 |
| 1557 | chr6: 170730395-170730887 |
| 1558 | chr9: 115822071-115823416 |
| 1559 | chr1: 10764449-10764925 |
| 1560 | chr17: 46627787-46628444 |
| 1561 | chr19: 51601822-51602260 |
| 1562 | chr19: 55814067-55814278 |
| 1563 | chr6: 138745348-138745593 |
| 1564 | chr9: 124987743-124991086 |
| 1565 | chr22: 46318693-46319087 |
| 1566 | chr16: 3013016-3013228 |
| 1567 | chr4: 114900355-114900810 |
| 1568 | chr19: 1063544-1064265 |
| 1569 | chr19: 1110399-1110701 |
| 1570 | chr7: 97841636-97842005 |
| 1571 | chr8: 57359899-57360114 |
| 1572 | chr17: 72915568-72916510 |
| 1573 | chr1: 16860873-16862296 |
| 1574 | chr17: 75398284-75398527 |
| 1575 | chr9: 139397412-139397710 |
| 1576 | chr6: 33393592-33393908 |
| 1577 | chr6: 29595298-29595795 |
| 1578 | chr12: 6438272-6438931 |
| 1579 | chr3: 113160299-113160641 |
| 1580 | chr1: 55505060-55506015 |
| 1581 | chr11: 132951692-132952260 |
| 1582 | chr4: 81118137-81118603 |
| 1583 | chr19: 38876070-38876332 |
| 1584 | chr19: 58549305-58549712 |
| 1585 | chr17: 43472527-43474343 |
| 1586 | chr9: 139396205-139397040 |
| 1587 | chr16: 3192181-3192669 |
| 1588 | chr6: 33048416-33048814 |
| 1589 | chr7: 128555329-128556650 |
| 1590 | chr19: 46915311-46915802 |
| 1591 | chr6: 30095173-30095610 |

TABLE 2
Example CGIs
Human CGI (hg19)

| | |
|---|---|
| chr1: 1181756-1182470 | chr12: 103696090-103696418 |
| chr1: 1470604-1471450 | chr12: 104697348-104697984 |
| chr1: 2772126-2772665 | chr12: 106974412-106974951 |
| chr1: 4713989-4716555 | chr12: 113013099-113013529 |
| chr1: 18436551-18437673 | chr12: 113515164-113515970 |
| chr1: 18956895-18959829 | chr12: 113916433-113916717 |
| chr1: 18962842-18963481 | chr12: 114833911-114834210 |
| chr1: 18967251-18968119 | chr12: 114838312-114838889 |
| chr1: 19203874-19204234 | chr12: 114843022-114843610 |
| chr1: 21616380-21617101 | chr12: 114845861-114847650 |
| chr1: 25255527-25259005 | chr12: 114851957-114852360 |
| chr1: 29585897-29586598 | chr12: 114881649-114881937 |
| chr1: 34628783-34630976 | chr12: 114885105-114885418 |
| chr1: 39980365-39981768 | chr12: 119212110-119212393 |
| chr1: 40235767-40237190 | chr12: 123754049-123754373 |
| chr1: 41831976-41832542 | chr12: 127210778-127211651 |
| chr1: 46951168-46951792 | chr12: 127940451-127940907 |
| chr1: 47909712-47911020 | chr12: 129337870-129338653 |
| chr1: 53742297-53742845 | chr12: 131199824-131200157 |
| chr1: 55505060-55506015 | chr12: 132905449-132906206 |
| chr1: 61515875-61516831 | chr13: 20875518-20876214 |
| chr1: 63782394-63790471 | chr13: 28366549-28368505 |
| chr1: 65731411-65731849 | chr13: 28549839-28550246 |
| chr1: 66258440-66258918 | chr13: 36044844-36045481 |
| chr1: 77747314-77748224 | chr13: 51417371-51418149 |
| chr1: 91172102-91172771 | chr13: 53419897-53422872 |
| chr1: 91176404-91176701 | chr13: 58203586-58204322 |
| chr1: 92945907-92952609 | chr13: 58206526-58208930 |
| chr1: 115880167-115881332 | chr13: 79181944-79182222 |
| chr1: 116380359-116382364 | chr13: 93879245-93880877 |
| chr1: 156105707-156106171 | chr13: 100547633-100548911 |
| chr1: 156338758-156339251 | chr13: 100641334-100642188 |
| chr1: 156358050-156358252 | chr13: 102568425-102569495 |
| chr1: 156390403-156391581 | chr13: 112707804-112708696 |
| chr1: 160340604-160340843 | chr13: 112709884-112712665 |
| chr1: 161695637-161697298 | chr13: 112715359-112716234 |
| chr1: 177133392-177133846 | chr13: 112717125-112717421 |
| chr1: 180198119-180204975 | chr13: 112720564-112723582 |
| chr1: 197887088-197887791 | chr13: 112726281-112728419 |
| chr1: 201252452-201253648 | chr13: 112758598-112760491 |
| chr1: 202678881-202679769 | chr13: 112760865-112761113 |
| chr1: 214156000-214156851 | chr14: 24044886-24046760 |
| chr1: 214158726-214159080 | chr14: 24641053-24642220 |
| chr1: 221057463-221057757 | chr14: 24803678-24804353 |
| chr1: 221067447-221068185 | chr14: 29236835-29237832 |
| chr1: 226075150-226075680 | chr14: 29254365-29255069 |
| chr1: 248020330-248021252 | chr14: 33402094-33404079 |
| chr10: 50602989-50606783 | chr14: 36973169-36973740 |
| chr10: 50817601-50820356 | chr14: 36983440-36983738 |
| chr10: 71331926-71333392 | chr14: 36990873-36991209 |
| chr10: 88122924-88127364 | chr14: 36993488-36994488 |
| chr10: 94820026-94823252 | chr14: 37053134-37053690 |
| chr10: 101279941-101280382 | chr14: 37126786-37128274 |
| chr10: 101281181-101282116 | chr14: 37135513-37136348 |
| chr10: 102419147-102419668 | chr14: 38724254-38725537 |
| chr10: 102473206-102474026 | chr14: 48143433-48145589 |
| chr10: 102484200-102484476 | chr14: 51338712-51339146 |
| chr10: 102489343-102491011 | chr14: 52734207-52735486 |
| chr10: 102507482-102509646 | chr14: 57260878-57262123 |
| chr10: 102893660-102895059 | chr14: 57264638-57265561 |
| chr10: 102896342-102896665 | chr14: 57278709-57279116 |
| chr10: 102899822-102900263 | chr14: 58331676-58333121 |
| chr10: 102975969-102978096 | chr14: 60973772-60974123 |
| chr10: 105361784-105362188 | chr14: 60975732-60978180 |
| chr10: 105420685-105421076 | chr14: 61103978-61104663 |
| chr10: 106399567-106402812 | chr14: 62279476-62280019 |
| chr10: 118899247-118900329 | chr14: 77736733-77737772 |
| chr10: 119000435-119001530 | chr14: 85997468-85998637 |
| chr10: 119311204-119312104 | chr14: 85999532-86000478 |
| chr10: 119312766-119313563 | chr14: 92789494-92790712 |
| chr10: 124905634-124906161 | chr14: 95239375-95239679 |
| chr10: 124907283-124911035 | chr14: 95826675-95826941 |
| chr10: 129534410-129537366 | chr14: 101192851-101193499 |
| chr11: 725596-726870 | chr14: 101923575-101925995 |
| chr11: 8190226-8190671 | chr14: 103655241-103655928 |
| chr11: 17740789-17743779 | chr15: 23157794-23158624 |
| chr11: 20181200-20182325 | chr15: 27112030-27113479 |
| chr11: 20622720-20623399 | chr15: 27215951-27216856 |
| chr11: 31825743-31826967 | chr15: 33602816-33604003 |
| chr11: 31839363-31839813 | chr15: 35046443-35047480 |
| chr11: 31848487-31848776 | chr15: 37390175-37390380 |
| chr11: 32452144-32452708 | chr15: 53076187-53077926 |
| chr11: 32454874-32457311 | chr15: 53079220-53079579 |
| chr11: 36397926-36399398 | chr15: 53080458-53083699 |
| chr11: 44327240-44327932 | chr15: 53087211-53087488 |
| chr11: 46299544-46300216 | chr15: 53097561-53098476 |
| chr11: 46366876-46367101 | chr15: 59157045-59157594 |
| chr11: 64136814-64138187 | chr15: 76630029-76630970 |
| chr11: 65352231-65353134 | chr15: 79574830-79575211 |
| chr11: 69517840-69519929 | chr15: 89147660-89149198 |
| chr11: 69831571-69832484 | chr15: 89312719-89313183 |
| chr11: 70672834-70673055 | chr15: 89903446-89903720 |
| chr11: 72532612-72533774 | chr15: 89910521-89912177 |
| chr11: 79148358-79152200 | chr15: 89952271-89953061 |
| chr11: 124629723-124629926 | chr15: 96895306-96895729 |
| chr12: 3475010-3475654 | chr15: 96903311-96903711 |
| chr12: 5018585-5021171 | chr15: 96904722-96905050 |
| chr12: 6438272-6438931 | chr15: 96909815-96910030 |
| chr12: 15475318-15475901 | chr15: 96959341-96960531 |
| chr12: 29302034-29302954 | chr15: 100913438-100914022 |
| chr12: 45444202-45445386 | chr16: 3067521-3068358 |
| chr12: 49183049-49183282 | chr16: 3220438-3221356 |
| chr12: 49371690-49375550 | chr16: 6068914-6070401 |
| chr12: 49484920-49485178 | chr16: 10912159-10912719 |
| chr12: 53491572-53491955 | chr16: 20084707-20085305 |
| chr12: 54338761-54339168 | chr16: 23724270-23724775 |
| chr12: 54366815-54369103 | chr16: 24267040-24267527 |
| chr12: 54378696-54380102 | chr16: 31053479-31053800 |
| chr12: 54423427-54423712 | chr16: 49309123-49309353 |
| chr12: 54440642-54441543 | chr16: 49316997-49317263 |
| chr12: 54447744-54448091 | chr16: 51183699-51188763 |
| chr12: 54519768-54520457 | chr16: 54325040-54325703 |
| chr12: 57618769-57619402 | chr16: 55364823-55365483 |
| chr12: 58003880-58004249 | chr16: 66612749-66613412 |
| chr12: 58158855-58160000 | chr16: 67918679-67918909 |
| chr12: 63543636-63544967 | chr16: 71459781-71460338 |
| chr12: 75602991-75603344 | chr16: 82660651-82661813 |
| chr12: 99139386-99139769 | chr16: 84002269-84002860 |
| chr12: 101109863-101111622 | chr17: 934417-935088 |
| chr12: 106979429-106981086 | chr17: 1173535-1174733 |
| chr12: 113590806-113591304 | chr17: 1880789-1881116 |
| chr12: 113900750-113906442 | chr17: 5000369-5001205 |
| chr12: 113908887-113910681 | chr17: 6616422-6617471 |
| chr12: 113913615-113914322 | chr17: 6679205-6679710 |
| chr12: 114878143-114879155 | chr17: 7832532-7833164 |
| chr12: 114886354-114886579 | chr17: 7905927-7907445 |
| chr12: 115109503-115110061 | chr17: 12877270-12877773 |
| chr12: 117798076-117799448 | chr17: 14201726-14202052 |
| chr12: 120835586-120835927 | chr17: 15820620-15821325 |
| chr12: 122016170-122017693 | chr17: 19883325-19883610 |
| chr12: 130387609-130389139 | chr17: 21367114-21367592 |
| chr12: 130908777-130909191 | chr17: 27899511-27900067 |
| chr13: 27334226-27335205 | chr17: 33776553-33776888 |
| chr13: 28498226-28499046 | chr17: 36717727-36718593 |
| chr13: 36049570-36050159 | chr17: 37321482-37322099 |
| chr13: 36052553-36053119 | chr17: 43037166-43037740 |
| chr13: 79182859-79183880 | chr17: 46604362-46604881 |
| chr13: 84453664-84453897 | chr17: 46627787-46628444 |
| chr13: 108518334-108518633 | chr17: 46673532-46674181 |
| chr13: 109147798-109149019 | chr17: 46697413-46697701 |
| chr14: 36974548-36975425 | chr17: 46796234-46797292 |
| chr14: 36986362-36990576 | chr17: 46800533-46800746 |
| chr14: 37049333-37051726 | chr17: 46824785-46825372 |
| chr14: 37116188-37117628 | chr17: 48041282-48043064 |
| chr14: 38678245-38680937 | chr17: 48545570-48546900 |
| chr14: 54418677-54418881 | chr17: 59531723-59535254 |
| chr14: 57274607-57276840 | chr17: 70111979-70112308 |
| chr14: 57283967-57284558 | chr17: 70112824-70114271 |
| chr14: 69256676-69257036 | chr17: 71948478-71949255 |
| chr14: 74706188-74708192 | chr17: 73749618-73750178 |
| chr14: 95237622-95238211 | chr17: 74533281-74534566 |
| chr14: 105167663-105168129 | chr18: 904578-909574 |
| chr15: 33009530-33011696 | chr18: 11148307-11149936 |
| chr15: 40268581-40269061 | chr18: 11750953-11752756 |
| chr15: 45408573-45409528 | chr18: 12254147-12255089 |
| chr15: 47476369-47477499 | chr18: 13641584-13642415 |
| chr15: 49254984-49255564 | chr18: 13868532-13869026 |
| chr15: 60287107-60287663 | chr18: 43608140-43608510 |
| chr15: 60296135-60298520 | chr18: 44336183-44337110 |
| chr15: 67073306-67073943 | chr18: 44337510-44338100 |
| chr15: 74419870-74423044 | chr18: 44772992-44775577 |
| chr15: 79724099-79725643 | chr18: 44777632-44778084 |
| chr15: 89914363-89915061 | chr18: 44789742-44790678 |
| chr15: 89920793-89922768 | chr18: 54788959-54789194 |
| chr15: 89949373-89951130 | chr18: 55019707-55021605 |
| chr15: 91642908-91643702 | chr18: 55094825-55096310 |
| chr15: 96873408-96877721 | chr18: 56887091-56887665 |
| chr16: 2228190-2230946 | chr18: 56939624-56941540 |
| chr16: 3013016-3013228 | chr18: 70533965-70536871 |
| chr16: 3190765-3191389 | chr18: 72916107-72917233 |
| chr16: 22824616-22826459 | chr18: 73167402-73167920 |
| chr16: 48844551-48845264 | chr18: 74799144-74800038 |
| chr16: 49311413-49312308 | chr18: 76732970-76734765 |
| chr16: 49314037-49316543 | chr18: 76737005-76741244 |
| chr16: 49872449-49872926 | chr18: 77547965-77549038 |
| chr16: 51147490-51147944 | chr18: 77557780-77558948 |
| chr16: 51168266-51169110 | chr19: 870774-871318 |
| chr16: 54970301-54972846 | chr19: 3868586-3869217 |
| chr16: 55513220-55513526 | chr19: 5829048-5829474 |
| chr16: 58030214-58031633 | chr19: 8674332-8674764 |
| chr16: 62069121-62070634 | chr19: 10406934-10407342 |
| chr16: 67208067-67208678 | chr19: 10463626-10464378 |
| chr16: 67571252-67572728 | chr19: 12666243-12666682 |
| chr16: 68480864-68482822 | chr19: 12767749-12767980 |
| chr16: 86530747-86532994 | chr19: 12831793-12832225 |
| chr16: 86549069-86550512 | chr19: 12880574-12880888 |
| chr16: 86612188-86613821 | chr19: 13124959-13125259 |
| chr16: 88943427-88943669 | chr19: 13616752-13617267 |
| chr17: 12568667-12569335 | chr19: 14089570-14089796 |
| chr17: 14248391-14248721 | chr19: 19371675-19372393 |
| chr17: 32484007-32484280 | chr19: 21769189-21769786 |
| chr17: 35291899-35300875 | chr19: 33625467-33625805 |
| chr17: 37764092-37764304 | chr19: 36246328-36247982 |
| chr17: 40937258-40937480 | chr19: 36523391-36523887 |
| chr17: 43472527-43474343 | chr19: 38700333-38700577 |
| chr17: 45949676-45949885 | chr19: 39737689-39739288 |
| chr17: 46607804-46608390 | chr19: 39754973-39756540 |
| chr17: 46620367-46621373 | chr19: 40314926-40315144 |
| chr17: 46631800-46632212 | chr19: 44203558-44203987 |
| chr17: 46669434-46669811 | chr19: 44278273 -44278777 |
| chr17: 46691520-46692097 | chr19: 45260352-45261809 |
| chr17: 48194634-48195085 | chr19: 46001830-46002686 |
| chr17: 50235175-50236466 | chr19: 46318490-46319266 |
| chr17: 59485573-59485780 | chr19: 46915311-46915802 |
| chr17: 59528979-59530266 | chr19: 47151768-47153125 |
| chr17: 70116274-70119998 | chr19: 49669275-49669552 |
| chr17: 70120139-70120442 | chr19: 51601822-51602260 |
| chr17: 72855621-72858012 | chr19: 51815157-51815458 |
| chr17: 72915568-72916510 | chr19: 54412710-54413087 |
| chr17: 74017769-74018658 | chr19: 54481412-54481955 |
| chr17: 77805866-77809046 | chr19: 54483021-54483572 |
| chr17: 79314962-79320653 | chr19: 55597977-55598887 |
| chr17: 79859808-79860963 | chr19: 56988313-56989741 |
| chr18: 19744936-19752363 | chr19: 58094739-58095764 |
| chr18: 30349690-30352302 | chr19: 58545115-58545897 |
| chr18: 35144907-35147628 | chr19: 58554354-58554587 |
| chr18: 55103154-55108853 | chr2: 467849-468659 |
| chr18: 55922987-55924068 | chr2: 3286324-3286530 |
| chr18: 59000683-59001692 | chr2: 5831187-5831413 |
| chr18: 74153239-74155073 | chr2: 19560963-19561650 |
| chr18: 74961556-74963822 | chr2: 20870006-20871280 |
| chr19: 407011-409511 | chr2: 25499763-25500429 |
| chr19: 1063544-1064265 | chr2: 31805293-31806403 |
| chr19: 1 108394-1109610 | chr2: 45169505-45171884 |
| chr19: 1748167-1750243 | chr2: 45227644-45228783 |
| chr19: 2424005-2427983 | chr2: 45240372-45241579 |
| chr19: 7933263-7934898 | chr2: 54086776-54087266 |
| chr19: 1 1594372-11594987 | chr2: 63282514-63283122 |
| chr19: 13135317-13136169 | chr2: 63283936-63284147 |
| chr19: 13198699-13198999 | chr2: 63285949-63287097 |
| chr19: 13213450-13213821 | chr2: 66652691-66654218 |
| chr19: 18979351-18981200 | chr2: 66672431-66673636 |
| chr19: 19368708-19369681 | chr2: 80549578-80549798 |
| chr19: 30715549-30715753 | chr2: 87015974-87018182 |
| chr19: 35633409-35633697 | chr2: 87088816-87089037 |
| chr19: 36336275-36337138 | chr2: 97192977-97193383 |
| chr19: 36500169-36500530 | chr2: 105480197-105480760 |
| chr19: 38876070-38876332 | chr2: 106681982-106682403 |
| chr19: 42891311-42891646 | chr2: 107103833-107104053 |
| chr19: 45898879-45900315 | chr2: 114033359-114033617 |
| chr19: 48965002-48965792 | chr2: 114034594-114036041 |
| chr19: 50881418-50881664 | chr2: 114256775-114258043 |
| chr19: 50931270-50931638 | chr2: 118981769-118982466 |
| chr19: 51169659-51172023 | chr2: 119592602-119593845 |
| chr19: 55815940-55816277 | chr2: 119599059-119599299 |
| chr19: 56598038-56600296 | chr2: 119602616-119604486 |
| chr2: 3750828-3751927 | chr2: 119606569-119606826 |
| chr2: 30453566-30455655 | chr2: 119611296-119611881 |
| chr2: 38301276-38304518 | chr2: 119616133-119616826 |
| chr2: 45155195-45157049 | chr2: 119914126-119916663 |
| chr2: 45395869-45398186 | chr2: 124782252-124783255 |
| chr2: 50574045-50574817 | chr2: 127413696-127414171 |
| chr2: 66808568-66809404 | chr2: 127782613-127782829 |
| chr2: 71787430-71787897 | chr2: 128421719-128422182 |
| chr2: 73143055-73148260 | chr2: 130763483-130763764 |
| chr2: 80529677-80530846 | chr2: 132182327-132183101 |
| chr2: 102803672-102804556 | chr2: 139537692-139538650 |
| chr2: 105459127-105461770 | chr2: 154727906-154728271 |
| chr2: 105468851-105473488 | chr2: 154728944-154729328 |
| chr2: 108602824-108603467 | chr2: 162279835-162280709 |
| chr2: 119599458-119600966 | chr2: 162283581-162284677 |
| chr2: 137522460-137523696 | chr2: 171671598-171671804 |
| chr2: 142887724-142888553 | chr2: 171678546-171680358 |
| chr2: 144694666-144695180 | chr2: 176931575-176932663 |
| chr2: 157185557-157186355 | chr2: 176936246-176936809 |
| chr2: 162273294-162273725 | chr2: 176944087-176948446 |
| chr2: 176949511-176949795 | chr2: 176949993-176950336 |
| chr2: 176964062-176965509 | chr2: 176956504-176956707 |
| chr2: 176969217-176969895 | chr2: 177012371-177012675 |
| chr2: 176977284-176977540 | chr2: 177016416-177016632 |
| chr2: 176982107-176982402 | chr2: 177024501-177025692 |
| chr2: 177036254-177037213 | chr2: 198029068-198029438 |
| chr2: 177042751-177043444 | chr2: 200333687-200334172 |
| chr2: 182321761-182323029 | chr2: 207506774-207507422 |
| chr2: 182521221-182521927 | chr2: 220173 870-220174283 |
| chr2: 219736132-219736592 | chr2: 223159725-223160487 |
| chr2: 219848919-219850541 | chr2: 223162946-223163912 |
| chr2: 219857682-219858917 | chr2: 223167205-223167560 |
| chr2: 220299483-2203 00243 | chr2: 223168653-223169008 |
| chr2: 220412341 -220412678 | chr2: 223176493-223177515 |
| chr2: 223183013-223185468 | chr2: 233251361-233253414 |
| chr2: 237071794-237078762 | chr2: 237068071-237068834 |
| chr2: 241758141-241760783 | chr2: 238864315-238865170 |
| chr20: 3145121-3145746 | chr2: 241459632-241460047 |
| chr20: 21485932-21496714 | chr20: 690575-691099 |
| chr20: 21686199-21687689 | chr20: 2539133-2539877 |
| chr20: 22557517-22559240 | chr20: 2729997-2730797 |
| chr20: 33296514-33298242 | chr20: 2780978-2781497 |
| chr20: 37352130-37357372 | chr20: 5296266-5297798 |
| chr20: 39994545-39995810 | chr20: 9496471-9496893 |
| chr20: 44657463-44659243 | chr20: 10198135-10198984 |
| chr20: 44685771-44687610 | chr20: 17206528-17206952 |
| chr20: 51589707-51590020 | chr20: 17208550-17208756 |
| chr20: 52789252-52790986 | chr20: 21376358-21378245 |
| chr20: 57415135-57417153 | chr20: 21694472-21695344 |
| chr21: 31311386-31312106 | chr20: 22548967-22549720 |
| chr21: 32624144-32624382 | chr20: 25063838-25065525 |
| chr21: 3 8065179-38066185 | chr20: 32856659-32857248 |
| chr22: 19967279-19967808 | chr20: 36012595-36013439 |
| chr22: 29709281-29712013 | chr20: 36226617-36226841 |
| chr22: 31198491-31199033 | chr20: 41817475-41819212 |
| chr22: 31500396-31501239 | chr20: 48184193-48184833 |
| chr22: 37212769-37213467 | chr20: 57089460-57090237 |
| chr22: 37911979-37912258 | chr20: 57426729-57427047 |
| chr22: 38476836-38478839 | chr20: 61703526-61704022 |
| chr22: 42305617-42307254 | chr21: 19617098-19617874 |
| chr22: 42322043-42322909 | chr21: 34395128-34400245 |
| chr22: 44726724-44727590 | chr21: 3 8076762-38077685 |
| chr22: 46318693-46319087 | chr21: 38079941-38081833 |
| chr22: 46440393-46441019 | chr21: 42218489-42219222 |
| chr3: 3840513-3842772 | chr22: 19746155-19746369 |
| chr3: 6902823-6903516 | chr22: 25081850-25082112 |
| chr3: 13114627-13115245 | chr22: 37465056-37465331 |
| chr3: 19189688-19190100 | chr22: 38379093-38379964 |
| chr3: 49947621-49948430 | chr22: 39262338-39263211 |
| chr3: 55508336-55508708 | chr22: 42685894-42686095 |
| chr3: 62354291-62355012 | chr22: 44257942-44258612 |
| chr3: 62357639-62359774 | chr22: 44287497-44288061 |
| chr3: 71834068-71834653 | chr22: 48884884-48887043 |
| chr3: 87841796-87842563 | chr22: 50496441-50497393 |
| chr3: 137482964-137484454 | chr3: 238391-240140 |
| chr3: 137489594-137491004 | chr3: 6904133-6904641 |
| chr3: 147108511-147111703 | chr3: 9177691-9178189 |
| chr3: 147113608-147114479 | chr3: 11034446-11035384 |
| chr3: 147130342-147130577 | chr3: 12838471-12838782 |
| chr3: 147131066-147131333 | chr3: 22413492-22414365 |
| chr3: 154146347-154146965 | chr3: 26664104-26664796 |
| chr3: 157821232-157821604 | chr3: 27771638-27771942 |
| chr3: 170303044-170303249 | chr3: 32861141-32861429 |
| chr3: 172165372-172166738 | chr3: 44063314-44063837 |
| chr4: 4868440-4869173 | chr3: 44596535-44597018 |
| chr4: 25090106-25090510 | chr3: 46618307-46618669 |
| chr4: 41749184-41749811 | chr3: 62356119-62356378 |
| chr4: 47034427-47034940 | chr3: 62356773-62357315 |
| chr4: 54966163-54968063 | chr3: 62362610-62363082 |
| chr4: 81119095-81119391 | chr3: 63263989-63264205 |
| chr4: 90228714-90229010 | chr3: 64253533-64253819 |
| chr4: 94755786-94756310 | chr3: 75667777-75669067 |
| chr4: 100870377-100871994 | chr3: 75955759-75956308 |
| chr4: 107956555-107957453 | chr3: 113160299-113160641 |
| chr4: 109093038-109094546 | chr3: 121902742-121903645 |
| chr4: 114900355-114900810 | chr3: 126113547-126113967 |
| chr4: 1223 01567-122302290 | chr3: 127633993-127634588 |
| chr4: 128544031-128544903 | chr3: 127794369-127796136 |
| chr4: 144620822-144622218 | chr3: 128719865-128721245 |
| chr4: 147559205-147561901 | chr3: 129693127-129694841 |
| chr4: 156680095-156681386 | chr3: 133393118-133393657 |
| chr4: 164264821-164265772 | chr3: 138656627-138659107 |
| chr4: 172733734-172735118 | chr3: 147126988-147128999 |
| chr4: 174430386-174430861 | chr3: 147138916-147139564 |
| chr4: 185939222-185942747 | chr3: 147142181-147142391 |
| chr5: 1879689-1879928 | chr3: 157812053-157812764 |
| chr5: 1881924-1887743 | chr3: 170303532-170303768 |
| chr5: 2748368-2757024 | chr3: 184056419-184056671 |
| chr5: 37834671-37835128 | chr3: 185911344-185912228 |
| chr5: 38257825-38259136 | chr3: 186078710-186080111 |
| chr5: 52777788-52777996 | chr3: 192125821-192127994 |
| chr5: 54527319-54527760 | chr4: 107146-107898 |
| chr5: 59189046-59189894 | chr4: 206377-206892 |
| chr5: 63256548-63257886 | chr4: 682724-683079 |
| chr5: 71014917-71015715 | chr4: 961347-962155 |
| chr5: 72529099-72529976 | chr4: 4859632-4860191 |
| chr5: 76932317-76933523 | chr4: 5709985-5710495 |
| chr5: 76934581-76935296 | chr4: 5891981-5892365 |
| chr5: 77805753-77806313 | chr4: 5894071-5895116 |
| chr5: 92923487-92924497 | chr4: 13524062-13526083 |
| chr5: 92939795-92940216 | chr4: 15779998-15780729 |
| chr5: 134363092-134365146 | chr4: 24801109-24801902 |
| chr5: 134366913-134367438 | chr4: 41869174-41869459 |
| chr5: 134374385-134376751 | chr4: 41875445-41875794 |
| chr5: 139138875-139139242 | chr4: 41880224-41880500 |
| chr5: 140052059-140053381 | chr4: 41882450-41882964 |
| chr5: 140305712-140307193 | chr4: 46995128-46995872 |
| chr5: 140798757-140799359 | chr4: 54975387-54976202 |
| chr5: 140810494-140812617 | chr4: 57521621-57522703 |
| chr5: 145718289-145720095 | chr4: 66535193-66535620 |
| chr5: 145725286-145725852 | chr4: 81109887-81110460 |
| chr5: 158523906-158524598 | chr4: 85403830-85404524 |
| chr5: 172665306-172666072 | chr4: 85413997-85414874 |
| chr5: 179228283-179229003 | chr4: 85422929-85423190 |
| chr6: 391188-393790 | chr4: 93226348-93227007 |
| chr6: 1381743-1385211 | chr4: 110222970-110224257 |
| chr6: 5997027-5997414 | chr4: 111554965-111555504 |
| chr6: 6007387-6007797 | chr4: 134069162-134070442 |
| chr6: 7229877-7230865 | chr4: 140201064-140201449 |
| chr6: 10390038-10390565 | chr4: 151504011-151505085 |
| chr6: 29894140-29895117 | chr4: 154709512-154710827 |
| chr6: 33393592-33393908 | chr4: 154712073-154712706 |
| chr6: 33655966-33656238 | chr4: 154713537-154714240 |
| chr6: 41908745-41909711 | chr4: 155663809-155664315 |
| chr6: 42072032-42072701 | chr4: 156129168-156130209 |
| chr6: 46655262-46656738 | chr4: 158143296-158144053 |
| chr6: 50682334-50683214 | chr4: 169799086-169799625 |
| chr6: 50791110-50791573 | chr4: 174422024-174422443 |
| chr6: 55039170-55039392 | chr4: 174427891-174428192 |
| chr6: 99275763-99276038 | chr4: 174437914-174438346 |
| chr6: 101846766-101847135 | chr4: 174439812-174440249 |
| chr6: 108485671-108490539 | chr4: 174448333-174448845 |
| chr6: 108491033-108491410 | chr4: 174450046-174451469 |
| chr6: 108497595-108497996 | chr4: 174451828-174452962 |
| chr6: 117198089-117198705 | chr4: 174459200-174460054 |
| chr6: 117591533-117592279 | chr4: 185937242-185937750 |
| chr6: 134210639-134211218 | chr4: 187219320-187219745 |
| chr6: 134638797-134639021 | chr4: 188916605-188916876 |
| chr6: 13 7242315-137245442 | chr4: 190938106-190938848 |
| chr6: 137814355-137815202 | chr4: 190939801-190940591 |
| chr6: 138745348-138745593 | chr5: 1874907-1879032 |
| chr7: 1362811-1363643 | chr5: 2738953-2741237 |
| chr7: 6590563-6590957 | chr5: 3590644-3592000 |
| chr7: 6661875-6662695 | chr5: 3594467-3603054 |
| chr7: 19145872-19146256 | chr5: 11384681-11385521 |
| chr7: 20370003-20371504 | chr5: 31193952-31194419 |
| chr7: 20830567-20830817 | chr5: 45695394-45696510 |
| chr7: 26415746-26416891 | chr5: 50685453-50686148 |
| chr7: 27146069-27146600 | chr5: 54519054-54519628 |
| chr7: 27182613-27185562 | chr5: 63255044-63255407 |
| chr7: 27227520-27229043 | chr5: 72526203-72526497 |
| chr7: 27278945-27279469 | chr5: 72594147-72595808 |
| chr7: 27282086-27283136 | chr5: 72676120-72678421 |
| chr7: 30721372-30722445 | chr5: 76923 887-76924502 |
| chr7: 37955622-37956555 | chr5: 76936126-76936984 |
| chr7: 49813008-49815752 | chr5: 77140542-77140914 |
| chr7: 56355508-56355798 | chr5: 77146998-77147785 |
| chr7: 87563342-87564571 | chr5: 77253832-77254049 |
| chr7: 90893567-90896683 | chr5: 77268350-77268787 |
| chr7: 95225503-95226194 | chr5: 87968635-87968907 |
| chr7: 96650221-96651551 | chr5: 87980878-87981272 |
| chr7: 96651963-96652246 | chr5: 87985470-87985810 |
| chr7: 97841636-97842005 | chr5: 88185224-88185589 |
| chr7: 113724924-113727795 | chr5: 115697134-115697589 |
| chr7: 130790358-130792773 | chr5: 12243 0676-122431443 |
| chr7: 136553854-136556194 | chr5: 134385967-134386370 |
| chr7: 155595692-155599414 | chr5: 140346105-140346931 |
| chr7: 155604725-155605095 | chr5: 140787447-140788044 |
| chr7: 156795355-156799394 | chr5: 140864527-140864748 |
| chr8: 21905461-21905757 | chr5: 146888750-146889840 |
| chr8: 25900562-25905842 | chr5: 148033472-148034080 |
| chr8: 55366180-55367628 | chr5: 158478378-158478630 |
| chr8: 65710990-65711722 | chr5: 159399004-159399928 |
| chr8: 70981873-70984888 | chr5: 170735169-170739863 |
| chr8: 105478672-105479340 | chr5: 170741603-170742751 |
| chr8: 120428398-120429178 | chr5: 170743178-170744107 |
| chr8: 143545445-143546178 | chr5: 172110282-172111166 |
| chr8: 144808221-144810978 | chr5: 172659049-172660277 |
| chr8: 144990270-145002135 | chr5: 172660720-172661133 |
| chr9: 17906419-17907488 | chr5: 172661486-172662228 |
| chr9: 21970913-21971190 | chr5: 172672311 -172672971 |
| chr9: 22005887-22006229 | chr5: 174158680-174159729 |
| chr9: 86152353-86153777 | chr5: 175085004-175085756 |
| chr9: 95477296-95477708 | chr5: 178421225-178422337 |
| chr9: 96713326-96718186 | chr5: 180486154-180486892 |
| chr9: 97401286-97402067 | chr6: 1378445-1379318 |
| chr9: 102590742-102591303 | chr6: 1393049-1394170 |
| chr9: 112081402-112082905 | chr6: 1619093-1621094 |
| chr9: 120175253-120177496 | chr6: 4079052-4079443 |
| chr9: 122131086-122132214 | chr6: 5999149-5999787 |
| chr9: 124413512-124414193 | chr6: 10381558-10382354 |
| chr9: 124987743-124991086 | chr6: 10881846-10882051 |
| chr9: 126773246-126780953 | chr6: 26614013-26614851 |
| chr9: 129372737-129378106 | chr6: 27228100-27228364 |
| chr9: 129386112-129389231 | chr6: 29595298-29595795 |
| chr9: 131154346-131155923 | chr6: 30095173-30095610 |
| chr9: 132459587-132460017 | chr6: 30139718-30140263 |
| chr9: 133534534-133542394 | chr6: 33048416-33048814 |
| chr9: 135039673-135039978 | chr6: 35479388-35479678 |
| chr9: 135455164-135458586 | chr6: 37616722-37617179 |
| chr9: 135461934-135462909 | chr6: 38682949-38683265 |
| chr9: 135464586-135466240 | chr6: 41528266-41528900 |
| chr9: 139096665-139096993 | chr6: 42145847-42146053 |
| chr9: 139396205-139397040 | chr6: 42879279-42879623 |
| chrX: 673 52650-67352923 | chr6: 50787286-50788091 |
| chrX: 99891299-99891794 | chr6: 50810642-50810994 |
| chrX: 152612775-152613464 | chr6: 50813314-50813699 |
| chr1: 1474962-1475220 | chr6: 50818180-50818431 |
| chr1: 2979275-2980758 | chr6: 70992040-70992912 |
| chr1: 10764449-10764925 | chr6: 72298274-72298528 |
| chr1: 12123488-12124148 | chr6: 78172231-78174088 |
| chr1: 16860873-16862296 | chr6: 85472702-85474132 |
| chr1: 18964180-18964401 | chr6: 99290279-99290771 |
| chr1: 24229115-24229537 | chr6: 100038655-100039477 |
| chr1: 32052471-32052771 | chr6: 100897080-100897621 |
| chr1: 34642382-34643024 | chr6: 100903491-100903713 |
| chr1: 36549554-36549965 | chr6: 100905444-100905697 |
| chr1: 38219702-38220012 | chr6: 100905952-100906686 |
| chr1: 38461584-38461988 | chr6: 100914946-100915245 |
| chr1: 38941919-38942404 | chr6: 106429111-106429772 |
| chr1: 39044059-39044561 | chr6: 106433984-106434459 |
| chr1: 40769186-40769871 | chr6: 108495654-108495986 |
| chr1: 41284847-41285149 | chr6: 110299365-110301267 |
| chr1: 44031286-44031853 | chr6: 117869097-117869530 |
| chr1: 47009575-47010132 | chr6: 127441553-127441760 |
| chr1: 50880916-50881516 | chr6: 137809342-137810204 |
| chr1: 50881884-50882103 | chr6: 137816474-137817223 |
| chr1: 50892437-50893243 | chr6: 150335525-150336278 |
| chr1: 53527572-53528974 | chr6: 150358872-150359394 |
| chr1: 63795363-63796140 | chr6: 154360586-154361008 |
| chr1: 65991001-65991811 | chr6: 161188084-161188639 |
| chr1: 67218079-67218293 | chr6: 166579973-166583423 |
| chr1: 67773329-67773767 | chr6: 166666837-166667541 |
| chr1: 86621278-86622871 | chr6: 168841438-168841699 |
| chr1: 91183240-91184540 | chr6: 170732119-170732442 |
| chr1: 91185156-91185577 | chr7: 751712-752150 |
| chr1: 91190489-91192804 | chr7: 12151220-12151559 |
| chr1: 91300979-91301891 | chr7: 19184818-19185033 |
| chr1: 110610265-110613303 | chr7: 23287221-23287508 |
| chr1: 113265573-113265787 | chr7: 27134097-27134303 |
| chr1: 113286332-113287172 | chr7: 27147589-27148389 |
| chr1: 114695136-114696672 | chr7: 27198182-27198514 |
| chr1: 119526782-119527192 | chr7: 27203915-27206462 |
| chr1: 119529819-119530712 | chr7: 27260101-27260467 |
| chr1: 119543056-119543454 | chr7: 27291119-27292197 |
| chr1: 119549144-119551320 | chr7: 32110063-32110910 |
| chr1: 145075483-145075845 | chr7: 35296921-35298218 |
| chr1: 146552328-146552577 | chr7: 42267546-42267823 |
| chr1: 147782066-147782473 | chr7: 43152020-43153340 |
| chr1: 149332993-149333389 | chr7: 53286851-53287192 |
| chr1: 155147185-155147444 | chr7: 54612324-54612558 |
| chr1: 155264318-155265536 | chr7: 70596228-70598382 |
| chr1: 155290606-155291001 | chr7: 71800757-71802768 |
| chr1: 156863415-156863711 | chr7: 72838383-72838815 |
| chr1: 164545540-164545917 | chr7: 73894815-73895110 |
| chr1: 165324191-165326328 | chr7: 89747892-89749036 |
| chr1: 170630456-170630851 | chr7: 97361132-97363018 |
| chr1: 173638662-173639045 | chr7: 100075303-100075551 |
| chr1: 175568376-175568808 | chr7: 100817759-100817975 |
| chr1: 179544720-179545307 | chr7: 100823307-100823701 |
| chr1: 181287300-181287873 | chr7: 101005899-101007443 |
| chr1: 181452706-181453073 | chr7: 103085710-103086132 |
| chr1: 200009807-200010036 | chr7: 103968783-103969959 |
| chr1: 202162958-202163390 | chr7: 121940006-121940648 |
| chr1: 203044722-203045390 | chr7: 121950249-121950927 |
| chr1: 208132327-208133117 | chr7: 121956543-121957341 |
| chr1: 214153214-214153668 | chr7: 124404174-124404432 |
| chr1: 217310749-217311178 | chr7: 127990926-127992616 |
| chr1: 221050448-221050864 | chr7: 128555329-128556650 |
| chr1: 221060850-221061071 | chr7: 129422997-129423355 |
| chr1: 225865068-225865328 | chr7: 142494563-142495248 |
| chr1: 226127112-226127695 | chr7: 143582125-143582610 |
| chr1: 228785986-228786204 | chr7: 149389654-149389976 |
| chr1: 231296559-231297345 | chr7: 149744402-149746469 |
| chr1: 243646394-243646888 | chr7: 152621916-152622149 |
| chr10: 1778784-1780018 | chr7: 153748407-153750444 |
| chr10: 8076002-8077261 | chr7: 154001964-154002281 |
| chr10: 8077829-8078378 | chr7: 155164557-155167854 |
| chr10: 15761423-15762101 | chr7: 155174128-155175248 |
| chr10: 16561604-16563822 | chr7: 155241323-155243757 |
| chr10: 22623350-22625875 | chr7: 155258827-155261403 |
| chr10: 22634000-22634862 | chr7: 155302253-155303158 |
| chr10: 22764708-22767050 | chr7: 156409023-156409294 |
| chr10: 23461300-23461610 | chr7: 156409577-156409865 |
| chr10: 23462224-23463889 | chr7: 156801418-156801632 |
| chr10: 23480697-23482455 | chr7: 156871054-156871297 |
| chr10: 23983366-23984978 | chr7: 158936507-158938492 |
| chr10: 26504383-26507434 | chr8: 4848968-4852635 |
| chr10: 27547668-27548402 | chr8: 9760750-9761643 |
| chr10: 43428167-43429460 | chr8: 9762661-9764748 |
| chr10: 48438411-48439320 | chr8: 11536767-11538961 |
| chr10: 63212495-63213009 | chr8: 11557852-11558252 |
| chr10: 71331449-71331691 | chr8: 11565217-11567212 |
| chr10: 75407413-75407706 | chr8: 21644908-21647845 |
| chr10: 76573195-76573507 | chr8: 23562475-23565175 |
| chr10: 94180315-94180754 | chr8: 23567180-23567678 |
| chr10: 94455524-94455896 | chr8: 24812946-24814299 |
| chr10: 94828102-94829040 | chr8: 26721642-26724566 |
| chr10: 99789614-99791320 | chr8: 37822486-37824008 |
| chr10: 100992156-100992687 | chr8: 41424341-41425300 |
| chr10: 101282725-101282934 | chr8: 49468683-49468959 |
| chr10: 101290025-101290338 | chr8: 50822270-50822860 |
| chr10: 102279162-102279730 | chr8: 53851701-53854426 |
| chr10: 102475276-102475579 | chr8: 55370170-55372525 |
| chr10: 102891010-102891794 | chr8: 55378928-55380186 |
| chr10: 102905714-102906693 | chr8: 57358126-57359415 |
| chr10: 102996034-102996646 | chr8: 65281903-65283043 |
| chr10: 103043990-103044480 | chr8: 65286067-65286659 |
| chr10: 108923780-108924805 | chr8: 65290108-65290946 |
| chr10: 109674196-109674964 | chr8: 68864584-68864946 |
| chr10: 110671724-110672326 | chr8: 72468560-72469561 |
| chr10: 111216604-111217083 | chr8: 85096759-85097247 |
| chr10: 118030732-118034230 | chr8: 86350765-86351196 |
| chr10: 118892161-118892639 | chr8: 87081653-87082046 |
| chr10: 118893527-118894432 | chr8: 97169731-97170432 |
| chr10: 119494493-119494991 | chr8: 97171805-97172022 |
| chr10: 120353692-120355821 | chr8: 98289604-98290404 |
| chr10: 121577529-121578385 | chr8: 99960497-99961438 |
| chr10: 123922850-123923542 | chr8: 99984584-99985072 |
| chr10: 124901907-124902617 | chr8: 99985733-99986983 |
| chr10: 125425495-125426642 | chr8: 101117922-101118693 |
| chr10: 125650820-125651373 | chr8: 130995921-130996149 |
| chr10: 125732220-125732843 | chr8: 132052203-132054749 |
| chr10: 130338695-130338994 | chr8: 139508795-139509774 |
| chr10: 130508443-130508658 | chr8: 142528185-142529029 |
| chr10: 134597357-134602649 | chr8: 145103285-145108027 |
| chr11: 626728-628037 | chr8: 145925410-145926101 |
| chr11: 636435-636668 | chr9: 969529-973276 |
| chr11: 636906-640628 | chr9: 16726859-16727273 |
| chr11: 2890388-2891337 | chr9: 19788215-19789288 |
| chr11: 14995128-14995908 | chr9: 23820691-23822135 |
| chr11: 20618197-20619920 | chr9: 23850910-23851522 |
| chr11: 27743472-27744564 | chr9: 32782936-32783625 |
| chr11: 31827696-31827921 | chr9: 36739534-36739782 |
| chr11: 31841315-31842003 | chr9: 37002489-37002957 |
| chr11: 31847132-31847958 | chr9: 77112712-77113583 |
| chr11: 43568921-43569854 | chr9: 77113709-77113927 |
| chr11: 44325657-44326517 | chr9: 79633326-79636030 |
| chr11: 60718428-60718888 | chr9: 79637814-79638169 |
| chr11: 64478843-64479598 | chr9: 91792662-91793611 |
| chr11: 64815040-64815722 | chr9: 96108466-96108992 |
| chr11: 65409636-65410127 | chr9: 96710811-96711717 |
| chr11: 65816404-65816665 | chr9: 98111364-98112362 |
| chr11: 68622108-68622339 | chr9: 100610696-100611517 |
| chr11: 70508328-70508617 | chr9: 100619984-100620192 |
| chr11: 71952112-71952528 | chr9: 104499849-104501076 |
| chr11: 88241710-88242562 | chr9: 115822071-115823416 |
| chr11: 89224416-89224718 | chr9: 120507227-120507642 |
| chr11: 105481126-105481422 | chr9: 123656750-123656972 |
| chr11: 115630398-115631117 | chr9: 134429866-134430491 |
| chr11: 119293320-119293943 | chr9: 136294738-136295236 |
| chr11: 123066517-123066986 | chr9: 137967110-137967727 |
| chr11: 128419198-128419513 | chr9: 139715663-139716441 |
| chr11: 128694084-128694688 | |
| chr11: 131780328-131781532 | |
| chr11: 132813562-132814395 | |
| chr11: 132934059-132934291 | |
| chr11: 132952538-132953307 | |
| chr11: 133994709-133995090 | |
| chr12: 186863-187610 | |
| chr12: 3308812-3310270 | |
| chr12: 5153012-5154346 | |
| chr12: 14134626-14135242 | |
| chr12: 41086522-41087102 | |
| chr12: 48399168-48399372 | |
| chr12: 52115410-52115679 | |
| chr12: 52408381-52408675 | |
| chr12: 52652018-52652743 | |
| chr12: 53107912-53108471 | |
| chr12: 53359192-53359507 | |
| chr12: 54071053-54071265 | |
| chr12: 54321301-54321721 | |
| chr12: 54354529-54355491 | |
| chr12: 54359658-54359906 | |
| chr12: 54424610-54425173 | |
| chr12: 65218245-65219143 | |
| chr12: 65514878-65515863 | |
| chr12: 72665683-72667551 | |
| chr12: 81102034-81102716 | |
| chr12: 81471569-81472119 | |

TABLE 3
Additional Example CGIs

| | | |
|---|---|---|
| chr1: 1072370-1072847 | chr11: 65190825-65191058 | chr16: 72821141-72821592 |
| chr1: 10895896-10896117 | chr11: 65222491-65222750 | chr16: 73099813-73100791 |
| chr1: 109203594-109204378 | chr11: 65341621-65342501 | chr16: 743925-745943 |
| chr1: 1093212-1093476 | chr11: 65343330-65343849 | chr16: 78079753-78080166 |
| chr1: 110185962-110186164 | chr11: 65553750-65555573 | chr16: 80574742-80575090 |
| chr1: 110626529-110627484 | chr11: 65779312-65779767 | chr16: 80965953-80966478 |
| chr1: 110880395-110880624 | chr11: 66034752-66035054 | chr16: 84029457-84029710 |
| chr1: 111505882-111507007 | chr11: 66035217-66035447 | chr16: 84328520-84328720 |
| chr1: 111746338-111747303 | chr11: 66049751-66050229 | chr16: 84346477-84346931 |
| chr1: 113044411-113044992 | chr11: 66314208-66314455 | chr16: 84401958-84402497 |
| chr1: 113392143-113392807 | chr11: 66335576-66336151 | chr16: 85171020-85171323 |
| chr1: 113497987-113498206 | chr11: 67232299-67232558 | chr16: 85783863-85785131 |
| chr1: 1141671-1142150 | chr11: 67770427-67771629 | chr16: 85863382-85863601 |
| chr1: 11538670-11540342 | chr11: 67806252-67806611 | chr16: 85932122-85932942 |
| chr1: 116694665-116694983 | chr11: 68611251-68611807 | chr16: 86546360-86546632 |
| chr1: 116710838-116711260 | chr11: 69258150-69258544 | chr16: 87902455-87903460 |
| chr1: 11710460-11710788 | chr11: 69924339-69925197 | chr16: 88292764-88293010 |
| chr1: 11779567-11780016 | chr11: 705795-706534 | chr16: 88716990-88717606 |
| chr1: 118727817-118728097 | chr11: 70962174-70964161 | chr16: 88803803-88804112 |
| chr1: 120835962-120839391 | chr11: 71954817-71955659 | chr16: 88850205-88850537 |
| chr1: 12655927-12656248 | chr11: 720562-721369 | chr16: 89070647-89070904 |
| chr1: 1362955-1363299 | chr11: 72301303-72301746 | chr16: 89267824-89268087 |
| chr1: 1370768-1371449 | chr11: 72463093-72463717 | chr16: 89268493-89268865 |
| chr1: 13839506-13840613 | chr11: 72492282-72492644 | chr16: 89323281-89323661 |
| chr1: 13909607-13909842 | chr11: 74022429-74022703 | chr16: 89632593-89632799 |
| chr1: 14026482-14027200 | chr11: 75236190-75237781 | chr16: 90014251-90014613 |
| chr1: 14219351-14219737 | chr11: 75917272-75917926 | chr17: 10632790-10633490 |
| chr1: 146556313-146556676 | chr11: 77122737-77123088 | chr17: 11501632-11502328 |
| chr1: 14924611-14925993 | chr11: 78673008-78673213 | chr17: 1163342-1163773 |
| chr1: 149605515-149605903 | chr11: 789872-790133 | chr17: 12692738-12693690 |
| chr1: 150254366-150254637 | chr11: 8102359-8102913 | chr17: 1390457-1390786 |
| chr1: 150266477-150266689 | chr11: 826942-827625 | chr17: 1395120-1395372 |
| chr1: 151300523-151300724 | chr11: 8284103-8285032 | chr17: 14212364-14212788 |
| chr1: 151445872-151446142 | chr11: 86382696-86383586 | chr17: 15244706-15245126 |
| chr1: 151693992-151694282 | chr11: 87908244-87908614 | chr17: 15466360-15466843 |
| chr1: 151812254-151812525 | chr11: 9025096-9026315 | chr17: 1546743-1547324 |
| chr1: 151966633-151966893 | chr11: 93583375-93583717 | chr17: 1551731-1553249 |
| chr1: 152079998-152081705 | chr11: 94473536-94474338 | chr17: 15847758-15849513 |
| chr1: 154298206-154298544 | chr11: 94501367-94502696 | chr17: 16283928-16284768 |
| chr1: 154732823-154733436 | chr11: 9634970-9636065 | chr17: 17685017-17687240 |
| chr1: 154971871-154972404 | chr11: 9779593-9780470 | chr17: 18965478-18965728 |
| chr1: 155043413-155043922 | chr11: 98891544-98891821 | chr17: 2627241-2628302 |
| chr1: 155830196-155830489 | chr12: 103350090-103350422 | chr17: 26578273-26578682 |
| chr1: 156051240-156051461 | chr12: 103351580-103352695 | chr17: 26645291-26645614 |
| chr1: 156616554-156616946 | chr12: 103359249-103359629 | chr17: 26698360-26699557 |
| chr1: 156646293-156647260 | chr12: 104850254-104852395 | chr17: 26711384-26712311 |
| chr1: 156814882-156815792 | chr12: 105478090-105478517 | chr17: 27038085-27038919 |
| chr1: 156893520-156894232 | chr12: 106532107-106533696 | chr17: 27332269-27333188 |
| chr1: 157963541-157963947 | chr12: 107711604-107714107 | chr17: 27503599-27504014 |
| chr1: 158119489-158119704 | chr12: 108297427-108297743 | chr17: 27942533-27945388 |
| chr1: 159141203-159141718 | chr12: 109162409-109162722 | chr17: 27949430-27950277 |
| chr1: 15929824-15930289 | chr12: 109729573-109729826 | chr17: 29298047-29298606 |
| chr1: 160040129-160040668 | chr12: 110150048-110150262 | chr17: 29718231-29719291 |
| chr1: 16085148-16085862 | chr12: 110156268-110156496 | chr17: 29814615-29815662 |
| chr1: 161228478-161229028 | chr12: 111471961-111473546 | chr17: 30593199-30594033 |
| chr1: 162760251-162760722 | chr12: 112204499-112204979 | chr17: 30845904-30846702 |
| chr1: 162792177-162792574 | chr12: 115104849-115105548 | chr17: 32953154-32953801 |
| chr1: 16543684-16544307 | chr12: 115120775-115122945 | chr17: 33787402-33787845 |
| chr1: 166134259-166136448 | chr12: 115135926-115136350 | chr17: 33814235-33814947 |
| chr1: 167789397-167789647 | chr12: 115889598-115889995 | chr17: 34091137-34091919 |
| chr1: 17033769-17034728 | chr12: 116354788-116355187 | chr17: 3438842-3439046 |
| chr1: 171810468-171811325 | chr12: 116946196-116946607 | chr17: 35060323-35060692 |
| chr1: 179555402-179555770 | chr12: 117316390-117317611 | chr17: 35303285-35303572 |
| chr1: 180881317-180882592 | chr12: 117536291-117537421 | chr17: 36102034-36104766 |
| chr1: 182584178-182584545 | chr12: 120031495-120033212 | chr17: 36105335-36105583 |
| chr1: 184633224-184633663 | chr12: 120799373-120799912 | chr17: 36575500-36575782 |
| chr1: 1875618-1875877 | chr12: 122277302-122277539 | chr17: 36584421-36585453 |
| chr1: 18971730-18972097 | chr12: 122667649-122668038 | chr17: 36728634-36729284 |
| chr1: 19970256-19971923 | chr12: 123380334-123380894 | chr17: 37365987-37366539 |
| chr1: 200860077-200860576 | chr12: 126018101-126018365 | chr17: 37856449-37856891 |
| chr1: 200992283-200992839 | chr12: 128850550-128850755 | chr17: 38020382-38020645 |
| chr1: 201368561-201369032 | chr12: 129787736-129788160 | chr17: 38347534-38347765 |
| chr1: 201450881-201451105 | chr12: 130526916-130527117 | chr17: 38497528-38498963 |
| chr1: 201475886-201476516 | chr12: 13152820-13153084 | chr17: 38501397-38501839 |
| chr1: 201708788-201709429 | chr12: 132312440-132315739 | chr17: 39683909-39684599 |
| chr1: 202936046-202936252 | chr12: 132689881-132690197 | chr17: 39705046-39705332 |
| chr1: 203456785-203457059 | chr12: 132690340-132690571 | chr17: 40250273-40250591 |
| chr1: 203598472-203598853 | chr12: 133463808-133464858 | chr17: 40332598-40333471 |
| chr1: 204159599-204159833 | chr12: 14927292-14928023 | chr17: 40440189-40441014 |
| chr1: 204797611-204797930 | chr12: 175667-176400 | chr17: 40805675-40805957 |
| chr1: 20512361-20512797 | chr12: 1770702-1771476 | chr17: 40912817-40913553 |
| chr1: 205537752-205538443 | chr12: 1905278-1906765 | chr17: 40932330-40933299 |
| chr1: 206223538-206224028 | chr12: 20521617-20523122 | chr17: 41723220-41723826 |
| chr1: 2064629-2064855 | chr12: 21680409-21680982 | chr17: 41791111-41791476 |
| chr1: 206730398-206730908 | chr12: 21810489-21810766 | chr17: 41984149-41985012 |
| chr1: 20810463-20813511 | chr12: 22486836-22488666 | chr17: 42015422-42015707 |
| chr1: 209848444-209849428 | chr12: 24714957-24716243 | chr17: 42015844-42016069 |
| chr1: 209979317-209979666 | chr12: 26348261-26349130 | chr17: 42030174-42030941 |
| chr1: 210465710-210466212 | chr12: 2800140-2801062 | chr17: 42061047-42061643 |
| chr1: 211306668-211307675 | chr12: 28127891-28128575 | chr17: 42082028-42084972 |
| chr1: 211688462-211689104 | chr12: 29935996-29937433 | chr17: 42091713-42091948 |
| chr1: 213123648-213125092 | chr12: 3862069-3862606 | chr17: 42092144-42092432 |
| chr1: 214161198-214161415 | chr12: 4273820-4274491 | chr17: 42287693-42288392 |
| chr1: 2144200-2144497 | chr12: 4378367-4382222 | chr17: 42392324-42393079 |
| chr1: 215256052-215256636 | chr12: 4383194-4384405 | chr17: 42402788-42403266 |
| chr1: 219347110-219347572 | chr12: 49318487-49319476 | chr17: 44026528-44026738 |
| chr1: 220960017-220960603 | chr12: 49363665-49364443 | chr17: 44848309-44849912 |
| chr1: 2222199-2222569 | chr12: 49390618-49392441 | chr17: 45400875-45401440 |
| chr1: 225117221-225117781 | chr12: 49487964-49488202 | chr17: 45928212-45928710 |
| chr1: 226270724-226271841 | chr12: 49688874-49691360 | chr17: 46089637-46089851 |
| chr1: 22668639-22668862 | chr12: 49735720-49736875 | chr17: 46114574-46115059 |
| chr1: 226736355-226737412 | chr12: 50297581-50297988 | chr17: 46507345-46507778 |
| chr1: 227729516-227730492 | chr12: 50349080-50349525 | chr17: 46655216-46655604 |
| chr1: 228565950-228567121 | chr12: 51785280-51785821 | chr17: 46687528-46688730 |
| chr1: 230561104-230562702 | chr12: 51818461-51819166 | chr17: 46710813-46711419 |
| chr1: 231175063-231176317 | chr12: 52444554-52445421 | chr17: 46719361-46720234 |
| chr1: 231176786-231177009 | chr12: 52545938-52546363 | chr17: 46723732-46724383 |
| chr1: 232941055-232941707 | chr12: 52701963-52702560 | chr17: 46755566-46756006 |
| chr1: 233749374-233750314 | chr12: 53267860-53268290 | chr17: 46827436-46827641 |
| chr1: 236687072-236687608 | chr12: 53273232-53273498 | chr17: 47209812-47210740 |
| chr1: 23750509-23751663 | chr12: 53297443-53297824 | chr17: 47572346-47575316 |
| chr1: 23884843-23885087 | chr12: 53441385-53441706 | chr17: 47647377-47647660 |
| chr1: 240254960-240257063 | chr12: 53448009-53448406 | chr17: 47967874-47968409 |
| chr1: 244012713-244013245 | chr12: 53613717-53615103 | chr17: 48619112-48619794 |
| chr1: 244213398-244213619 | chr12: 53718633-53719778 | chr17: 49021857-49022279 |
| chr1: 2460761-2462010 | chr12: 54332806-54333731 | chr17: 4981358-4981979 |
| chr1: 24648203-24648985 | chr12: 54343623-54343848 | chr17: 52977867-52978307 |
| chr1: 24739858-24740262 | chr12: 54346778-54347101 | chr17: 53315619-53316198 |
| chr1: 2477564-2478363 | chr12: 54387826-54388732 | chr17: 53342199-53343061 |
| chr1: 26490523-26491015 | chr12: 54389114-54389520 | chr17: 54674159-54674366 |
| chr1: 26686517-26687281 | chr12: 54393375-54394648 | chr17: 54910497-54912470 |
| chr1: 27338880-27339441 | chr12: 54399413-54399654 | chr17: 55939089-55939591 |
| chr1: 27854417-27854963 | chr12: 54399907-54400495 | chr17: 56832962-56833986 |
| chr1: 27894928-27895524 | chr12: 54408427-54408713 | chr17: 58228062-58228361 |
| chr1: 27960568-27961023 | chr12: 54784901-54785238 | chr17: 58498712-58499332 |
| chr1: 27986306-27986808 | chr12: 5541211-5542973 | chr17: 59473061-59483266 |
| chr1: 29101791-29102069 | chr12: 56522944-56523285 | chr17: 59539363-59539834 |
| chr1: 2929156-2929376 | chr12: 56881402-56882702 | chr17: 5973316-5974449 |
| chr1: 31158010-31158261 | chr12: 58013172-58013652 | chr17: 60704543-60705943 |
| chr1: 31380845-31381078 | chr12: 58021295-58022037 | chr17: 60729646-60730269 |
| chr1: 32169538-32169869 | chr12: 58258987-58259496 | chr17: 60885409-60885857 |
| chr1: 32180132-32180487 | chr12: 59989652-59990507 | chr17: 61523001-61524470 |
| chr1: 32226147-32226535 | chr12: 63025589-63026213 | chr17: 61615393-61616035 |
| chr1: 32237828-32238661 | chr12: 6419604-6420024 | chr17: 6460072-6460302 |
| chr1: 3239916-3240261 | chr12: 6472661-6473322 | chr17: 64961008-64962321 |
| chr1: 32410189-32410630 | chr12: 6492890-6493522 | chr17: 68164914-68165720 |
| chr1: 32892429-32892835 | chr12: 65672392-65673250 | chr17: 6945390-6947521 |
| chr1: 3310103-3311035 | chr12: 6664426-6665336 | chr17: 7108305-7108654 |
| chr1: 33219428-33220028 | chr12: 71003625-71004057 | chr17: 71639029-71641670 |
| chr1: 33358470-33359449 | chr12: 8123347-8123561 | chr17: 7164286-7166245 |
| chr1: 33438459-33438979 | chr12: 81330609-81331514 | chr17: 71897971-71898420 |
| chr1: 33938026-33938328 | chr12: 82152321-82152674 | chr17: 72270302-72270512 |
| chr1: 3447450-3447950 | chr12: 8849964-8851403 | chr17: 72352828-72353798 |
| chr1: 35331704-35332409 | chr12: 96883287-96883737 | chr17: 72442928-72443194 |
| chr1: 35350879-35351854 | chr12: 98850691-98851002 | chr17: 72449983-72450617 |
| chr1: 35394748-35396206 | chr12: 99288223-99289374 | chr17: 72667196-72667710 |
| chr1: 36042433-36043444 | chr13: 100631753-100635526 | chr17: 72732849-72733545 |
| chr1: 3662964-3664085 | chr13: 102068117-102069258 | chr17: 72848167-72848901 |
| chr1: 36771831-36773009 | chr13: 107186469-107189024 | chr17: 72889350-72890060 |
| chr1: 3688554-3689684 | chr13: 110958892-110960590 | chr17: 72931730-72932601 |
| chr1: 37498378-37500624 | chr13: 110960925-110961143 | chr17: 73030677-73031160 |
| chr1: 38229839-38230888 | chr13: 113548644-113549127 | chr17: 73083867-73084495 |
| chr1: 41847265-41849204 | chr13: 113597554-113598303 | chr17: 73520957-73522540 |
| chr1: 43832815-43833073 | chr13: 113622738-113623660 | chr17: 74496993-74497632 |
| chr1: 44401758-44402423 | chr13: 113807289-113807865 | chr17: 74706466-74707067 |
| chr1: 44871110-44874047 | chr13: 114017867-114018463 | chr17: 75524590-75525152 |
| chr1: 44883137-44884272 | chr13: 20175967-20176766 | chr17: 7608377-7608956 |
| chr1: 46767426-46769036 | chr13: 20702852-20703122 | chr17: 76127522-76128406 |
| chr1: 46859725-46860291 | chr13: 20806017-20806867 | chr17: 77093292-77093805 |
| chr1: 46913787-46914343 | chr13: 23489631-23490058 | chr17: 77783810-77789842 |
| chr1: 47489227-47489633 | chr13: 26760422-26760892 | chr17: 78977614-78978447 |
| chr1: 47690981-47691727 | chr13: 28491489-28492518 | chr17: 79366807-79374742 |
| chr1: 47915640-47915952 | chr13: 28501860-28502090 | chr17: 79448219-79448827 |
| chr1: 47998900-47999517 | chr13: 28542222-28543544 | chr17: 79485600-79486913 |
| chr1: 48058794-48059230 | chr13: 28554428-28555065 | chr17: 79614851-79615559 |
| chr1: 48190757-48190992 | chr13: 29105412-29105676 | chr17: 79917287-79920022 |
| chr1: 48449871-48450144 | chr13: 29292671-29293329 | chr17: 80329067-80330208 |
| chr1: 48462132-48462976 | chr13: 30995686-30995947 | chr17: 8054551-8055835 |
| chr1: 48937305-48937683 | chr13: 31019861-31020137 | chr17: 9142981-9143839 |
| chr1: 49242372-49242810 | chr13: 31377146-31377493 | chr18: 10131854-10132080 |
| chr1: 50513645-50514320 | chr13: 36705041-36705707 | chr18: 12271658-12271920 |
| chr1: 50798668-50799536 | chr13: 36871668-36872059 | chr18: 12407716-12408242 |
| chr1: 53386618-53387523 | chr13: 36919738-36921004 | chr18: 13136597-13137564 |
| chr1: 55446088-55446846 | chr13: 37005582-37006453 | chr18: 13824002-13824215 |
| chr1: 57110664-57111337 | chr13: 37247950-37248463 | chr18: 14132001-14132482 |
| chr1: 57887964-57890637 | chr13: 43597505-43597856 | chr18: 14430639-14431567 |
| chr1: 59280952-59281194 | chr13: 44359859-44361696 | chr18: 19928417-19929329 |
| chr1: 60280625-60281048 | chr13: 44453208-44454295 | chr18: 20139485-20140246 |
| chr1: 6086245-6086494 | chr13: 45885755-45886103 | chr18: 21199432-21199798 |
| chr1: 61508643-61509282 | chr13: 50070023-50070719 | chr18: 21269270-21270349 |
| chr1: 61519353-61519971 | chr13: 53174029-53174674 | chr18: 22006311-22007007 |
| chr1: 6208717-6209039 | chr13: 53424942-53425995 | chr18: 24443201-24443458 |
| chr1: 6241032-6241251 | chr13: 67804594-67805459 | chr18: 25755415-25758175 |
| chr1: 6265826-6266778 | chr13: 88323570-88324640 | chr18: 2846566-2848175 |
| chr1: 6301696-6302856 | chr13: 95201561-95202293 | chr18: 28681321-28682455 |
| chr1: 6484504-6485327 | chr13: 95354190-95355184 | chr18: 2905951-2907348 |
| chr1: 6507208-6509186 | chr13: 95619884-95620977 | chr18: 29077553-29078526 |
| chr1: 6545144-6545559 | chr13: 96204692-96205496 | chr18: 31802359-31803792 |
| chr1: 65468273-65468828 | chr13: 98794399-98796241 | chr18: 31804481-31804875 |
| chr1: 68696640-68697628 | chr14: 100204041-100204262 | chr18: 34823799-34824017 |
| chr1: 70032968-70034495 | chr14: 100625320-100626461 | chr18: 3498942-3499358 |
| chr1: 72748472-72749736 | chr14: 100680270-100680947 | chr18: 42258984-42260795 |
| chr1: 76080455-76080808 | chr14: 101012841-101013117 | chr18: 43355319-43355698 |
| chr1: 76540148-76540653 | chr14: 101121009-101121365 | chr18: 43913346-43914477 |
| chr1: 77333112-77334534 | chr14: 102247661-102248279 | chr18: 44526867-44527137 |
| chr1: 8002409-8002699 | chr14: 103010785-103011625 | chr18: 45662315-45663520 |
| chr1: 8013994-8014651 | chr14: 103389017-103390248 | chr18: 47087076-47087446 |
| chr1: 805199-805628 | chr14: 103394398-103397070 | chr18: 47087880-47088528 |
| chr1: 82265999-82269048 | chr14: 103589354-103590246 | chr18: 48085839-48087589 |
| chr1: 8277196-8277822 | chr14: 103739579-103740892 | chr18: 5237724-5238674 |
| chr1: 84326268-84326838 | chr14: 105293561-105293879 | chr18: 52495605-52496007 |
| chr1: 85462586-85463435 | chr14: 105309691-105310500 | chr18: 52626518-52626849 |
| chr1: 87617154-87617417 | chr14: 105330515-105332186 | chr18: 53447075-53447842 |
| chr1: 87617693-87617986 | chr14: 105512056-105512307 | chr18: 5542848-5543054 |
| chr1: 90308840-90309606 | chr14: 105662778-105663403 | chr18: 55862654-55862873 |
| chr1: 9258566-9258956 | chr14: 105952604-105954296 | chr18: 56932714-56932994 |
| chr1: 94702691-94703344 | chr14: 21100839-21101043 | chr18: 56934834-56936827 |
| chr1: 95285603-95286319 | chr14: 21537083-21537355 | chr18: 5890293-5891682 |
| chr1: 9749296-9750228 | chr14: 21539712-21540045 | chr18: 59992070-59993556 |
| chr1: 999679-999911 | chr14: 23355739-23356402 | chr18: 63417776-63418640 |
| chr10: 100227439-100227832 | chr14: 24457865-24458100 | chr18: 65183238-65184014 |
| chr10: 101293016-101293238 | chr14: 24550318-24551148 | chr18: 70208975-70211790 |
| chr10: 102416497-102416716 | chr14: 24835811-24836220 | chr18: 72123607-72124717 |
| chr10: 102430698-102431119 | chr14: 24837873-24838324 | chr18: 74514041-74514292 |
| chr10: 102440601-102441011 | chr14: 24898780-24899288 | chr18: 74843361-74845426 |
| chr10: 102469807-102470341 | chr14: 25518425-25519612 | chr18: 7566558-7568830 |
| chr10: 102501701-102502232 | chr14: 27066314-27066578 | chr18: 77724353-77724836 |
| chr10: 102586126-102588109 | chr14: 32670418-32671092 | chr18: 811650-812709 |
| chr10: 102590123-102590402 | chr14: 34419509-34420438 | chr18: 8367274-8367599 |
| chr10: 102778605-102778922 | chr14: 37641233-37641879 | chr18: 9333897-9334244 |
| chr10: 102792043-102792266 | chr14: 38052646-38054166 | chr19: 10077032-10077444 |
| chr10: 102807775-102808271 | chr14: 38057139-38057798 | chr19: 10120751-10121231 |
| chr10: 102809888-102810282 | chr14: 38060842-38062119 | chr19: 10526983-10527755 |
| chr10: 102882978-102883551 | chr14: 38063664-38065665 | chr19: 10529628-10532004 |
| chr10: 103326283-103326712 | chr14: 38067447-38069207 | chr19: 1068439-1068764 |
| chr10: 103538816-103539988 | chr14: 45431629-45432115 | chr19: 10697787-10698099 |
| chr10: 103985853-103986246 | chr14: 52780688-52781969 | chr19: 1074727-1075071 |
| chr10: 104000255-104001741 | chr14: 54413305-54413764 | chr19: 11353961-11354362 |
| chr10: 105211951-105212306 | chr14: 55595698-55596692 | chr19: 11491560-11492740 |
| chr10: 106028543-106029047 | chr14: 58862542-58863209 | chr19: 11529214-11529588 |
| chr10: 110225928-110226465 | chr14: 60097209-60097553 | chr19: 11529723-11529966 |
| chr10: 11059443-11060524 | chr14: 60794577-60794867 | chr19: 11531279-11531590 |
| chr10: 11207179-11207980 | chr14: 61114103-61116552 | chr19: 11533199-11533619 |
| chr10: 113943284-113943657 | chr14: 61746805-61748141 | chr19: 11689418-11689768 |
| chr10: 115860680-115861093 | chr14: 61787881-61789467 | chr19: 11909403-11909841 |
| chr10: 116391467-116391827 | chr14: 62583680-62584279 | chr19: 11959578-11960064 |
| chr10: 116527525-116528474 | chr14: 67878535-67879167 | chr19: 11998805-11999131 |
| chr10: 116852262-116854094 | chr14: 70041214-70041662 | chr19: 12076030-12076366 |
| chr10: 118976050-118976706 | chr14: 73704055-73705106 | chr19: 12098572-12098950 |
| chr10: 119293918-119297675 | chr14: 73706300-73706677 | chr19: 12146031-12146649 |
| chr10: 122708511-122708899 | chr14: 73957751-73958889 | chr19: 12163327-12163560 |
| chr10: 122739053-122739391 | chr14: 74036128-74036662 | chr19: 12175461-12176057 |
| chr10: 124638744-124639793 | chr14: 74058401-74059116 | chr19: 12266999-12267686 |
| chr10: 125851152-125853233 | chr14: 74100344-74101230 | chr19: 12305553-12306304 |
| chr10: 126135810-126138896 | chr14: 75389713-75390133 | chr19: 12444033-12444548 |
| chr10: 133879006-133879255 | chr14: 75894309-75895469 | chr19: 12476050-12476372 |
| chr10: 134973511-134974311 | chr14: 76819146-76819379 | chr19: 12943347-12943661 |
| chr10: 135075002-135075452 | chr14: 88789601-88790493 | chr19: 12978360-12978785 |
| chr10: 135089970-135090491 | chr14: 89493136-89494382 | chr19: 13207376-13207621 |
| chr10: 13933483-13934184 | chr14: 90167783-90168352 | chr19: 13208344-13210525 |
| chr10: 17270431-17272617 | chr14: 91883362-91883566 | chr19: 1324970-1325349 |
| chr10: 17495408-17496721 | chr14: 92302202-92303078 | chr19: 13266390-13267030 |
| chr10: 18429126-18430234 | chr14: 92413582-92414324 | chr19: 1401093-1401784 |
| chr10: 20104452-20106105 | chr14: 96505312-96506402 | chr19: 14168143-14168512 |
| chr10: 20106644-20106946 | chr14: 96890431-96890633 | chr19: 14542769-14543102 |
| chr10: 21462129-21463808 | chr14: 97685060-97685946 | chr19: 1455227-1455544 |
| chr10: 22518027-22518334 | chr14: 99711840-99713431 | chr19: 1456078-1456347 |
| chr10: 25241111-25241900 | chr14: 99739802-99740920 | chr19: 1456886-1457312 |
| chr10: 25463757-25465639 | chr15: 100880958-100882438 | chr19: 1503512-1503791 |
| chr10: 26222922-26224022 | chr15: 100890243-100890494 | chr19: 15090017-15090873 |
| chr10: 26500608-26501126 | chr15: 101389733-101390260 | chr19: 15288315-15288911 |
| chr10: 26680635-26680999 | chr15: 101548676-101548898 | chr19: 15334123-15334822 |
| chr10: 28030183-28035211 | chr15: 23207950-23208796 | chr19: 15619224-15619613 |
| chr10: 29698363-29699044 | chr15: 24123232-24123432 | chr19: 15662114-15662358 |
| chr10: 31422710-31423316 | chr15: 28982670-28983497 | chr19: 15695299-15695535 |
| chr10: 35103125-35105243 | chr15: 30113706-30113940 | chr19: 16186790-16188275 |
| chr10: 43571940-43573434 | chr15: 31733082-31733489 | chr19: 1675922-1676137 |
| chr10: 44185040-44186132 | chr15: 32828333-32829181 | chr19: 17007102-17008857 |
| chr10: 45470023-45470291 | chr15: 34728788-34729495 | chr19: 1725416-1726154 |
| chr10: 45914375-45914883 | chr15: 37179517-37179782 | chr19: 17346166-17346886 |
| chr10: 49812853-49813352 | chr15: 37393667-37394248 | chr19: 17392384-17393775 |
| chr10: 51572199-51572620 | chr15: 40728263-40728466 | chr19: 17403040-17403570 |
| chr10: 52177725-52178182 | chr15: 41851724-41851970 | chr19: 17487896-17488125 |
| chr10: 57390344-57391215 | chr15: 41913334-41914360 | chr19: 17501956-17502250 |
| chr10: 60272468-60273456 | chr15: 41952536-41953222 | chr19: 17580198-17580598 |
| chr10: 60935828-60937049 | chr15: 45427297-45428206 | chr19: 1762373-1762848 |
| chr10: 62492786-62493376 | chr15: 45722562-45722939 | chr19: 1766145-1766364 |
| chr10: 62703308-62704290 | chr15: 48009419-48011044 | chr19: 17716645-17717531 |
| chr10: 64133790-64134420 | chr15: 48470008-48470628 | chr19: 1776028-1776730 |
| chr10: 71077947-71079377 | chr15: 51633725-51634318 | chr19: 17791068-17791301 |
| chr10: 71812319-71813693 | chr15: 52587354-52588172 | chr19: 17905580-17906317 |
| chr10: 71892093-71892802 | chr15: 60690709-60690930 | chr19: 18059850-18060148 |
| chr10: 72043447-72043886 | chr15: 61519622-61520031 | chr19: 18209912-18210268 |
| chr10: 72218131-72218484 | chr15: 61520424-61521716 | chr19: 18319146-18319363 |
| chr10: 72647739-72648317 | chr15: 62456016-62457648 | chr19: 18499098-18499697 |
| chr10: 72977124-72977774 | chr15: 64443588-64444821 | chr19: 18539782-18540341 |
| chr10: 734708-735606 | chr15: 65066917-65068352 | chr19: 1862257-1862600 |
| chr10: 74020774-74021023 | chr15: 65127965-65128185 | chr19: 18722359-18724001 |
| chr10: 74079337-74079861 | chr15: 65669311-65670617 | chr19: 18811562-18811791 |
| chr10: 75670697-75671379 | chr15: 65714743-65715571 | chr19: 19280999-19281560 |
| chr10: 77191062-77191571 | chr15: 66274584-66274838 | chr19: 19335772-19336151 |
| chr10: 79396096-79398495 | chr15: 66544433-66546134 | chr19: 19729128-19729814 |
| chr10: 8091375-8098329 | chr15: 68126023-68126569 | chr19: 19738573-19739821 |
| chr10: 81892223-81892640 | chr15: 68723368-68724691 | chr19: 19843483-19843943 |
| chr10: 85954200-85955303 | chr15: 69366322-69366796 | chr19: 19971578-19971863 |
| chr10: 86300400-86300953 | chr15: 71145996-71146820 | chr19: 20011911-20012238 |
| chr10: 88730555-88731632 | chr15: 72476677-72476969 | chr19: 20149856-20150175 |
| chr10: 90342529-90343221 | chr15: 72564443-72565165 | chr19: 20162773-20163107 |
| chr10: 93647053-93647393 | chr15: 72611947-72612802 | chr19: 2041824-2042593 |
| chr10: 94351357-94351615 | chr15: 74044562-74045885 | chr19: 21949904-21950217 |
| chr10: 94459255-94459591 | chr15: 74425092-74428821 | chr19: 22018595-22018827 |
| chr10: 94831513-94832312 | chr15: 74537810-74538114 | chr19: 22034395-22034943 |
| chr10: 95326230-95327108 | chr15: 75470912-75471212 | chr19: 22193331-22193583 |
| chr10: 95360390-95361387 | chr15: 75628251-75628677 | chr19: 2273694-2274047 |
| chr10: 95753348-95754201 | chr15: 78633323-78634089 | chr19: 2302467-2302999 |
| chr10: 97802872-97804262 | chr15: 79102716-79104628 | chr19: 23253797-23254382 |
| chr10: 98479726-98480487 | chr15: 83348840-83349865 | chr19: 23386650-23387069 |
| chr10: 99473085-99473291 | chr15: 83620951-83621727 | chr19: 23456575-23456868 |
| chr11: 100998031-100999774 | chr15: 83775862-83776922 | chr19: 2478866-2479244 |
| chr11: 101918074-101918493 | chr15: 83875649-83877079 | chr19: 2613933-2614133 |
| chr11: 102188180-102188440 | chr15: 84047823-84048105 | chr19: 267086-267660 |
| chr11: 106888324-106890203 | chr15: 84115763-84116921 | chr19: 28284529-28285129 |
| chr11: 109963241-109964677 | chr15: 84322323-84323771 | chr19: 295115-295354 |
| chr11: 112832525-112834490 | chr15: 84976071-84977044 | chr19: 30215233-30215594 |
| chr11: 113185333-113185663 | chr15: 89438177-89438850 | chr19: 30363203-30363527 |
| chr11: 113258444-113258821 | chr15: 89942593-89943853 | chr19: 31839636-31843049 |
| chr11: 113345069-113346328 | chr15: 89959743-89959990 | chr19: 31843881-31844183 |
| chr11: 113929634-113932190 | chr15: 90039465-90039984 | chr19: 31847947-31848209 |
| chr11: 113953621-113953839 | chr15: 91427541-91427905 | chr19: 3275609-3275970 |
| chr11: 116371183-116371606 | chr15: 93198375-93199181 | chr19: 33685147-33686085 |
| chr11: 116706482-116706910 | chr15: 93615623-93617185 | chr19: 34112280-34114353 |
| chr11: 119227097-119227758 | chr15: 96883942-96884533 | chr19: 3434930-3435417 |
| chr11: 119455154-119456102 | chr15: 96888959-96889417 | chr19: 34396472-34398079 |
| chr11: 119612092-119612476 | chr15: 99091356-99091812 | chr19: 34850072-34850587 |
| chr11: 120039602-120040210 | chr15: 99557979-99559170 | chr19: 35531781-35531991 |
| chr11: 120110498-120110719 | chr16: 10479687-10480254 | chr19: 36164078-36164286 |
| chr11: 120856726-120857174 | chr16: 1098523-1098875 | chr19: 36351441-36352167 |
| chr11: 121322539-121323302 | chr16: 11327009-11327234 | chr19: 3671903-3672121 |
| chr11: 122854931-122855582 | chr16: 1202415-1204624 | chr19: 3687787-3688262 |
| chr11: 123301050-123302149 | chr16: 12897366-12897889 | chr19: 36909282-36909854 |
| chr11: 124709223-124709957 | chr16: 1392545-1392753 | chr19: 37063893-37064749 |
| chr11: 124713006-124713395 | chr16: 1429051-1430104 | chr19: 37095681-37096589 |
| chr11: 124932731-124933657 | chr16: 1458144-1458435 | chr19: 37157633-37158119 |
| chr11: 126225356-126226073 | chr16: 19421817-19422498 | chr19: 3721465-3721788 |
| chr11: 126286452-126286870 | chr16: 203754-204866 | chr19: 37263382-37264083 |
| chr11: 12695415-12696981 | chr16: 2054012-2054288 | chr19: 37328897-37329518 |
| chr11: 128391713-128392611 | chr16: 2069719-2070865 | chr19: 37340919-37341262 |
| chr11: 129243999-129244567 | chr16: 2077043-2077470 | chr19: 37341732-37342000 |
| chr11: 129245109-129246395 | chr16: 21294981-21295414 | chr19: 37568953-37569549 |
| chr11: 129685738-129686211 | chr16: 21831483-21831944 | chr19: 37803517-37803822 |
| chr11: 130029659-130030262 | chr16: 2245867-2247100 | chr19: 37957727-37958390 |
| chr11: 130297402-130298517 | chr16: 25078048-25078532 | chr19: 37959853-37960615 |
| chr11: 133825338-133827457 | chr16: 2835064-2835958 | chr19: 38145827-38147219 |
| chr11: 134201785-134202407 | chr16: 28634441-28635031 | chr19: 38182794-38183327 |
| chr11: 13689589-13690724 | chr16: 28752083-28752332 | chr19: 38210108-38210692 |
| chr11: 15094958-15095872 | chr16: 29624388-29625391 | chr19: 3821975-3822278 |
| chr11: 1592499-1592810 | chr16: 29887789-29888316 | chr19: 38754847-38755699 |
| chr11: 16632509-16632725 | chr16: 30428871-30429799 | chr19: 38852312-38853485 |
| chr11: 17373020-17373665 | chr16: 3078142-3078553 | chr19: 39359917-39361160 |
| chr11: 17565767-17566052 | chr16: 3079537-3079954 | chr19: 39440405-39441011 |
| chr11: 18067717-18067928 | chr16: 30913356-30913942 | chr19: 39574701-39575137 |
| chr11: 18415923-18416680 | chr16: 31008833-31009036 | chr19: 39687598-39687964 |
| chr11: 18727321-18727727 | chr16: 31117102-31117435 | chr19: 39810993-39811293 |
| chr11: 19798538-19798949 | chr16: 31213567-31214287 | chr19: 39894559-39894768 |
| chr11: 20385162-20385673 | chr16: 31342453-31343140 | chr19: 39993358-39993765 |
| chr11: 2165136-2165672 | chr16: 31483277-31483646 | chr19: 39997591-39998291 |
| chr11: 22362863-22363377 | chr16: 31580071-31580317 | chr19: 40323908-40325005 |
| chr11: 22850766-22851367 | chr16: 31580560-31581023 | chr19: 40732076-40732665 |
| chr11: 2290105-2292932 | chr16: 3199653-3199937 | chr19: 41025315-41026106 |
| chr11: 2465172-2465648 | chr16: 3225355-3225594 | chr19: 41055125-41055331 |
| chr11: 2466248-2466818 | chr16: 32822902-32823585 | chr19: 41060059-41060565 |
| chr11: 27740645-27741236 | chr16: 3355021-3356012 | chr19: 41106059-41106415 |
| chr11: 30040073-30040294 | chr16: 4166656-4167012 | chr19: 41109614-41110075 |
| chr11: 30606005-30608128 | chr16: 4357553-4357955 | chr19: 41111084-41111767 |
| chr11: 3181575-3182120 | chr16: 4363936-4364151 | chr19: 41115446-41115767 |
| chr11: 32354761-32355484 | chr16: 4377509-4378162 | chr19: 41119032-41120394 |
| chr11: 32448262-32449744 | chr16: 4421643-4422240 | chr19: 41633777-41634047 |
| chr11: 35547140-35547396 | chr16: 4587837-4588890 | chr19: 41698744-41699325 |
| chr11: 416420-417650 | chr16: 46823933-46824263 | chr19: 42419339-42419564 |
| chr11: 43596991-43597336 | chr16: 47176788-47178446 | chr19: 42501262-42501486 |
| chr11: 43902255-43902528 | chr16: 48399731-48400040 | chr19: 42720827-42722132 |
| chr11: 45307442-45307975 | chr16: 50581476-50582735 | chr19: 42901017-42901375 |
| chr11: 45686161-45687495 | chr16: 52579637-52580845 | chr19: 42905724-42906350 |
| chr11: 45921388-45922167 | chr16: 52581294-52581610 | chr19: 42927286-42928598 |
| chr11: 46316876-46317485 | chr16: 54316904-54317149 | chr19: 43979360-43979684 |
| chr11: 46389075-46389378 | chr16: 54962423-54967805 | chr19: 44324437-44325009 |
| chr11: 47736740-47737106 | chr16: 55357522-55359040 | chr19: 44405818-44406053 |
| chr11: 49229718-49230040 | chr16: 56224732-56224980 | chr19: 44763979-44764312 |
| chr11: 504190-504659 | chr16: 56225256-56226351 | chr19: 44952417-44952809 |
| chr11: 57117316-57117575 | chr16: 56677121-56677598 | chr19: 45257704-45257918 |
| chr11: 57243682-57244463 | chr16: 56701864-56702208 | chr19: 45281134-45281355 |
| chr11: 57249947-57250848 | chr16: 56703388-56703648 | chr19: 4535070-4535339 |
| chr11: 58940831-58941052 | chr16: 57835956-57836989 | chr19: 4558364-4558579 |
| chr11: 61061752-61063063 | chr16: 58058713-58058928 | chr19: 45655294-45657246 |
| chr11: 61159837-61160285 | chr16: 58535041-58535596 | chr19: 45843871-45844418 |
| chr11: 61283865-61284147 | chr16: 66638255-66639561 | chr19: 45888769-45889465 |
| chr11: 61322733-61323285 | chr16: 66982435-66982945 | chr19: 45947711-45948047 |
| chr11: 61335113-61335340 | chr16: 67034471-67035340 | chr19: 46032305-46032855 |
| chr11: 61355039-61355363 | chr16: 67188614-67189652 | chr19: 4607080-4607350 |
| chr11: 61519742-61519972 | chr16: 67193006-67193219 | chr19: 46144873-46145631 |
| chr11: 61722745-61723483 | chr16: 67196793-67199988 | chr19: 46282545-46283127 |
| chr11: 62476771-62477481 | chr16: 67204209-67204637 | chr19: 46379884-46380207 |
| chr11: 62690975-62691488 | chr16: 67211934-67212509 | chr19: 46456210-46456503 |
| chr11: 62693374-62694772 | chr16: 67312897-67313731 | chr19: 46518284-46520080 |
| chr11: 63685490-63685968 | chr16: 67564059-67564332 | chr19: 46974558-46975073 |
| chr11: 63775347-63775613 | chr16: 67700010-67701354 | chr19: 46996328-46998437 |
| chr11: 63803376-63804365 | chr16: 68002838-68003039 | chr19: 47137781-47138070 |
| chr11: 64038965-64039306 | chr16: 68273546-68274084 | chr19: 47614409-47614661 |
| chr11: 64409878-64411253 | chr16: 68678687-68680149 | chr19: 47910108-47910563 |
| chr11: 64993281-64993484 | chr16: 69760294-69760506 | chr19: 47922252-47922777 |
| chr19: 48216487-48216837 | chr22: 23522551-23524465 | chr6: 10410110-10410690 |
| chr19: 48833395-48833720 | chr22: 23908323-23909178 | chr6: 10414217-10414498 |
| chr19: 48837142-48837549 | chr22: 24110030-24110995 | chr6: 10415019-10415318 |
| chr19: 48901805-48902123 | chr22: 24551814-24552696 | chr6: 10417385-10417842 |
| chr19: 49061546-49061769 | chr22: 26565200-26565986 | chr6: 10419400-10420323 |
| chr19: 49199965-49200184 | chr22: 27152698-27153133 | chr6: 10421415-10421727 |
| chr19: 49242019-49242962 | chr22: 29426511-29426843 | chr6: 10422322-10422648 |
| chr19: 49255779-49256495 | chr22: 29467292-29467817 | chr6: 10426201-10426638 |
| chr19: 49340489-49340774 | chr22: 31218227-31218893 | chr6: 105627407-105627830 |
| chr19: 49522774-49523093 | chr22: 32026298-32026874 | chr6: 106958233-106958718 |
| chr19: 49575130-49576076 | chr22: 33453893-33454505 | chr6: 106959765-106960985 |
| chr19: 49646093-49646308 | chr22: 37655606-37655839 | chr6: 108436110-108436522 |
| chr19: 49655103-49655395 | chr22: 37730552-37731415 | chr6: 108455061-108455273 |
| chr19: 49935752-49936275 | chr22: 38073038-38073412 | chr6: 112575092-112575483 |
| chr19: 49939611-49940045 | chr22: 38453188-38453503 | chr6: 117584585-117584816 |
| chr19: 50016532-50016851 | chr22: 38808785-38809017 | chr6: 118241348-118241569 |
| chr19: 50030982-50031300 | chr22: 39745984-39746634 | chr6: 124124178-124125309 |
| chr19: 50037079-50037670 | chr22: 39853324-39854065 | chr6: 125283125-125284389 |
| chr19: 50096610-50096912 | chr22: 40057942-40058844 | chr6: 125420744-125421141 |
| chr19: 50183923-50184172 | chr22: 40390485-40391490 | chr6: 127836731-127837706 |
| chr19: 50312012-50312821 | chr22: 42062791-42063002 | chr6: 1311069-1314220 |
| chr19: 50651174-50651535 | chr22: 42470036-42470669 | chr6: 132271357-132271658 |
| chr19: 50706483-50707196 | chr22: 43505952-43506167 | chr6: 134159127-134159349 |
| chr19: 50708602-50708855 | chr22: 45015934-45016488 | chr6: 134213960-134214351 |
| chr19: 50831455-50832070 | chr22: 45403037-45406372 | chr6: 13873766-13874262 |
| chr19: 50833814-50834128 | chr22: 46262066-46263747 | chr6: 13925100-13925510 |
| chr19: 50836391-50837447 | chr22: 46366727-46368726 | chr6: 146136326-146136564 |
| chr19: 51069360-51069600 | chr22: 50328694-50329178 | chr6: 150246753-150247257 |
| chr19: 51161924-51162514 | chr22: 50453057-50454137 | chr6: 150284683-150286515 |
| chr19: 51198809-51199347 | chr22: 50628950-50629249 | chr6: 150463772-150465002 |
| chr19: 51227662-51228883 | chr22: 50982571-50982994 | chr6: 151561284-151562550 |
| chr19: 51321268-51321781 | chr3: 101497831-101498648 | chr6: 151646669-151646958 |
| chr19: 51416003-51416249 | chr3: 101568108-101569046 | chr6: 152128823-152129771 |
| chr19: 51522005-51522803 | chr3: 10206450-10207024 | chr6: 152623280-152623480 |
| chr19: 51568155-51568412 | chr3: 112930438-112931506 | chr6: 152957811-152958472 |
| chr19: 51842129-51842353 | chr3: 113251700-113252290 | chr6: 155316300-155317197 |
| chr19: 52206245-52206726 | chr3: 117716101-117716564 | chr6: 158957221-158958677 |
| chr19: 52207133-52207731 | chr3: 119041383-119042255 | chr6: 159290674-159291195 |
| chr19: 52222424-52223208 | chr3: 119421856-119422334 | chr6: 1604607-1615866 |
| chr19: 52452317-52452543 | chr3: 120003884-120004426 | chr6: 166267510-166268461 |
| chr19: 52531324-52531724 | chr3: 120626881-120627579 | chr6: 166401527-166402659 |
| chr19: 52800400-52800871 | chr3: 122640714-122641355 | chr6: 17102310-17102539 |
| chr19: 52839445-52839937 | chr3: 12328995-12329227 | chr6: 19837505-19839314 |
| chr19: 52900956-52901158 | chr3: 124303530-124304092 | chr6: 21594614-21596437 |
| chr19: 5293011-5294211 | chr3: 124931162-124931747 | chr6: 21664508-21665178 |
| chr19: 53030825-53031359 | chr3: 126242784-126243358 | chr6: 21665716-21666031 |
| chr19: 53073309-53074039 | chr3: 127391012-127392434 | chr6: 21666656-21666862 |
| chr19: 53104385-53105149 | chr3: 128145258-128145656 | chr6: 24360030-24360411 |
| chr19: 53141176-53141813 | chr3: 128205496-128212274 | chr6: 25652381-25652709 |
| chr19: 53193140-53193945 | chr3: 128336407-128337113 | chr6: 26020672-26021125 |
| chr19: 5339641-5341061 | chr3: 128564772-128565007 | chr6: 26021958-26022193 |
| chr19: 53400461-53401205 | chr3: 129323916-129325332 | chr6: 26044204-26044469 |
| chr19: 53426079-53426389 | chr3: 129345668-129346521 | chr6: 26045645-26046033 |
| chr19: 53496733-53497028 | chr3: 129721369-129722131 | chr6: 26172034-26172398 |
| chr19: 53606060-53606784 | chr3: 13008649-13009210 | chr6: 26184037-26184336 |
| chr19: 53635626-53636230 | chr3: 131753881-131754139 | chr6: 26197071-26197537 |
| chr19: 53661431-53661848 | chr3: 133464950-133465420 | chr6: 26199198-26199466 |
| chr19: 53662190-53662500 | chr3: 134082861-134083201 | chr6: 26204765-26205213 |
| chr19: 53696030-53696650 | chr3: 134125488-134125871 | chr6: 26217212-26217521 |
| chr19: 54023869-54024560 | chr3: 136751342-136751683 | chr6: 26240698-26240951 |
| chr19: 54024646-54024923 | chr3: 138153270-138154621 | chr6: 26250437-26250827 |
| chr19: 54369388-54369809 | chr3: 140769887-140771080 | chr6: 26251899-26252233 |
| chr19: 54382665-54382951 | chr3: 145878431-145879287 | chr6: 26271346-26271588 |
| chr19: 54409967-54410200 | chr3: 145968335-145969008 | chr6: 26272471-26272696 |
| chr19: 54599161-54599456 | chr3: 150802997-150805168 | chr6: 26273291-26273557 |
| chr19: 54665922-54666377 | chr3: 152552603-152553712 | chr6: 26987599-26988074 |
| chr19: 54982389-54982897 | chr3: 156533840-156535131 | chr6: 27100175-27101124 |
| chr19: 5536532-5536789 | chr3: 157155268-157155826 | chr6: 27107139-27107394 |
| chr19: 55591434-55591644 | chr3: 158288801-158289271 | chr6: 27598688-27599146 |
| chr19: 55591906-55593980 | chr3: 159756633-159756997 | chr6: 27777856-27778251 |
| chr19: 55667533-55668465 | chr3: 160822495-160823260 | chr6: 27782248-27782485 |
| chr19: 55672024-55673212 | chr3: 168864031-168864449 | chr6: 27791781-27792217 |
| chr19: 5567519-5567939 | chr3: 169482339-169483052 | chr6: 27798991-27799427 |
| chr19: 55865017-55865434 | chr3: 169487146-169487522 | chr6: 27835191-27835461 |
| chr19: 55865825-55866324 | chr3: 173113271-173113681 | chr6: 28367125-28367651 |
| chr19: 55953372-55954641 | chr3: 179168736-179169593 | chr6: 2841811-2842273 |
| chr19: 56159258-56159937 | chr3: 179754521-179755245 | chr6: 2875602-2875811 |
| chr19: 56728589-56729375 | chr3: 181413015-181414022 | chr6: 2971087-2971962 |
| chr19: 56879418-56879995 | chr3: 181430142-181431076 | chr6: 3231596-3232289 |
| chr19: 56904637-56905355 | chr3: 181437185-181437478 | chr6: 32632159-32633027 |
| chr19: 56915357-56915856 | chr3: 182896921-182897381 | chr6: 33539023-33539339 |
| chr19: 57049676-57050646 | chr3: 182971430-182972635 | chr6: 36098040-36098621 |
| chr19: 57078366-57079301 | chr3: 183542497-183543804 | chr6: 39196818-39197517 |
| chr19: 57106576-57106896 | chr3: 184319377-184319822 | chr6: 40554596-40555928 |
| chr19: 57149424-57149631 | chr3: 184320006-184320218 | chr6: 40995803-40996241 |
| chr19: 57276615-57276942 | chr3: 184321784-184322277 | chr6: 41339237-41340027 |
| chr19: 57702697-57703425 | chr3: 18485113-18487056 | chr6: 41410590-41411140 |
| chr19: 57831600-57832250 | chr3: 184971642-184972002 | chr6: 41438063-41438594 |
| chr19: 57862442-57863236 | chr3: 185000558-185000896 | chr6: 42109905-42110361 |
| chr19: 57874764-57875110 | chr3: 186490603-186490813 | chr6: 4282005-4282310 |
| chr19: 57999026-57999613 | chr3: 186648175-186649103 | chr6: 42928219-42928810 |
| chr19: 58011125-58011743 | chr3: 187455331-187455843 | chr6: 43021126-43021694 |
| chr19: 5802235-5802569 | chr3: 187456251-187456580 | chr6: 43044045-43045057 |
| chr19: 58038573-58039208 | chr3: 187457732-187457948 | chr6: 43142014-43142217 |
| chr19: 58070554-58071273 | chr3: 188665276-188665552 | chr6: 43237261-43237643 |
| chr19: 58111230-58111770 | chr3: 193720954-193721291 | chr6: 43970074-43970922 |
| chr19: 58125531-58125902 | chr3: 193776092-193776308 | chr6: 44119527-44119781 |
| chr19: 58175624-58176104 | chr3: 193858771-193859695 | chr6: 44187187-44187400 |
| chr19: 58220190-58220517 | chr3: 194117602-194118988 | chr6: 45387347-45388164 |
| chr19: 58280927-58281486 | chr3: 194207386-194208785 | chr6: 4775132-4777550 |
| chr19: 58446337-58446800 | chr3: 196255496-196256013 | chr6: 49518316-49519186 |
| chr19: 58458687-58459219 | chr3: 197281606-197283128 | chr6: 52441270-52441793 |
| chr19: 58609339-58609988 | chr3: 197391890-197392481 | chr6: 53212618-53214043 |
| chr19: 58661738-58662287 | chr3: 23244051-23245071 | chr6: 54711154-54712033 |
| chr19: 58666024-58666833 | chr3: 24535845-24537436 | chr6: 56407465-56407926 |
| chr19: 58739944-58740554 | chr3: 24870675-24871369 | chr6: 56818874-56820308 |
| chr19: 58858454-58859223 | chr3: 27410613-27411066 | chr6: 69345217-69345483 |
| chr19: 58951215-58952250 | chr3: 27756213-27756422 | chr6: 70576975-70577572 |
| chr19: 59025309-59025705 | chr3: 27756661-27756941 | chr6: 710835-711297 |
| chr19: 6199293-6199551 | chr3: 32021970-32023364 | chr6: 7141037-7141576 |
| chr19: 6463992-6464780 | chr3: 32443153-32443455 | chr6: 72129509-72130756 |
| chr19: 6530827-6531552 | chr3: 38035702-38036000 | chr6: 73972820-73973027 |
| chr19: 6740670-6741203 | chr3: 38040290-38040535 | chr6: 74019429-74020053 |
| chr19: 675672-675881 | chr3: 38080629-38081187 | chr6: 74024403-74025020 |
| chr19: 6767946-6768160 | chr3: 38179858-38180689 | chr6: 74161087-74162090 |
| chr19: 681346-681576 | chr3: 40428652-40429015 | chr6: 74233269-74233546 |
| chr19: 7953281-7953708 | chr3: 40657160-40657395 | chr6: 75794881-75795088 |
| chr19: 8273531-8273920 | chr3: 44690127-44690587 | chr6: 75914706-75916387 |
| chr19: 8274242-8275045 | chr3: 44726929-44727237 | chr6: 83073417-83075319 |
| chr19: 9473590-9474001 | chr3: 44754100-44754399 | chr6: 87647254-87647707 |
| chr19: 9896564-9896994 | chr3: 44770937-44771137 | chr6: 94126273-94129677 |
| chr2: 100937780-100939059 | chr3: 45187027-45187946 | chr6: 96463870-96464136 |
| chr2: 101033607-101034296 | chr3: 46734732-46735717 | chr6: 99279318-99283842 |
| chr2: 10260280-10260931 | chr3: 46742765-46743086 | chr7: 100091181-100091598 |
| chr2: 102758807-102759577 | chr3: 46887629-46887919 | chr7: 100167137-100168144 |
| chr2: 105488369-105489991 | chr3: 46940055-46940394 | chr7: 100201615-100203652 |
| chr2: 105760110-105761018 | chr3: 50275394-50275645 | chr7: 100224271-100224558 |
| chr2: 106886119-106886738 | chr3: 50377804-50378540 | chr7: 100230650-100231320 |
| chr2: 107502354-107504216 | chr3: 50402104-50402942 | chr7: 100434983-100435193 |
| chr2: 10861207-10862382 | chr3: 50604906-50605359 | chr7: 100845051-100845726 |
| chr2: 110370907-110373301 | chr3: 51989764-51990639 | chr7: 102157824-102158510 |
| chr2: 110518072-110518913 | chr3: 52279602-52280140 | chr7: 102329951-102330654 |
| chr2: 112656187-112656918 | chr3: 53190510-53190764 | chr7: 104624338-104624848 |
| chr2: 119981080-119981818 | chr3: 54155240-54157025 | chr7: 106684814-106685765 |
| chr2: 121101047-121101432 | chr3: 55515153-55515613 | chr7: 108095402-108097206 |
| chr2: 121101801-121104534 | chr3: 55517659-55517939 | chr7: 114562171-114563193 |
| chr2: 121104713-121104935 | chr3: 55519090-55519623 | chr7: 115850371-115851028 |
| chr2: 121199724-121199993 | chr3: 55520227-55522344 | chr7: 116139775-116140352 |
| chr2: 121200504-121200788 | chr3: 55522561-55522836 | chr7: 121513047-121513911 |
| chr2: 121499229-121499578 | chr3: 56501869-56502345 | chr7: 12610166-12610834 |
| chr2: 121624828-121625209 | chr3: 57198244-57199378 | chr7: 126891301-126894205 |
| chr2: 127643801-127644104 | chr3: 57994922-57995218 | chr7: 126987644-126988233 |
| chr2: 127729673-127729905 | chr3: 59035407-59035944 | chr7: 127880751-127881375 |
| chr2: 128173476-128173793 | chr3: 62304515-62304780 | chr7: 128509207-128510201 |
| chr2: 128180295-128181381 | chr3: 62859618-62861190 | chr7: 128530630-128531356 |
| chr2: 130691703-130692102 | chr3: 64430586-64431192 | chr7: 128809090-128809393 |
| chr2: 131129719-131130511 | chr3: 65534685-65535344 | chr7: 128828334-128829366 |
| chr2: 131513364-131514183 | chr3: 69129059-69129515 | chr7: 130126018-130126801 |
| chr2: 131720820-131721867 | chr3: 69434927-69435583 | chr7: 130417913-130419378 |
| chr2: 131792238-131793189 | chr3: 69591012-69592050 | chr7: 132260475-132262527 |
| chr2: 132121264-132121762 | chr3: 72788073-72788416 | chr7: 1329382-1329673 |
| chr2: 132285409-132285992 | chr3: 73673231-73674375 | chr7: 133812006-133812233 |
| chr2: 133173533-133174369 | chr3: 74663507-74664121 | chr7: 138666359-138666972 |
| chr2: 133426653-133428870 | chr3: 75834255-75834761 | chr7: 138720045-138721019 |
| chr2: 134023948-134024467 | chr3: 77088499-77089198 | chr7: 139167481-139168712 |
| chr2: 134785626-134785845 | chr3: 85008285-85008837 | chr7: 14029379-14029593 |
| chr2: 145273457-145275378 | chr3: 96532024-96533625 | chr7: 1408517-1408878 |
| chr2: 145281737-145282269 | chr3: 98451287-98452225 | chr7: 148036495-148036848 |
| chr2: 14772378-14775809 | chr3: 98620132-98620910 | chr7: 148982080-148982675 |
| chr2: 154334450-154335458 | chr3: 9904332-9904811 | chr7: 149119399-149120307 |
| chr2: 155554006-155556069 | chr3: 9956823-9957664 | chr7: 149495-150038 |
| chr2: 160654230-160654631 | chr3: 99594970-99595215 | chr7: 150019951-150020752 |
| chr2: 160918675-160919177 | chr3: 9993731-9994136 | chr7: 150655109-150655643 |
| chr2: 161263872-161264460 | chr4: 1005931-1006282 | chr7: 150675207-150675866 |
| chr2: 162930234-162930879 | chr4: 102711830-102712199 | chr7: 150676179-150676585 |
| chr2: 164592918-164593511 | chr4: 103997209-103998007 | chr7: 151078463-151079536 |
| chr2: 166649910-166650966 | chr4: 10462833-10463689 | chr7: 151136934-151137273 |
| chr2: 168149034-168150797 | chr4: 1049653-1049923 | chr7: 154861798-154862074 |
| chr2: 171569878-171573904 | chr4: 109683730-109684362 | chr7: 155579869-155580392 |
| chr2: 172944989-172945384 | chr4: 111532648-111533391 | chr7: 156810573-156814593 |
| chr2: 172945912-172946212 | chr4: 113435899-113438002 | chr7: 158785262-158785771 |
| chr2: 172947844-172948200 | chr4: 113445023-113445350 | chr7: 16460779-16461265 |
| chr2: 172949243-172950126 | chr4: 1161128-1161682 | chr7: 1703862-1710517 |
| chr2: 172951290-172952159 | chr4: 118006539-118006859 | chr7: 191876-193799 |
| chr2: 172952810-172953160 | chr4: 119273306-119274458 | chr7: 20817456-20818227 |
| chr2: 172959268-172962054 | chr4: 123747741-123748980 | chr7: 20823308-20825662 |
| chr2: 172971046-172971387 | chr4: 124333-124841 | chr7: 21582444-21583313 |
| chr2: 173600282-173600970 | chr4: 124426781-124427157 | chr7: 21985072-21985842 |
| chr2: 1746834-1748971 | chr4: 126235896-126238930 | chr7: 22893795-22894577 |
| chr2: 174877566-174877778 | chr4: 13529255-13529865 | chr7: 23513535-23514412 |
| chr2: 175193399-175193764 | chr4: 13548571-13549956 | chr7: 24323559-24325080 |
| chr2: 175199464-175202639 | chr4: 1396292-1401730 | chr7: 25891957-25892615 |
| chr2: 175204844-175207553 | chr4: 141294667-141295274 | chr7: 25898238-25898771 |
| chr2: 176989338-176989587 | chr4: 142053328-142054601 | chr7: 26191795-26192757 |
| chr2: 176993480-176995557 | chr4: 154143933-154144463 | chr7: 27190275-27191115 |
| chr2: 177001222-177001783 | chr4: 154170369-154170600 | chr7: 27194584-27194827 |
| chr2: 177005365-177005666 | chr4: 154605087-154606052 | chr7: 27208872-27209616 |
| chr2: 177029414-177029941 | chr4: 156588298-156589292 | chr7: 27212417-27214396 |
| chr2: 177052958-177054350 | chr4: 15704641-15705000 | chr7: 27219310-27219750 |
| chr2: 177502241-177502910 | chr4: 157892686-157893286 | chr7: 27224268-27224596 |
| chr2: 180725718-180726465 | chr4: 157997167-157997686 | chr7: 27275056-27275626 |
| chr2: 183902403-183903625 | chr4: 166299976-166300668 | chr7: 29603110-29603328 |
| chr2: 185463209-185463589 | chr4: 166794546-166795341 | chr7: 30028519-30029822 |
| chr2: 189156417-189157719 | chr4: 168155006-168155765 | chr7: 31232678-31232900 |
| chr2: 191044980-191045829 | chr4: 170946767-170947925 | chr7: 35301151-35301731 |
| chr2: 192109829-192110940 | chr4: 171010944-171011503 | chr7: 37487355-37488672 |
| chr2: 197457393-197458522 | chr4: 175750331-175750584 | chr7: 37960317-37961046 |
| chr2: 200320503-200329681 | chr4: 176922474-176922820 | chr7: 38670440-38671008 |
| chr2: 201450527-201451027 | chr4: 176923076-176923630 | chr7: 42276004-42277850 |
| chr2: 201983199-201983709 | chr4: 176986922-176987360 | chr7: 42533077-42533283 |
| chr2: 207308267-207308907 | chr4: 183369277-183370459 | chr7: 44079718-44080568 |
| chr2: 20865290-20867589 | chr4: 184319565-184320198 | chr7: 44364433-44365328 |
| chr2: 21022565-21022934 | chr4: 184643915-184644414 | chr7: 48128047-48128499 |
| chr2: 213402181-213403867 | chr4: 186455984-186456864 | chr7: 5111621-5112088 |
| chr2: 214148958-214149426 | chr4: 187476280-187476844 | chr7: 51383312-51384938 |
| chr2: 217236026-217237129 | chr4: 190942735-190944898 | chr7: 5336514-5336894 |
| chr2: 217497812-217498847 | chr4: 21950086-21950346 | chr7: 54731780-54732479 |
| chr2: 217556803-217557174 | chr4: 26030421-26030692 | chr7: 55000877-55001135 |
| chr2: 218621280-218621498 | chr4: 2765639-2766063 | chr7: 56183578-56184165 |
| chr2: 219156978-219157380 | chr4: 298804-299312 | chr7: 63767751-63767991 |
| chr2: 219252422-219252631 | chr4: 30718854-30719847 | chr7: 64349425-64350151 |
| chr2: 219748277-219748805 | chr4: 30721205-30724842 | chr7: 64407590-64408299 |
| chr2: 219762988-219763537 | chr4: 330163-332068 | chr7: 65037625-65037864 |
| chr2: 219773547-219774103 | chr4: 3464800-3465440 | chr7: 65509075-65509594 |
| chr2: 219827660-219828205 | chr4: 379900-380399 | chr7: 65878464-65878977 |
| chr2: 219866132-219868054 | chr4: 38869402-38869780 | chr7: 6654746-6655860 |
| chr2: 219922155-219923130 | chr4: 4108735-4109499 | chr7: 69062375-69065037 |
| chr2: 219924820-219926130 | chr4: 41218315-41218768 | chr7: 70060835-70061456 |
| chr2: 220159439-220159977 | chr4: 41646293-41646582 | chr7: 73021487-73021720 |
| chr2: 220196432-220196768 | chr4: 41753670-41753947 | chr7: 73867788-73868061 |
| chr2: 220348956-220349784 | chr4: 42399153-42400802 | chr7: 74202334-74203629 |
| chr2: 220361463-220363254 | chr4: 4577005-4577448 | chr7: 74572475-74573770 |
| chr2: 220377744-220377946 | chr4: 46391904-46392572 | chr7: 75889087-75889345 |
| chr2: 220406364-220406840 | chr4: 48271363-48271846 | chr7: 75889498-75889760 |
| chr2: 223288923-223290013 | chr4: 48272027-48272298 | chr7: 75896511-75896944 |
| chr2: 223536123-223536565 | chr4: 48492118-48493589 | chr7: 76026791-76027223 |
| chr2: 224701963-224702790 | chr4: 4854614-4855210 | chr7: 77649015-77649543 |
| chr2: 225906654-225907464 | chr4: 4873263-4873613 | chr7: 82072022-82073520 |
| chr2: 228582486-228582821 | chr4: 48908293-48908850 | chr7: 82791675-82792412 |
| chr2: 228736231-228736544 | chr4: 52917389-52918280 | chr7: 8473140-8475199 |
| chr2: 229045958-229046553 | chr4: 53617119-53617607 | chr7: 84814840-84816242 |
| chr2: 230135709-230136004 | chr4: 55991403-55992171 | chr7: 852961-853230 |
| chr2: 231692698-231693496 | chr4: 5713036-5713451 | chr7: 87256959-87258444 |
| chr2: 231712776-231712982 | chr4: 57687611-57687906 | chr7: 88388489-88389283 |
| chr2: 231902027-231903122 | chr4: 62065884-62068801 | chr7: 90225083-90226364 |
| chr2: 232276743-232277135 | chr4: 667274-667601 | chr7: 915754-916644 |
| chr2: 232765247-232765491 | chr4: 7194538-7195467 | chr7: 94536850-94537477 |
| chr2: 232791576-232792016 | chr4: 74486045-74486258 | chr7: 95025560-95026122 |
| chr2: 233367781-233368577 | chr4: 74702421-74702627 | chr7: 96631384-96631800 |
| chr2: 233740669-233741879 | chr4: 74809870-74810089 | chr7: 96746768-96747190 |
| chr2: 234776883-234777098 | chr4: 74864114-74864329 | chr7: 98971650-98973170 |
| chr2: 236578081-236580153 | chr4: 74964657-74965279 | chr7: 993402-994574 |
| chr2: 238395062-238396241 | chr4: 77507078-77507339 | chr7: 99768885-99769559 |
| chr2: 238599858-238601430 | chr4: 77610325-77610943 | chr8: 102092795-102093240 |
| chr2: 238768074-238768831 | chr4: 786119-786341 | chr8: 103822615-103823263 |
| chr2: 239755097-239758310 | chr4: 7940564-7941853 | chr8: 104310810-104311620 |
| chr2: 241391877-241393598 | chr4: 79472807-79473177 | chr8: 104383410-104384109 |
| chr2: 241395249-241395802 | chr4: 81123509-81124318 | chr8: 104510871-104513913 |
| chr2: 241771656-241772194 | chr4: 81124469-81124845 | chr8: 105235385-105235979 |
| chr2: 241855375-241855631 | chr4: 81951942-81952808 | chr8: 105600454-105601704 |
| chr2: 241922778-241923143 | chr4: 8594413-8594777 | chr8: 10586614-10586886 |
| chr2: 242498014-242499274 | chr4: 87515184-87515735 | chr8: 107669778-107670591 |
| chr2: 242742599-242743758 | chr4: 8869045-8870067 | chr8: 110374553-110374793 |
| chr2: 242785912-242786616 | chr4: 8893372-8896268 | chr8: 11058625-11058844 |
| chr2: 242808406-242808618 | chr4: 89378224-89378948 | chr8: 11059012-11059242 |
| chr2: 242823447-242823970 | chr4: 91048412-91049682 | chr8: 110986114-110986983 |
| chr2: 24300061-24300294 | chr4: 95678799-95679809 | chr8: 11555100-11555603 |
| chr2: 26396103-26397204 | chr5: 102201584-102201912 | chr8: 11759863-11760212 |
| chr2: 26401696-26402099 | chr5: 10307521-10307913 | chr8: 119963947-119964178 |
| chr2: 26407423-26408253 | chr5: 112630220-112630624 | chr8: 119964390-119964637 |
| chr2: 26726190-26726710 | chr5: 112823257-112824304 | chr8: 120220389-120221222 |
| chr2: 26785115-26785687 | chr5: 113391072-113392005 | chr8: 12990091-12990914 |
| chr2: 27341568-27341923 | chr5: 113696517-113699195 | chr8: 13133843-13134312 |
| chr2: 27958208-27958723 | chr5: 115151349-115152713 | chr8: 131455249-131456285 |
| chr2: 31360322-31361821 | chr5: 11903551-11904703 | chr8: 133492399-133493586 |
| chr2: 31456741-31457653 | chr5: 121412501-121414077 | chr8: 142318354-142319155 |
| chr2: 38763115-38763564 | chr5: 121647451-121648147 | chr8: 143530646-143530872 |
| chr2: 42068364-42068594 | chr5: 122424906-122425958 | chr8: 143531119-143534495 |
| chr2: 43864323-43864956 | chr5: 122426126-122426836 | chr8: 143556715-143556997 |
| chr2: 45161428-45163058 | chr5: 122433879-122435551 | chr8: 144099457-144099731 |
| chr2: 45235512-45237792 | chr5: 125930602-125931049 | chr8: 144503421-144503808 |
| chr2: 47499465-47499813 | chr5: 126564998-126565580 | chr8: 144640397-144640772 |
| chr2: 47796924-47799166 | chr5: 127872564-127874945 | chr8: 144798488-144799038 |
| chr2: 48757212-48757785 | chr5: 128300801-128301329 | chr8: 144822012-144822805 |
| chr2: 54785027-54785969 | chr5: 128795504-128797417 | chr8: 144842965-144843542 |
| chr2: 56410866-56412539 | chr5: 131346893-131347776 | chr8: 145555343-145562310 |
| chr2: 58273643-58274422 | chr5: 132155289-132155497 | chr8: 145697880-145698784 |
| chr2: 64836207-64836765 | chr5: 132946745-132948467 | chr8: 145700239-145700822 |
| chr2: 65086805-65087180 | chr5: 134824932-134825224 | chr8: 145806259-145806713 |
| chr2: 66660453-66660794 | chr5: 134827286-134827644 | chr8: 145909677-145912846 |
| chr2: 66661119-66661657 | chr5: 135170272-135171156 | chr8: 15397637-15398287 |
| chr2: 68870352-68871055 | chr5: 135527057-135528978 | chr8: 16884364-16885339 |
| chr2: 69240084-69241004 | chr5: 139017134-139017668 | chr8: 17354322-17355250 |
| chr2: 70351316-70351550 | chr5: 139040820-139041028 | chr8: 1771362-1772760 |
| chr2: 7057531-7058433 | chr5: 139047906-139048235 | chr8: 22089368-22089668 |
| chr2: 70994448-70995385 | chr5: 139076543-139077179 | chr8: 22408673-22409635 |
| chr2: 71192095-71192495 | chr5: 139081002-139081202 | chr8: 22456092-22456508 |
| chr2: 71503548-71504233 | chr5: 139135590-139135976 | chr8: 22457124-22457753 |
| chr2: 71680559-71681276 | chr5: 139174905-139176172 | chr8: 22960385-22960927 |
| chr2: 71693165-71694127 | chr5: 139227606-139228279 | chr8: 23081957-23082975 |
| chr2: 73151201-73152060 | chr5: 139742900-139743148 | chr8: 23260443-23261867 |
| chr2: 74347274-74347777 | chr5: 139927158-139927491 | chr8: 24770909-24772547 |
| chr2: 74425445-74426423 | chr5: 140011483-140012739 | chr8: 25897201-25897612 |
| chr2: 74725040-74727038 | chr5: 140167092-140168139 | chr8: 26371173-26372830 |
| chr2: 74776017-74776897 | chr5: 140207726-140208078 | chr8: 27183091-27183390 |
| chr2: 74781495-74782685 | chr5: 140261885-140262153 | chr8: 27348659-27348883 |
| chr2: 75147006-75147336 | chr5: 140531158-140532017 | chr8: 29210484-29210801 |
| chr2: 75426593-75428030 | chr5: 140568217-140569118 | chr8: 33371898-33372526 |
| chr2: 7571072-7571642 | chr5: 140573424-140574316 | chr8: 35092680-35093559 |
| chr2: 75787718-75788312 | chr5: 140604454-140605304 | chr8: 35093901-35094111 |
| chr2: 79220193-79220592 | chr5: 140615392-140616353 | chr8: 38325092-38326374 |
| chr2: 79739697-79740243 | chr5: 140620865-140621698 | chr8: 38644474-38645760 |
| chr2: 85359957-85362593 | chr5: 140741175-140741738 | chr8: 40755165-40755404 |
| chr2: 85640970-85641259 | chr5: 140750051-140750264 | chr8: 41624556-41625380 |
| chr2: 85811341-85811855 | chr5: 140762402-140762768 | chr8: 41654876-41655984 |
| chr2: 85980500-85982198 | chr5: 140767197-140767695 | chr8: 49231523-49231810 |
| chr2: 8818293-8818503 | chr5: 140777443-140777938 | chr8: 494156-496083 |
| chr2: 88316234-88316859 | chr5: 140802400-140802832 | chr8: 49647703-49647988 |
| chr2: 88469692-88470386 | chr5: 141132812-141133338 | chr8: 54569668-54570010 |
| chr2: 96054895-96055143 | chr5: 141228931-141230084 | chr8: 54789582-54790665 |
| chr2: 96192056-96193072 | chr5: 141705392-141705688 | chr8: 54791846-54795141 |
| chr2: 96314996-96315230 | chr5: 142782072-142785071 | chr8: 57232371-57232795 |
| chr2: 98703355-98703889 | chr5: 145316146-145316354 | chr8: 58907002-58907822 |
| chr20: 1206680-1207119 | chr5: 146257347-146258575 | chr8: 60030135-60032356 |
| chr20: 13200671-13202616 | chr5: 146614284-146614560 | chr8: 61193313-61194195 |
| chr20: 13975769-13976287 | chr5: 149681703-149682601 | chr8: 65491084-65491319 |
| chr20: 13976701-13977068 | chr5: 150004671-150004984 | chr8: 65492936-65494452 |
| chr20: 18039533-18039928 | chr5: 150284386-150284635 | chr8: 6691834-6693135 |
| chr20: 1874934-1875718 | chr5: 150325905-150326194 | chr8: 67089250-67089962 |
| chr20: 19738040-19739773 | chr5: 150537020-150537418 | chr8: 6949350-6950039 |
| chr20: 22562737-22566104 | chr5: 151304227-151304824 | chr8: 70946891-70947299 |
| chr20: 23028404-23032218 | chr5: 153853057-153853551 | chr8: 72917306-72917641 |
| chr20: 24898955-24899181 | chr5: 153862143-153862451 | chr8: 74005022-74005856 |
| chr20: 30196119-30196585 | chr5: 154026819-154027257 | chr8: 7543874-7544385 |
| chr20: 30582789-30583099 | chr5: 15500077-15500923 | chr8: 80695831-80696458 |
| chr20: 30605745-30605970 | chr5: 155107505-155108934 | chr8: 81805956-81806327 |
| chr20: 3229239-3229693 | chr5: 157002175-157003182 | chr8: 82192379-82193685 |
| chr20: 32450501-32450765 | chr5: 157285770-157286254 | chr8: 82644604-82644849 |
| chr20: 33865768-33866091 | chr5: 158758475-158758839 | chr8: 8820767-8821112 |
| chr20: 3387966-3388901 | chr5: 159343216-159343574 | chr8: 89339966-89340481 |
| chr20: 34188720-34190267 | chr5: 159738782-159739913 | chr8: 95651207-95651760 |
| chr20: 36793550-36793867 | chr5: 160974693-160975384 | chr8: 95652456-95652873 |
| chr20: 37230524-37230742 | chr5: 16179065-16180420 | chr8: 95653899-95654733 |
| chr20: 37274692-37275134 | chr5: 168727430-168728275 | chr8: 97156764-97158030 |
| chr20: 39311379-39312435 | chr5: 170288880-170289737 | chr8: 97505748-97507607 |
| chr20: 39316551-39319987 | chr5: 170877799-170878210 | chr8: 9756178-9756993 |
| chr20: 4202149-4202765 | chr5: 172068287-172069174 | chr8: 99305904-99306726 |
| chr20: 42285962-42286535 | chr5: 172385523-172385912 | chr9: 103173890-103174153 |
| chr20: 43438738-43439546 | chr5: 172710766-172711062 | chr9: 103790613-103791764 |
| chr20: 44098281-44099536 | chr5: 172754057-172757098 | chr9: 103791945-103792173 |
| chr20: 44452577-44453162 | chr5: 174151479-174152364 | chr9: 104248248-104249501 |
| chr20: 44539730-44540099 | chr5: 175223610-175224679 | chr9: 1051820-1052240 |
| chr20: 44642095-44642406 | chr5: 175621334-175621827 | chr9: 10612636-10613333 |
| chr20: 44746823-44747060 | chr5: 176236762-176238081 | chr9: 107509907-107510768 |
| chr20: 44935933-44937310 | chr5: 176789979-176790296 | chr9: 110249749-110252660 |
| chr20: 45142001-45142337 | chr5: 176830276-176831639 | chr9: 112262011-112262317 |
| chr20: 45523251-45524020 | chr5: 177098635-177099525 | chr9: 112402768-112403349 |
| chr20: 47443735-47445181 | chr5: 177366539-177366973 | chr9: 114287381-114287695 |
| chr20: 48598960-48599657 | chr5: 177433282-177434067 | chr9: 116111664-116112189 |
| chr20: 50158905-50159509 | chr5: 177540208-177541234 | chr9: 116450146-116450454 |
| chr20: 55500348-55501102 | chr5: 178016559-178017670 | chr9: 116860474-116860695 |
| chr20: 55839288-55839766 | chr5: 178322714-178323538 | chr9: 123631107-123631672 |
| chr20: 55840217-55841794 | chr5: 178367621-178368725 | chr9: 123690772-123691675 |
| chr20: 55964273-55964656 | chr5: 178770725-178772794 | chr9: 124061806-124062229 |
| chr20: 55964917-55965271 | chr5: 180479586-180480959 | chr9: 124461798-124462190 |
| chr20: 56323974-56324254 | chr5: 180542154-180542402 | chr9: 124498514-124498962 |
| chr20: 56725858-56726113 | chr5: 2038528-2038949 | chr9: 124975754-124976692 |
| chr20: 57224696-57226322 | chr5: 31855004-31855426 | chr9: 125109008-125109644 |
| chr20: 57581903-57582595 | chr5: 36690208-36690658 | chr9: 126135408-126136193 |
| chr20: 57797224-57797441 | chr5: 373843-374426 | chr9: 126762469-126762683 |
| chr20: 59826978-59828978 | chr5: 38556223-38557563 | chr9: 126807511-126808181 |
| chr20: 6103437-6103970 | chr5: 38845503-38846476 | chr9: 129677707-129678009 |
| chr20: 61147458-61147787 | chr5: 41510325-41510651 | chr9: 130461544-130461839 |
| chr20: 61200973-61201272 | chr5: 42423531-42423740 | chr9: 131012455-131013429 |
| chr20: 61456340-61456565 | chr5: 42424339-42425047 | chr9: 131965038-131965636 |
| chr20: 61884645-61886387 | chr5: 42994627-42994936 | chr9: 132020630-132021038 |
| chr20: 61927195-61927482 | chr5: 42995123-42995415 | chr9: 132082872-132083582 |
| chr20: 61937483-61937738 | chr5: 43017969-43018668 | chr9: 132099124-132099616 |
| chr20: 61992187-61993599 | chr5: 43040346-43040633 | chr9: 132145577-132146328 |
| chr20: 62600654-62601676 | chr5: 43040846-43041161 | chr9: 132331219-132331458 |
| chr20: 62673793-62674131 | chr5: 43396898-43397364 | chr9: 132359673-132360061 |
| chr20: 62714764-62715761 | chr5: 472601-474261 | chr9: 132382433-132383004 |
| chr20: 62958974-62959513 | chr5: 474959-475319 | chr9: 132499969-132500553 |
| chr20: 708602-709290 | chr5: 49736608-49737300 | chr9: 13278313-13279805 |
| chr20: 8112885-8113592 | chr5: 55776605-55777233 | chr9: 132934214-132934483 |
| chr20: 9048959-9050018 | chr5: 57878726-57879177 | chr9: 133308594-133309448 |
| chr20: 9819272-9819861 | chr5: 58334837-58335881 | chr9: 133412891-133413096 |
| chr21: 18984536-18985697 | chr5: 60921535-60922472 | chr9: 134151854-134153015 |
| chr21: 27011625-27012398 | chr5: 6448754-6449629 | chr9: 134158161-134158682 |
| chr21: 28216559-28218117 | chr5: 66299769-66300083 | chr9: 136451013-136451276 |
| chr21: 32929928-32932017 | chr5: 67584214-67584451 | chr9: 137217063-137218078 |
| chr21: 36041306-36043224 | chr5: 68710808-68711520 | chr9: 137299191-137299437 |
| chr21: 38119794-38120742 | chr5: 691081-691376 | chr9: 137533360-137534397 |
| chr21: 38352857-38353274 | chr5: 72415612-72416766 | chr9: 138985838-138987846 |
| chr21: 38362016-38362868 | chr5: 72715408-72715997 | chr9: 139014622-139014848 |
| chr21: 40032244-40033665 | chr5: 72732366-72733732 | chr9: 139159210-139159560 |
| chr21: 40760627-40760829 | chr5: 74349801-74350239 | chr9: 139551255-139551559 |
| chr21: 42878752-42880674 | chr5: 75378975-75380796 | chr9: 139552948-139553269 |
| chr21: 43373136-43374062 | chr5: 76011121-76012292 | chr9: 139553660-139553915 |
| chr21: 43917047-43917268 | chr5: 76115511-76116089 | chr9: 139595846-139596130 |
| chr21: 44073202-44074650 | chr5: 76941396-76941888 | chr9: 139872238-139873143 |
| chr21: 45148455-45149262 | chr5: 78365299-78365711 | chr9: 140051063-140051730 |
| chr21: 46129392-46129689 | chr5: 87437096-87437505 | chr9: 140317161-140318663 |
| chr21: 46351329-46352911 | chr5: 87976095-87976546 | chr9: 14348685-14349074 |
| chr21: 46706692-46707049 | chr5: 92906240-92908875 | chr9: 14349308-14349515 |
| chr22: 17849475-17850733 | chr5: 94619460-94621121 | chr9: 17134822-17135706 |
| chr22: 18923471-18923840 | chr5: 95170618-95170855 | chr9: 214587-215431 |
| chr22: 19753313-19755013 | chr5: 9544693-9546715 | chr9: 21559134-21559816 |
| chr22: 21319179-21319912 | chr5: 96038210-96038884 | chr9: 2241892-2242102 |
| chr22: 22862624-22863220 | chr6: 101841426-101841905 | chr9: 27528358-27528725 |
| chr9: 27528977-27529885 | | |
| chr9: 33044246-33044612 | | |
| chr9: 33447447-33447824 | | |
| chr9: 33750520-33751160 | | |
| chr9: 34377402-34377610 | | |
| chr9: 34379542-34380017 | | |
| chr9: 34577867-34578258 | | |
| chr9: 34589114-34591978 | | |
| chr9: 35756949-35757339 | | |
| chr9: 36036799-36037564 | | |
| chr9: 36258171-36258886 | | |
| chr9: 37575919-37576445 | | |
| chr9: 38069785-38069991 | | |
| chr9: 38423948-38424584 | | |
| chr9: 4297818-4300182 | | |
| chr9: 46148701-46149726 | | |
| chr9: 4662253-4662951 | | |
| chr9: 707022-707420 | | |
| chr9: 71788716-71789542 | | |
| chr9: 72658837-72659277 | | |
| chr9: 77502094-77502518 | | |
| chr9: 79073908-79074561 | | |
| chr9: 79520804-79521508 | | |
| chr9: 80911780-80912611 | | |
| chr9: 85677016-85678321 | | |
| chr9: 86571048-86572027 | | |
| chr9: 8857486-8858708 | | |
| chr9: 88713706-88714908 | | |
| chr9: 89560585-89562647 | | |
| chr9: 90112515-90113817 | | |
| chr9: 90340716-90341542 | | |
| chr9: 90589210-90589807 | | |
| chr9: 93563776-93564546 | | |
| chr9: 93955501-93956420 | | |
| chr9: 94183408-94183994 | | |
| chr9: 95569430-95572255 | | |
| chr9: 95896008-95897016 | | |
| chr9: 97021465-97021967 | | |
| chr9: 97766650-97767955 | | |
| chr9: 97810766-97811272 | | |
| chr9: 99145525-99145849 | | |

TABLE 4
Additional Example CGIs

| | | |
|---|---|---|
| chr1: 10762450-10766925 | chr12: 101107864-101113622 | chr17: 48039283-48045064 |
| chr1: 110608266-110615303 | chr12: 103694091-103698418 | chr17: 48192635-48197085 |
| chr1: 113263574-113267787 | chr12: 104695349-104699984 | chr17: 48543571-48548900 |
| chr1: 113284333-113289172 | chr12: 106972413-106983086 | chr17: 4998370-5003205 |
| chr1: 114693137-114698672 | chr12: 113011100-113015529 | chr17: 50233176-50238466 |
| chr1: 115878168-115883332 | chr12: 113513165-113517970 | chr17: 59483574-59487780 |
| chr1: 116378360-116384364 | chr12: 113588807-113593304 | chr17: 59526980-59537254 |
| chr1: 1179757-1184470 | chr12: 113898751-113918717 | chr17: 6614423-6619471 |
| chr1: 119524783-119532712 | chr12: 114831912-114854360 | chr17: 6677206-6681710 |
| chr1: 119541057-119553320 | chr12: 114876144-114888579 | chr17: 70109980-70122442 |
| chr1: 12121489-12126148 | chr12: 115107504-115112061 | chr17: 71946479-71951255 |
| chr1: 145073484-145077845 | chr12: 117796077-117801448 | chr17: 72853622-72860012 |
| chr1: 146550329-146554577 | chr12: 119210111-119214393 | chr17: 72913569-72918510 |
| chr1: 1468605-1477220 | chr12: 120833587-120837927 | chr17: 73747619-73752178 |
| chr1: 147780067-147784473 | chr12: 122014171-122019693 | chr17: 74015770-74020658 |
| chr1: 149330994-149335389 | chr12: 123752050-123756373 | chr17: 74531282-74536566 |
| chr1: 155145186-155149444 | chr12: 127208779-127213651 | chr17: 75240872-75254180 |
| chr1: 155262319-155267536 | chr12: 127938452-127942907 | chr17: 75275318-75280172 |
| chr1: 155288607-155293001 | chr12: 129335871-129340653 | chr17: 75366689-75372506 |
| chr1: 156103708-156108171 | chr12: 130385610-130391139 | chr17: 75396285-75400527 |
| chr1: 156336759-156341251 | chr12: 130906778-130911191 | chr17: 75445478-75449821 |
| chr1: 156356051-156360252 | chr12: 131197825-131202157 | chr17: 77803867-77811046 |
| chr1: 156388404-156393581 | chr12: 132903450-132908206 | chr17: 7830533-7835164 |
| chr1: 156861416-156865711 | chr12: 14132627-14137242 | chr17: 78997641-79001641 |
| chr1: 160338605-160342843 | chr12: 15473319-15477901 | chr17: 7903928-7909445 |
| chr1: 161693638-161699298 | chr12: 184864-189610 | chr17: 79312963-79322653 |
| chr1: 164543541-164547917 | chr12: 29300035-29304954 | chr17: 79857809-79862963 |
| chr1: 165321704-165328328 | chr12: 3306813-3312270 | chr17: 932418-937088 |
| chr1: 16858874-16864296 | chr12: 3473011-3477654 | chr18: 11146308-11151936 |
| chr1: 170628457-170632851 | chr12: 41084523-41089102 | chr18: 11748954-11754756 |
| chr1: 173636663-173641045 | chr12: 45442203-45447386 | chr18: 12252148-12257089 |
| chr1: 175566377-175570808 | chr12: 48397169-48401372 | chr18: 13639585-13644415 |
| chr1: 177131393-177135846 | chr12: 49181050-49185282 | chr18: 13866533-13871026 |
| chr1: 179542721-179547307 | chr12: 49369691-49377550 | chr18: 19742937-19754363 |
| chr1: 180196120-180206975 | chr12: 49482921-49487178 | chr18: 30347691-30354302 |
| chr1: 181285301-181289873 | chr12: 5016586-5023171 | chr18: 35142908-35149628 |
| chr1: 181450707-181455073 | chr12: 5151013-5156346 | chr18: 43606141-43610510 |
| chr1: 18434552-18439673 | chr12: 52113411-52117679 | chr18: 44334184-44340100 |
| chr1: 18954896-18970739 | chr12: 52406382-52410675 | chr18: 44770993-44780084 |
| chr1: 19201875-19206234 | chr12: 52650019-52654743 | chr18: 44787407-44792678 |
| chr1: 197885089-197889791 | chr12: 53105913-53110471 | chr18: 54786960-54791194 |
| chr1: 200007808-200012036 | chr12: 53357193-53361507 | chr18: 55017708-55023605 |
| chr1: 201250453-201255648 | chr12: 53489573-53493955 | chr18: 55092826-55110853 |
| chr1: 202160959-202165390 | chr12: 54069054-54073265 | chr18: 55920988-55926068 |
| chr1: 202676882-202681769 | chr12: 54319302-54323721 | chr18: 56885092-56889665 |
| chr1: 203042723-203047390 | chr12: 54336762-54341168 | chr18: 56937625-56943540 |
| chr1: 208130328-208135117 | chr12: 54352530-54382102 | chr18: 58998684-59003692 |
| chr1: 214151215-214161080 | chr12: 54421428-54428709 | chr18: 61141927-61145927 |
| chr1: 21614381-21619101 | chr12: 54438643-54450091 | chr18: 70531966-70538871 |
| chr1: 217308750-217313178 | chr12: 54517769-54522457 | chr18: 72914108-72919233 |
| chr1: 221048449-221070185 | chr12: 57616770-57621402 | chr18: 73165403-73169920 |
| chr1: 225863069-225867328 | chr12: 58001881-58006249 | chr18: 74151240-74157073 |
| chr1: 226073151-226077680 | chr12: 58156856-58162000 | chr18: 74797145-74802038 |
| chr1: 226125113-226129695 | chr12: 63541637-63546967 | chr18: 74959557-74965822 |
| chr1: 228783987-228788204 | chr12: 6436273-6440931 | chr18: 76730971-76743244 |
| chr1: 231294560-231299345 | chr12: 65216246-65221143 | chr18: 77545966-77560948 |
| chr1: 24227116-24231537 | chr12: 65512879-65517863 | chr18: 902579-911574 |
| chr1: 243644395-243648888 | chr12: 72663684-72669551 | chr19: 10404935-10409342 |
| chr1: 248018331-248023252 | chr12: 75600992-75605344 | chr19: 10461627-10466378 |
| chr1: 25253528-25261005 | chr12: 81100035-81104716 | chr19: 1061545-1066265 |
| chr1: 2770127-2774665 | chr12: 81469570-81474119 | chr19: 1106395-1111610 |
| chr1: 29583898-29588598 | chr12: 99137387-99141769 | chr19: 11592373-11596987 |
| chr1: 2977276-2982758 | chr13: 100545634-100550911 | chr19: 12664244-12668682 |
| chr1: 32050472-32054771 | chr13: 100639335-100644188 | chr19: 12765750-12769980 |
| chr1: 34626784-34632976 | chr13: 102566426-102571495 | chr19: 12829794-12834225 |
| chr1: 34640383-34645024 | chr13: 108516335-108521063 | chr19: 12878575-12882888 |
| chr1: 36547555-36551965 | chr13: 109145799-109151019 | chr19: 13122960-13127259 |
| chr1: 38217703-38222012 | chr13: 112705805-112730419 | chr19: 13133318-13138169 |
| chr1: 38459585-38463988 | chr13: 112756599-112763113 | chr19: 13196700-13200999 |
| chr1: 38939920-38944404 | chr13: 20873519-20878214 | chr19: 13211451-13215821 |
| chr1: 39042060-39046561 | chr13: 27332227-27337205 | chr19: 13614753-13619267 |
| chr1: 39978366-39983768 | chr13: 28364550-28370505 | chr19: 14087571-14091796 |
| chr1: 40233768-40239190 | chr13: 28496227-28501046 | chr19: 15290400-15294632 |
| chr1: 40767187-40771871 | chr13: 28547840-28552246 | chr19: 1746168-1752243 |
| chr1: 41282848-41287149 | chr13: 32887117-32892116 | chr19: 18977352-18983200 |
| chr1: 41829977-41834542 | chr13: 36042845-36055119 | chr19: 19366709-19374393 |
| chr1: 44029287-44033853 | chr13: 51415372-51420149 | chr19: 21767190-21771786 |
| chr1: 46949169-46953792 | chr13: 53417898-53424872 | chr19: 2422006-2429983 |
| chr1: 47007576-47012132 | chr13: 58201587-58210930 | chr19: 30713550-30719970 |
| chr1: 4711990-4718555 | chr13: 79179945-79185880 | chr19: 33623468-33627805 |
| chr1: 47907713-47913020 | chr13: 84451665-84455897 | chr19: 35631410-35635697 |
| chr1: 50878917-50884103 | chr13: 93877246-93882877 | chr19: 36244329-36249982 |
| chr1: 50890438-50895243 | chr14: 101190852-101195499 | chr19: 36334276-36339138 |
| chr1: 53525573-53530974 | chr14: 101921576-101927995 | chr19: 36498170-36502530 |
| chr1: 53740298-53744845 | chr14: 103653242-103657928 | chr19: 36521392-36525887 |
| chr1: 55503061-55508015 | chr14: 105165664-105170129 | chr19: 3866587-3871217 |
| chr1: 61513876-61518831 | chr14: 24042887-24048760 | chr19: 38698334-38702577 |
| chr1: 63780395-63798140 | chr14: 24639054-24644220 | chr19: 38874071-38878332 |
| chr1: 65729412-65733849 | chr14: 24801679-24806353 | chr19: 39735690-39741288 |
| chr1: 65989002-65993811 | chr14: 29234836-29239832 | chr19: 39752974-39758540 |
| chr1: 66256441-66260918 | chr14: 29252366-29257069 | chr19: 40312927-40317144 |
| chr1: 67216080-67220293 | chr14: 33400095-33406079 | chr19: 405012-411511 |
| chr1: 67771330-67775767 | chr14: 36971170-36996488 | chr19: 42889312-42893646 |
| chr1: 77745315-77750224 | chr14: 37047334-37055690 | chr19: 44201559-44205987 |
| chr1: 86619279-86624871 | chr14: 37114189-37138348 | chr19: 44276274-44280777 |
| chr1: 91170103-91194804 | chr14: 38676246-38682937 | chr19: 45258353-45263809 |
| chr1: 91298980-91303891 | chr14: 38722255-38727537 | chr19: 45896880-45902315 |
| chr1: 92943908-92954609 | chr14: 48141434-48147589 | chr19: 45999831-46004686 |
| chr10: 100990157-100994687 | chr14: 51336713-51341146 | chr19: 46316491-46321266 |
| chr10: 101277942-101292338 | chr14: 52732208-52737486 | chr19: 46913312-46917802 |
| chr10: 102277163-102281730 | chr14: 54416678-54420881 | chr19: 47149769-47155125 |
| chr10: 102417148-102421668 | chr14: 57258879-57286558 | chr19: 48963003-48967792 |
| chr10: 102471207-102493011 | chr14: 58329677-58335121 | chr19: 49667276-49671552 |
| chr10: 102505483-102511646 | chr14: 60971773-60980180 | chr19: 50879419-50883664 |
| chr10: 102889011-102908693 | chr14: 61101979-61106663 | chr19: 50929271-50933638 |
| chr10: 102973970-102980096 | chr14: 62277477-62282019 | chr19: 51167660-51174023 |
| chr10: 102994035-102998646 | chr14: 69254677-69259036 | chr19: 51599823-51604260 |
| chr10: 103041991-103046480 | chr14: 74704189-74710192 | chr19: 51813158-51817458 |
| chr10: 105359785-105364188 | chr14: 77734734-77739772 | chr19: 54410711-54415087 |
| chr10: 105418686-105423076 | chr14: 85995469-86002478 | chr19: 54479413-54485572 |
| chr10: 105525044-105529044 | chr14: 92787495-92792712 | chr19: 55595978-55600887 |
| chr10: 106397568-106404812 | chr14: 95235623-95241679 | chr19: 55813941-55818277 |
| chr10: 108921781-108926805 | chr14: 95824676-95828941 | chr19: 56596039-56602296 |
| chr10: 109672197-109676964 | chr15: 100911439-100916022 | chr19: 56986314-56991741 |
| chr10: 110669725-110674326 | chr15: 23155795-23160624 | chr19: 58092740-58097764 |
| chr10: 111214605-111219083 | chr15: 27110031-27115479 | chr19: 5827049-5831474 |
| chr10: 118028733-118036230 | chr15: 27213952-27218856 | chr19: 58543116-58556587 |
| chr10: 118890162-118902329 | chr15: 33007531-33013696 | chr19: 7931264-7936898 |
| chr10: 118998436-119003530 | chr15: 33600817-33606003 | chr19: 8672333-8676764 |
| chr10: 119309205-119315563 | chr15: 35044444-35049480 | chr19: 868775-873318 |
| chr10: 119492494-119496991 | chr15: 37388176-37392380 | chr2: 102801673-102806556 |
| chr10: 120351693-120357821 | chr15: 40266582-40271061 | chr2: 105457128-105482760 |
| chr10: 121575530-121580385 | chr15: 45406468-45411528 | chr2: 106679983-106684403 |
| chr10: 123920851-123925542 | chr15: 47474370-47479499 | chr2: 107101834-107106053 |
| chr10: 124899908-124913035 | chr15: 49252985-49257564 | chr2: 108600825-108605467 |
| chr10: 125423496-125428642 | chr15: 53074188-53089488 | chr2: 114031360-114038041 |
| chr10: 125648821-125653373 | chr15: 53095562-53100476 | chr2: 114254776-114260043 |
| chr10: 125730221-125734843 | chr15: 59155046-59159594 | chr2: 118979770-118984466 |
| chr10: 129532411-129539366 | chr15: 60285108-60300520 | chr2: 119590603-119618826 |
| chr10: 130336696-130340994 | chr15: 67071307-67075943 | chr2: 119912127-119918663 |
| chr10: 130506444-130510658 | chr15: 74417871-74425044 | chr2: 124780253-124785255 |
| chr10: 131262948-131267947 | chr15: 76628030-76635515 | chr2: 127411697-127416171 |
| chr10: 134595358-134604649 | chr15: 79572831-79577211 | chr2: 127780614-127784829 |
| chr10: 15759424-15764101 | chr15: 79722100-79727643 | chr2: 128419720-128424182 |
| chr10: 16559605-16565822 | chr15: 89145661-89151198 | chr2: 130761484-130765764 |
| chr10: 1776785-1782018 | chr15: 89310720-89315183 | chr2: 132180328-132185101 |
| chr10: 22621351-22636862 | chr15: 89901447-89924768 | chr2: 137520461-137525696 |
| chr10: 22762709-22769050 | chr15: 89947374-89955182 | chr2: 139535693-139540650 |
| chr10: 23459301-23465889 | chr15: 91640909-91645702 | chr2: 142885725-142890553 |
| chr10: 23478698-23484455 | chr15: 96871409-96879721 | chr2: 144692667-144697180 |
| chr10: 23981367-23986978 | chr15: 96893307-96912030 | chr2: 154725907-154731328 |
| chr10: 26502384-26509434 | chr15: 96957342-96962531 | chr2: 157183558-157188355 |
| chr10: 27545669-27550402 | chr16: 10910160-10914719 | chr2: 162271295-162286677 |
| chr10: 43426168-43431460 | chr16: 20082708-20087305 | chr2: 171669599-171682358 |
| chr10: 48436412-48441320 | chr16: 2226191-2232946 | chr2: 176929576-176984402 |
| chr10: 50600990-50608783 | chr16: 22822617-22828459 | chr2: 177010372-177027692 |
| chr10: 50815602-50822356 | chr16: 23722271-23726775 | chr2: 177034255-177045444 |
| chr10: 63210496-63215009 | chr16: 24265041-24269527 | chr2: 182319762-182325029 |
| chr10: 71329450-71335392 | chr16: 3011017-3015228 | chr2: 182519222-182523927 |
| chr10: 75405414-75409706 | chr16: 3065522-3070358 | chr2: 19558964-19563650 |
| chr10: 76571196-76575507 | chr16: 31051480-31055800 | chr2: 198027069-198031438 |
| chr10: 8074003-8080378 | chr16: 3188766-3193389 | chr2: 200331688-200336172 |
| chr10: 88120925-88129364 | chr16: 3218439-3223356 | chr2: 207504775-207509422 |
| chr10: 94178316-94182754 | chr16: 48842552-48847264 | chr2: 20868007-20873280 |
| chr10: 94453525-94457896 | chr16: 49307124-49319263 | chr2: 219734133-219738788 |
| chr10: 94818027-94831040 | chr16: 49870450-49874926 | chr2: 219846292-219860917 |
| chr10: 99787615-99793320 | chr16: 51145491-51149944 | chr2: 220171871-220176283 |
| chr11: 105479127-105483422 | chr16: 51166267-51171110 | chr2: 220297484-220302243 |
| chr11: 115628399-115633117 | chr16: 51181700-51190763 | chr2: 220410342-220414678 |
| chr11: 119291321-119295943 | chr16: 54323041-54327703 | chr2: 223157726-223187468 |
| chr11: 123064518-123068986 | chr16: 54968302-54974846 | chr2: 233249362-233255414 |
| chr11: 124627724-124631926 | chr16: 55362824-55367483 | chr2: 237066072-237080762 |
| chr11: 128417199-128421513 | chr16: 55511221-55515526 | chr2: 238862316-238867170 |
| chr11: 128692085-128696688 | chr16: 58028215-58033633 | chr2: 241457633-241462047 |
| chr11: 131778329-131783532 | chr16: 6066915-6072401 | chr2: 241756142-241762783 |
| chr11: 132811563-132816395 | chr16: 62067122-62072634 | chr2: 25497764-25502429 |
| chr11: 132932060-132936291 | chr16: 66610750-66615412 | chr2: 30451567-30457655 |
| chr11: 132950539-132955307 | chr16: 67206068-67210678 | chr2: 31803294-31808403 |
| chr11: 133992710-133997090 | chr16: 67569253-67574728 | chr2: 3284325-3288530 |
| chr11: 14993129-14997908 | chr16: 67916680-67920909 | chr2: 3748829-3753927 |
| chr11: 17738790-17745779 | chr16: 68478865-68484822 | chr2: 38299277-38306518 |
| chr11: 20179201-20184325 | chr16: 71457782-71462338 | chr2: 45153196-45159049 |
| chr11: 20616198-20625399 | chr16: 82658652-82663813 | chr2: 45167506-45173884 |
| chr11: 27741473-27746564 | chr16: 84000270-84004860 | chr2: 45225645-45230783 |
| chr11: 2888389-2893337 | chr16: 86528748-86534994 | chr2: 45238373-45243579 |
| chr11: 31823744-31850776 | chr16: 86547070-86552512 | chr2: 45393870-45400186 |
| chr11: 32450145-32459311 | chr16: 86609389-86615821 | chr2: 465850-470659 |
| chr11: 36395927-36401398 | chr16: 88941428-88945669 | chr2: 50572046-50576817 |
| chr11: 43566922-43571854 | chr17: 1171536-1176733 | chr2: 54084777-54089266 |
| chr11: 44323658-44329932 | chr17: 12566668-12571335 | chr2: 5829188-5834208 |
| chr11: 46297545-46302216 | chr17: 12875271-12879773 | chr2: 63280515-63289097 |
| chr11: 46364877-46369101 | chr17: 14199727-14204052 | chr2: 66650692-66656218 |
| chr11: 60716429-60720888 | chr17: 14246392-14250721 | chr2: 66670432-66675636 |
| chr11: 61535001-61539001 | chr17: 15818621-15823325 | chr2: 66806569-66811404 |
| chr11: 624729-642628 | chr17: 1878790-1883116 | chr2: 71785431-71789897 |
| chr11: 64134815-64140187 | chr17: 19881326-19885610 | chr2: 73141056-73150260 |
| chr11: 64476844-64481598 | chr17: 21365115-21369592 | chr2: 80527678-80532846 |
| chr11: 64813041-64817722 | chr17: 27897512-27902067 | chr2: 80547579-80551798 |
| chr11: 65350232-65355134 | chr17: 32482008-32486280 | chr2: 87013975-87020182 |
| chr11: 65407637-65412127 | chr17: 33774554-33778888 | chr2: 87086817-87091037 |
| chr11: 65814405-65818665 | chr17: 35289900-35302875 | chr2: 97190978-97195383 |
| chr11: 67348566-67353565 | chr17: 36715692-36720593 | chr20: 10196136-10200984 |
| chr11: 68620109-68624339 | chr17: 37319483-37324099 | chr20: 17204529-17210756 |
| chr11: 69515841-69521929 | chr17: 37761692-37766304 | chr20: 21374359-21380245 |
| chr11: 69829572-69834484 | chr17: 40935259-40939480 | chr20: 21483933-21498714 |
| chr11: 70209532-70213532 | chr17: 41275001-41280000 | chr20: 21684200-21697344 |
| chr11: 70506329-70510617 | chr17: 43035167-43039740 | chr20: 22546968-22561240 |
| chr11: 70670835-70675055 | chr17: 43470528-43476343 | chr20: 25061839-25067525 |
| chr11: 71950113-71954528 | chr17: 45947677-45951885 | chr20: 2537134-2541877 |
| chr11: 723597-728870 | chr17: 46602363-46610390 | chr20: 2727998-2733630 |
| chr11: 72530613-72535774 | chr17: 46618368-46634212 | chr20: 2778979-2783497 |
| chr11: 79146359-79154200 | chr17: 46667435-46676181 | chr20: 3143122-3147746 |
| chr11: 8188227-8192671 | chr17: 46689521-46699701 | chr20: 32854660-32859248 |
| chr11: 88239711-88244562 | chr17: 46794235-46802746 | chr20: 33294515-33300242 |
| chr11: 89222417-89226718 | chr17: 46822786-46827372 | chr20: 36010596-36015439 |
| chr20: 36224618-36228841 | chr6: 161186085-161190639 | chr20: 44655464-44661243 |
| chr20: 37350131-37359372 | chr6: 1617094-1623094 | |
| chr20: 39992546-39997810 | chr6: 166577974-166585423 | |
| chr20: 41815476-41821212 | chr6: 166664838-166669541 | |
| chr20: 44683772-44689610 | chr6: 170730120-170734442 | |
| chr20: 48182194-48186833 | chr6: 26612014-26616851 | |
| chr20: 51587708-51592020 | chr6: 27226101-27230364 | |
| chr20: 52787253-52792986 | chr6: 29593299-29597795 | |
| chr20: 5294267-5299798 | chr6: 29892141-29897117 | |
| chr20: 57087461-57092237 | chr6: 30093174-30097610 | |
| chr20: 57413136-57429047 | chr6: 30137719-30142263 | |
| chr20: 61701527-61706022 | chr6: 33046417-33050814 | |
| chr20: 688576-693099 | chr6: 33391593-33395908 | |
| chr20: 9494472-9498893 | chr6: 33653967-33658238 | |
| chr21: 19615099-19619874 | chr6: 35477389-35481678 | |
| chr21: 31309387-31314106 | chr6: 37614723-37619179 | |
| chr21: 32622145-32626382 | chr6: 38680950-38685265 | |
| chr21: 34393129-34402245 | chr6: 389189-395790 | |
| chr21: 38063180-38068185 | chr6: 4077053-4081443 | |
| chr21: 38074763-38083833 | chr6: 41526267-41530900 | |
| chr21: 42216490-42221222 | chr6: 41906746-41911711 | |
| chr22: 19744156-19748369 | chr6: 42070033-42074701 | |
| chr22: 19965280-19969808 | chr6: 42143848-42148053 | |
| chr22: 25079851-25084112 | chr6: 42877280-42881623 | |
| chr22: 29707282-29714013 | chr6: 46653263-46658738 | |
| chr22: 31196492-31201033 | chr6: 50680335-50685214 | |
| chr22: 31498397-31503239 | chr6: 50785287-50793573 | |
| chr22: 37210770-37215467 | chr6: 50808643-50820431 | |
| chr22: 37463057-37467331 | chr6: 55037171-55041392 | |
| chr22: 37909980-37914258 | chr6: 5995028-6009797 | |
| chr22: 38377094-38381964 | chr6: 70990041-70994912 | |
| chr22: 38474837-38480839 | chr6: 7227878-7232865 | |
| chr22: 39260339-39265211 | chr6: 72296275-72300528 | |
| chr22: 42303618-42309254 | chr6: 78170232-78176088 | |
| chr22: 42320044-42324909 | chr6: 85470703-85476132 | |
| chr22: 42683895-42688095 | chr6: 99273764-99278038 | |
| chr22: 44255943-44260612 | chr6: 99288280-99292771 | |
| chr22: 44285498-44290061 | chr7: 100073304-100077551 | |
| chr22: 44724725-44729590 | chr7: 100815485-100825701 | |
| chr22: 46316694-46321087 | chr7: 101003900-101009443 | |
| chr22: 46438394-46443019 | chr7: 103083711-103088132 | |
| chr22: 48882885-48889043 | chr7: 103966784-103971959 | |
| chr22: 50494442-50499393 | chr7: 113722925-113729795 | |
| chr3: 11032447-11037384 | chr7: 12149221-12153559 | |
| chr3: 113158300-113162641 | chr7: 121938007-121959341 | |
| chr3: 121900743-121905645 | chr7: 124402175-124406432 | |
| chr3: 126111548-126115967 | chr7: 127988927-127994616 | |
| chr3: 127631994-127636588 | chr7: 128553330-128558650 | |
| chr3: 127792370-127798136 | chr7: 129418287-129425355 | |
| chr3: 12836472-12840782 | chr7: 130788359-130794773 | |
| chr3: 128717866-128723245 | chr7: 1360812-1365643 | |
| chr3: 129691128-129696841 | chr7: 136551855-136558194 | |
| chr3: 13112628-13117245 | chr7: 142492564-142497248 | |
| chr3: 133391119-133395657 | chr7: 143580126-143584610 | |
| chr3: 137480965-137493004 | chr7: 149387655-149391976 | |
| chr3: 138654628-138661107 | chr7: 149742403-149748469 | |
| chr3: 147106512-147116479 | chr7: 152619917-152624149 | |
| chr3: 147124989-147144391 | chr7: 153746408-153752444 | |
| chr3: 154144348-154148965 | chr7: 153999965-154004281 | |
| chr3: 157810054-157823836 | chr7: 155162558-155177248 | |
| chr3: 170301045-170305768 | chr7: 155239324-155245757 | |
| chr3: 172163373-172168738 | chr7: 155256828-155263403 | |
| chr3: 184054420-184058671 | chr7: 155300254-155305158 | |
| chr3: 185909345-185914228 | chr7: 155593693-155607095 | |
| chr3: 186076711-186082111 | chr7: 156407024-156411865 | |
| chr3: 19187689-19192100 | chr7: 156793356-156803632 | |
| chr3: 192123822-192129994 | chr7: 156869055-156873297 | |
| chr3: 22411493-22416365 | chr7: 158934508-158940492 | |
| chr3: 236392-242140 | chr7: 19143873-19148256 | |
| chr3: 26662105-26666796 | chr7: 19182819-19187033 | |
| chr3: 27769639-27773942 | chr7: 20368004-20373504 | |
| chr3: 32859142-32863429 | chr7: 20828568-20832817 | |
| chr3: 3838514-3844772 | chr7: 23285222-23289508 | |
| chr3: 44061315-44065837 | chr7: 26413747-26418891 | |
| chr3: 44594536-44599018 | chr7: 27132098-27136736 | |
| chr3: 46616308-46620669 | chr7: 27144070-27150389 | |
| chr3: 49945622-49950430 | chr7: 27180614-27187562 | |
| chr3: 55506337-55510708 | chr7: 27195602-27208462 | |
| chr3: 62352292-62365082 | chr7: 27225521-27231043 | |
| chr3: 63261990-63266205 | chr7: 27258102-27262467 | |
| chr3: 64251534-64255819 | chr7: 27276946-27294197 | |
| chr3: 6900824-6906641 | chr7: 30719373-30724445 | |
| chr3: 71832069-71836653 | chr7: 32108064-32112910 | |
| chr3: 75665778-75671067 | chr7: 35294922-35300218 | |
| chr3: 75953760-75958308 | chr7: 37953623-37958555 | |
| chr3: 87839797-87844563 | chr7: 42265547-42269823 | |
| chr3: 9175692-9180189 | chr7: 43150021-43155340 | |
| chr4: 100868378-100873994 | chr7: 49811009-49817752 | |
| chr4: 105147-109898 | chr7: 53284852-53289192 | |
| chr4: 107954556-107959453 | chr7: 54610325-54614558 | |
| chr4: 109091039-109096546 | chr7: 56353509-56357798 | |
| chr4: 110220971-110226257 | chr7: 6588564-6592957 | |
| chr4: 111552966-111557504 | chr7: 6659876-6664695 | |
| chr4: 114898356-114902810 | chr7: 70594229-70600382 | |
| chr4: 122299568-122304290 | chr7: 71798758-71804768 | |
| chr4: 128542032-128546903 | chr7: 72836384-72840815 | |
| chr4: 134067163-134072442 | chr7: 73892816-73897110 | |
| chr4: 13522063-13528083 | chr7: 749713-754150 | |
| chr4: 140199065-140203449 | chr7: 87561343-87566571 | |
| chr4: 144618823-144624218 | chr7: 89745893-89751036 | |
| chr4: 147557206-147563901 | chr7: 90891568-90898683 | |
| chr4: 151502012-151507085 | chr7: 95223504-95228194 | |
| chr4: 154707513-154716240 | chr7: 96648222-96654246 | |
| chr4: 155661810-155666315 | chr7: 97359133-97365018 | |
| chr4: 156127169-156132209 | chr7: 97839637-97844005 | |
| chr4: 156678096-156683386 | chr8: 101115923-101120693 | |
| chr4: 15777999-15782729 | chr8: 102502479-102506841 | |
| chr4: 158141297-158146053 | chr8: 105476673-105481340 | |
| chr4: 164262822-164267772 | chr8: 11534768-11540961 | |
| chr4: 169797087-169801625 | chr8: 11555853-11569212 | |
| chr4: 172731735-172737118 | chr8: 120426399-120431178 | |
| chr4: 174420025-174462054 | chr8: 130993922-130998149 | |
| chr4: 185935243-185944747 | chr8: 132050204-132056749 | |
| chr4: 187217321-187221745 | chr8: 139506796-139511774 | |
| chr4: 188914606-188918876 | chr8: 142526186-142531029 | |
| chr4: 190935926-190942591 | chr8: 143543446-143548178 | |
| chr4: 204378-208892 | chr8: 144806222-144812978 | |
| chr4: 24799110-24803902 | chr8: 144988271-145004135 | |
| chr4: 25088107-25092510 | chr8: 145101286-145110027 | |
| chr4: 41747185-41751811 | chr8: 145923411-145928101 | |
| chr4: 41867175-41884964 | chr8: 21642909-21649845 | |
| chr4: 46993129-46997872 | chr8: 21903462-21907757 | |
| chr4: 47032428-47036940 | chr8: 23560476-23569678 | |
| chr4: 4857633-4871173 | chr8: 24810947-24816299 | |
| chr4: 54964164-54978202 | chr8: 25898563-25907842 | |
| chr4: 5707986-5712495 | chr8: 26719643-26726566 | |
| chr4: 57519622-57524703 | chr8: 37820487-37826008 | |
| chr4: 5889204-5897116 | chr8: 41422342-41427300 | |
| chr4: 66533194-66537620 | chr8: 4846969-4854635 | |
| chr4: 680725-685079 | chr8: 49466684-49470959 | |
| chr4: 81107888-81121391 | chr8: 50820271-50824860 | |
| chr4: 85401831-85425190 | chr8: 53849702-53856426 | |
| chr4: 90226715-90231010 | chr8: 55364181-55382186 | |
| chr4: 93224349-93229007 | chr8: 57356127-57361415 | |
| chr4: 94753787-94758310 | chr8: 65279904-65292946 | |
| chr4: 959348-964155 | chr8: 65708991-65713722 | |
| chr5: 11382682-11387521 | chr8: 68862585-68866946 | |
| chr5: 115695135-115699589 | chr8: 70979874-70986888 | |
| chr5: 122428677-122433443 | chr8: 72466561-72471561 | |
| chr5: 134361093-134388370 | chr8: 85094760-85099247 | |
| chr5: 139136876-139141242 | chr8: 86348766-86353196 | |
| chr5: 140050060-140055381 | chr8: 87079654-87084046 | |
| chr5: 140303713-140309193 | chr8: 97167732-97174022 | |
| chr5: 140344106-140348931 | chr8: 9758751-9766748 | |
| chr5: 140785448-140790044 | chr8: 98287605-98292404 | |
| chr5: 140796758-140801359 | chr8: 99958498-99963438 | |
| chr5: 140808495-140814617 | chr8: 99982585-99988983 | |
| chr5: 140862528-140866748 | chr9: 100608697-100622192 | |
| chr5: 145716290-145727852 | chr9: 102588743-102593303 | |
| chr5: 146886751-146891840 | chr9: 104497850-104503076 | |
| chr5: 148031473-148036080 | chr9: 112079403-112084905 | |
| chr5: 158476379-158480630 | chr9: 115820072-115825416 | |
| chr5: 158521907-158526598 | chr9: 120173254-120179496 | |
| chr5: 159397005-159401928 | chr9: 120505228-120509642 | |
| chr5: 170733170-170746107 | chr9: 122129087-122134214 | |
| chr5: 172108283-172113166 | chr9: 123654751-123658972 | |
| chr5: 172657050-172674971 | chr9: 124411513-124416193 | |
| chr5: 174156681-174161729 | chr9: 124985744-124993086 | |
| chr5: 175083005-175087756 | chr9: 126771247-126782953 | |
| chr5: 178419226-178424337 | chr9: 129370738-129391231 | |
| chr5: 179226284-179231003 | chr9: 131152347-131157923 | |
| chr5: 180484155-180488892 | chr9: 132457588-132462017 | |
| chr5: 1872908-1889743 | chr9: 133532535-133544394 | |
| chr5: 2736954-2759024 | chr9: 134427867-134432491 | |
| chr5: 31191953-31196419 | chr9: 135036714-135041978 | |
| chr5: 3588645-3605054 | chr9: 135453165-135468240 | |
| chr5: 37832672-37837128 | chr9: 136292739-136297236 | |
| chr5: 38255826-38261136 | chr9: 137965111-137969727 | |
| chr5: 45693395-45698510 | chr9: 139094666-139098993 | |
| chr5: 50683454-50688148 | chr9: 139394206-139399040 | |
| chr5: 52775789-52779996 | chr9: 139713664-139718441 | |
| chr5: 54517055-54529760 | chr9: 16724860-16729273 | |
| chr5: 59187047-59191894 | chr9: 17904420-17909488 | |
| chr5: 63253045-63259886 | chr9: 19786216-19791288 | |
| chr5: 71012918-71017715 | chr9: 21968914-21973190 | |
| chr5: 72524204-72531976 | chr9: 22003888-22008229 | |
| chr5: 72592148-72597808 | chr9: 23818692-23824135 | |
| chr5: 72674121-72680421 | chr9: 23848911-23853522 | |
| chr5: 76921888-76938984 | chr9: 32780937-32785625 | |
| chr5: 77138543-77149785 | chr9: 35842850-35846850 | |
| chr5: 77251833-77256049 | chr9: 36737535-36741782 | |
| chr5: 77266351-77270787 | chr9: 37000490-37004957 | |
| chr5: 77803754-77808313 | chr9: 77110713-77115927 | |
| chr5: 87966636-87972070 | chr9: 79631327-79640169 | |
| chr5: 87978879-87987810 | chr9: 86150354-86155777 | |
| chr5: 88183225-88187589 | chr9: 91790663-91795611 | |
| chr5: 92921488-92926497 | chr9: 95475297-95479708 | |
| chr5: 92937796-92942216 | chr9: 96106467-96110992 | |
| chr6: 100036542-100041477 | chr9: 96708812-96720186 | |
| chr6: 100895008-100917245 | chr9: 967530-975276 | |
| chr6: 101844767-101849135 | chr9: 97399287-97404067 | |
| chr6: 10379559-10392565 | chr9: 98109365-98114362 | |
| chr6: 106427112-106436459 | chrX: 152610776-152615464 | |
| chr6: 108483672-108499996 | chrX: 67350651-67354923 | |
| chr6: 10879847-10884051 | chrX: 99889300-99893794 | |
| chr6: 110297366-110303267 | | |
| chr6: 117196090-117200705 | | |
| chr6: 117589534-117594279 | | |
| chr6: 117867098-117871530 | | |
| chr6: 127439554-127443760 | | |
| chr6: 134208640-134213218 | | |
| chr6: 134636798-134641021 | | |
| chr6: 137240316-137247442 | | |
| chr6: 1376446-1396170 | | |
| chr6: 137807343-137819223 | | |
| chr6: 138743349-138747593 | | |
| chr6: 150333526-150338278 | | |
| chr6: 150356873-150361394 | | |
| chr6: 154358587-154363008 | | |
| chr6: 168839439-168843699 | | |

1. A method for determining a signal informative for presence or absence of a cancer in a sample obtained from an individual, the method comprising:
obtaining or having obtained sequence reads of cell-free DNA from the sample;
obtaining or having obtained long sequence reads of reference nucleic acids, wherein the long sequence reads of reference nucleic acids are at least 500 bases in length;
attributing long sequence reads of reference nucleic acids to one of two or more different sources of the individual; and
generating phased sequencing information of cell-free DNA by aligning the obtained sequence reads of cell-free DNA to the long sequence reads of reference nucleic acids.

2. The method of claim 1, wherein the phased sequencing information of cell-free DNA comprises methylation sequence information of the cell-free DNA.

3. The method of claim 2, wherein the methylation information of the cell-free DNA comprises methylation statuses for a plurality of genomic sites.

4. The method of claim 2 or 3, wherein the methylation statuses for a plurality of genomic sites comprise coupled genomic sites representing two or more methylated genomic sites originating from a common source.

5. The method of any one of claims 2-4, wherein generating phased sequencing information of cell-free DNA comprises:
comparing methylation statuses of two or more genomic sites from a first source to methylation statuses of the two or more genomic sites from a second source.

6. The method of claim 3 or 4, wherein the plurality of genomic sites comprise a plurality of CpG sites shown in any of Tables 1-4 or portions of the plurality of CpG sites shown in any of Tables 1-4.

7. The method of any one of claims 1-6, wherein the phased sequencing information of cell-free DNA comprises mutation sequence information of the cell-free DNA.

8. The method of claim 7, wherein the mutation sequence information of the cell-free DNA comprises a plurality of mutations present across the plurality of genomic sites.

9. The method of claim 8, wherein the plurality of mutations present across the plurality of genomic sites comprise coupled genomic sites representing two or more mutated genomic sites originating from a common source.

10. The method of claim 8 or 9, wherein the plurality of mutations comprise one or more of a single nucleotide polymorphism (SNP), single nucleotide variant (SNV), insertion, deletion, copy number variation (CNV), duplication, or translocation.

11. The method of any one of claims 1-10, wherein the two or more different sources of the individual comprise a maternal chromosome source or a paternal chromosome source.

12. The method of any one of claims 1-11, wherein the long sequence reads of reference nucleic acids comprise at least 500 bases, at least 1000 bases, at least 2000 bases, at least 3000 bases, at least 4000 bases, at least 5000 bases, at least 6000 bases, at least 7000 bases, at least 8000 bases, at least 9000, at least 10,000 bases, at least 12,000 bases, at least 15,000 bases, at least 20,000 bases, at least 25,000 bases, at least 30,000 bases, at least 40,000 bases, at least 50,000 bases, at least 60,000 bases, at least 70,000 bases, at least 80,000 bases, at least 90,000 bases, or at least 100,000 bases.

13. The method of any one of claims 1-12, wherein the long sequence reads of reference nucleic acids comprise between 5,000 bases and 100,000 bases.

14. The method of any one of claims 1-13, wherein generating phased sequencing information of cell-free DNA does not include aligning the obtained sequence reads of cell-free DNA to a reference genome.

15. The method of any one of claims 1-14, wherein the reference nucleic acids comprise genomic DNA from cells of the individual.

16. The method of claim 15, wherein the cells of the individual comprise peripheral blood mononuclear cells (PBMCs) or polymorphonuclear cells.

17. The method of any one of claims 1-16, wherein the cell-free DNA is obtained from a blood sample, and wherein the reference nucleic acids are obtained from a tissue sample.

18. The method of any one of claims 1-17, wherein obtaining or having obtained sequence reads of cell-free DNA comprises performing an assay, wherein the assay comprises one or more of:
a. sequencing of target nucleic acids via targeted sequencing, whole genome sequencing, or whole genome bisulfite sequencing;
b. a nucleic acid amplification assay; and
c. an assay that generates methylation information.

19. The method of claim 18, wherein the nucleic acid amplification assay is a PCR assay.

20. The method of claim 19, wherein the PCR assay comprises a real-time PCR assay, quantitative real-time PCR (qPCR) assay, digital PCR (dPCR) assay, allele-specific PCR assay, or reverse-transcription PCR assay.

21. The method of any one of claims 1-17, wherein obtaining or having obtained sequence reads of cell-free DNA comprises performing a target enrichment assay.

22. The method of claim 21, wherein the target enrichment assay comprises hybrid capture.

23. The method of any one of claims 18-22, wherein performing the assay comprises:
obtaining bisulfite converted target nucleic acids and/or reference nucleic acids; and
selectively amplifying target regions of the bisulfite converted target nucleic acids and/or reference nucleic acids.

24. The method of any one of claims 1-23, wherein obtaining or having obtained long sequence reads of reference nucleic acids comprises performing nanopore sequencing of reference nucleic acids.

25. The method of any one of claims 1-24, further comprising:
generating the signal informative for presence or absence of a cancer using at least the phased sequencing information of cell-free DNA.

26. The method of any one of claims 1-25, further comprising:
performing longitudinal monitoring of the individual using at least an additional sample obtained from the individual.

27. The method of claim 26, further comprising selecting a therapeutic for administration to the individual based on the longitudinal monitoring.

28. A non-transitory computer readable medium comprising instructions that, when executed by a processor, cause the processor to:
obtain sequence reads of cell-free DNA from the sample;
obtain long sequence reads of reference nucleic acids, wherein the long sequence reads of reference nucleic acids are at least 500 bases in length;
attribute long sequence reads of reference nucleic acids to one of two or more different sources of the individual; and
generate phased sequencing information of cell-free DNA by aligning the obtained sequence reads of cell-free DNA to the long sequence reads of reference nucleic acids.

29. The non-transitory computer readable medium of claim 28, wherein the phased sequencing information of cell-free DNA comprises methylation sequence information of the cell-free DNA.

30. The non-transitory computer readable medium of claim 29, wherein the methylation information of the cell-free DNA comprises methylation statuses for a plurality of genomic sites.

31. The non-transitory computer readable medium of claim 28 or 29, wherein the methylation statuses for a plurality of genomic sites comprise coupled genomic sites representing two or more methylated genomic sites originating from a common source.

32. The non-transitory computer readable medium of any one of claims 29-31, wherein generating phased sequencing information of cell-free DNA comprises:
comparing methylation statuses of two or more genomic sites from a first source to methylation statuses of the two or more genomic sites from a second source.

33. The non-transitory computer readable medium of claim 30 or 31, wherein the plurality of genomic sites comprise a plurality of CpG sites shown in any of Tables 1-4 or portions of the plurality of CpG sites shown in any of Tables 1-4.

34. The non-transitory computer readable medium of any one of claims 28-33, wherein the phased sequencing information of cell-free DNA comprises mutation sequence information of the cell-free DNA.

35. The non-transitory computer readable medium of claim 34, wherein the mutation sequence information of the cell-free DNA comprises a plurality of mutations present across the plurality of genomic sites.

36. The non-transitory computer readable medium of claim 35, wherein the plurality of mutations present across the plurality of genomic sites comprise coupled genomic sites representing two or more mutated genomic sites originating from a common source.

37. The non-transitory computer readable medium of claim 35 or 36, wherein the plurality of mutations comprise one or more of a single nucleotide polymorphism (SNP), single nucleotide variant (SNV), insertion, deletion, copy number variation (CNV), duplication, or translocation.

38. The non-transitory computer readable medium of any one of claims 28-37, wherein the two or more different sources of the individual comprise a maternal chromosome source or a paternal chromosome source.

39. The non-transitory computer readable medium of any one of claims 28-38, wherein the long sequence reads of reference nucleic acids comprise at least 500 bases, at least 1000 bases, at least 2000 bases, at least 3000 bases, at least 4000 bases, at least 5000 bases, at least 6000 bases, at least 7000 bases, at least 8000 bases, at least 9000, at least 10,000 bases, at least 12,000 bases, at least 15,000 bases, at least 20,000 bases, at least 25,000 bases, or at least 30,000 bases.

40. The non-transitory computer readable medium of any one of claims 28-39, wherein the long sequence reads of reference nucleic acids comprise between 5,000 bases and 100,000 bases.

41. The non-transitory computer readable medium of any one of claims 28-40, wherein the instructions that cause the processor to generate phased sequencing information of cell-free DNA does not include instructions that cause the processor to align the obtained sequence reads of cell-free DNA to a reference genome.

42. The non-transitory computer readable medium of any one of claims 28-41, wherein the reference nucleic acids comprise genomic DNA from cells of the individual.

43. The non-transitory computer readable medium of claim 42, wherein the cells of the individual comprise peripheral blood mononuclear cells (PBMCs) or polymorphonuclear cells.

44. The non-transitory computer readable medium of any one of claims 28-43, wherein the cell-free DNA is obtained from a blood sample, and wherein the reference nucleic acids are obtained from a tissue sample.

45. A system comprising:
a processor;
a data storage comprising sequence reads of cell-free DNA from a sample obtained from an individual and long sequence reads of reference nucleic acids, wherein the long sequence reads of reference nucleic acids are at least 500 bases in length; and
a non-transitory computer readable medium comprising instructions that, when executed by the processor, cause the processor to:
attribute long sequence reads of reference nucleic acids to one of two or more different sources of the individual; and
generate phased sequencing information of cell-free DNA by aligning the obtained sequence reads of cell-free DNA to the long sequence reads of reference nucleic acids

46. The system of claim 45, wherein the phased sequencing information of cell-free DNA comprises methylation sequence information of the cell-free DNA.

47. The system of claim 46, wherein the methylation information of the cell-free DNA comprises methylation statuses of a plurality of genomic sites.

48. The system of claim 46 or 47, wherein the methylation statuses for a plurality of genomic sites comprise coupled genomic sites representing two or more methylated genomic sites originating from a common source.

49. The system of any one of claims 45-48, wherein generating phased sequencing information of cell-free DNA comprises:
comparing methylation statuses of two or more genomic sites from a first source to methylation statuses of the two or more genomic sites from a second source.

50. The system of claim 47 or 48, wherein the plurality of genomic sites comprise a plurality of CpG sites shown in any of Tables 1-4 or portions of the plurality of CpG sites shown in any of Tables 1-4.

51. The system of any one of claims 45-50, wherein the phased sequencing information of cell-free DNA comprises mutation sequence information of the cell-free DNA.

52. The system of claim 51, wherein the mutation sequence information of the cell-free DNA comprises a plurality of mutations present across the plurality of genomic sites.

53. The system of claim 52, wherein the plurality of mutations present across the plurality of genomic sites comprise coupled genomic sites representing two or more mutated genomic sites originating from a common source.

54. The system of claim 52 or 53, wherein the plurality of mutations comprise one or more of a single nucleotide polymorphism (SNP), single nucleotide variant (SNV), insertion, deletion, copy number variation (CNV), duplication, or translocation.

55. The system of any one of claims 45-54, wherein the two or more different sources of the individual comprise a maternal chromosome source or a paternal chromosome source.

56. The system of any one of claims 45-55, wherein the long sequence reads of reference nucleic acids comprise at least 500 bases, at least 1000 bases, at least 2000 bases, at least 3000 bases, at least 4000 bases, at least 5000 bases, at least 6000 bases, at least 7000 bases, at least 8000 bases, at least 9000, at least 10,000 bases, at least 12,000 bases, at least 15,000 bases, at least 20,000 bases, at least 25,000 bases, or at least 30,000 bases.

57. The system of any one of claims 45-56, wherein the long sequence reads of reference nucleic acids comprise between 5,000 bases and 100,000 bases.

58. The system of any one of claims 45-57, wherein generating phased sequencing information of cell-free DNA does not include aligning the obtained sequence reads of cell-free DNA to a reference genome.

59. The system of any one of claims 45-58, wherein the reference nucleic acids comprise genomic DNA from cells of the individual.

60. The system of claim 59, wherein the cells of the individual comprise peripheral blood mononuclear cells (PBMCs) or polymorphonuclear cells.

61. The system of any one of claims 45-60, wherein the cell-free DNA is obtained from a blood sample, and wherein the reference nucleic acids are obtained from a tissue sample.
